(19) United States
(12) Patent Application Publication
(10) Pub. No.: US 2021/0364523 A1
(43) Pub. Date: Nov. 25, 2021

(54) GENETICALLY ENCODED RATIOMETRIC FLUORESCENT BARCODES

(71) Applicant: THE TRUSTEES OF COLUMBIA UNIVERSITY IN THE CITY OF NEW YORK, New York, NY (US)

(72) Inventors: Andrew V. Anzalone, New York, NY (US); Virginia W. Cornish, New York, NY (US); Miguel Jimenez, Cambridge, MA (US)

(73) Assignee: THE TRUSTEES OF COLUMBIA UNIVERSITY IN THE CITY OF NEW YORK, New York, NY (US)

(21) Appl. No.: 17/347,285

(22) Filed: Jun. 14, 2021

Related U.S. Application Data
(63) Continuation of application No. PCT/US2019/066634, filed on Dec. 16, 2019.
(60) Provisional application No. 62/779,993, filed on Dec. 14, 2018.

(51) Int. Cl. G01N 33/58; C12N 15/62; G01N 21/64

(52) U.S. Cl. CPC G01N 33/582; G01N 2021/6439; G01N 21/6428; C12N 15/62

(57) ABSTRACT

The disclosed subject matter relates to nucleic acid constructs that encode one or more fluorescent proteins, which can function as intracellular fluorescent tags, specifically for flow cytometry and fluorescence microscopy. The present disclosure further provides genetically-engineered cells that include one or more nucleic acid constructs and methods for using such genetically-engineered cells and nucleic acid constructs.

### CROSS-REFERENCE TO RELATED APPLICATIONS

This application is a continuation of International Patent Application No. PCT/US2019/066634, filed Dec. 16, 2019, which claims priority to U.S. Provisional Application No. 62/779,993, filed on Dec. 14, 2018, the contents of each of which are hereby incorporated by reference in their entireties, and to each which priority is claimed.

### GRANT FUNDING

This invention was made with government support under AI110794 and CA174357 awarded by the National Institutes of Health and 1144155 awarded by the National Science Foundation. The government has certain rights in the invention.

### SEQUENCE LISTING

The instant application contains a Sequence Listing which has been submitted electronically in ASCII format and is hereby incorporated by reference in its entirety. Said ASCII copy, created on Jun. 14, 2021, is named 070050_6507_SL.txt and is 47,040 bytes in size. The Sequence Listing does not extend beyond the scope of the specification and thus does not contain new matter.

### TECHNICAL FIELD

The disclosed subject matter relates to intracellular fluorescent tags, specifically for flow cytometry and fluorescence microscopy.

### BACKGROUND

Networks of dynamically interacting cells and species govern many biological processes with relevance to human health and ecology. Understanding these cellular networks, such as the microbiome and the immune system, can enable the elucidation and prediction of complex behaviors like microbial drug metabolism, pathogen engraftment, inflammatory disease and cancer. One component to understanding these network processes is tracking the identities and phenotypes of cells over time. While certain cell barcoding methods, such as those based on sequencing or exogenous labeling, can capture system-wide snapshots of cell populations, these techniques are not necessarily easily adapted for measuring time-resolved rates of change in cell phenotype or population composition.

These cell barcoding tools should be highly scalable, resolvable, self-renewing, and conveniently analyzed in a non-destructive manner using widely accessible instruments. Certain fluorescent proteins (FPs) can be components for building cellular barcodes because they meet several of these criteria. First, they are genetically encoded and therefore do not dilute over multiple cell generations. Second, they emit fluorescence signals that can be easily measured directly form the sample with microscopy and flow cytometry. Finally, a wide variety of FPs are available with different colors and biophysical properties. However, despite their exceptional qualities, FPs can have broad fluorescence spectra that limit the number of variants that can be resolved simultaneously. This can restrict FP-based barcoding to experiments containing only a small number (3 to 5) of cell types. While elegant combinatorial approaches can address this challenge, certain methods exhaust most available fluorescence channels because of their dependence on three or more FP colors. This can limit their use alongside other fluorescent reporters and places an upper bound on their scalability. Moreover, certain methods, such as those based on Brainbow, can face scaling challenges since discrimination between similar hues becomes increasingly difficult with increasing barcode number, requiring the use of additional spatial information to fully identify specific cells. Finally, to generate more than three colors, these methods can rely on stochastic genetic integration of multiple expression units, which makes barcode generation unpredictable and unassignable a priori. The above limitations can place restrictions on how and where FP markers can be used for multiplexed cellular tracking.

As efforts progress in studying, modeling, and even building multicellular networks, a next generation of multiplex cell barcoding tools is needed for time-resolved cellular identification, lineage tracing and phenotypic reporting from intact biological systems.

### SUMMARY

The present disclosure provides a nucleic acid construct for labeling one or more cells, e.g., within a population of cells. In certain embodiments, the nucleic acid construct includes a first nucleic acid segment encoding a first fluorescent protein, a second nucleic acid segment encoding a second fluorescent protein, a third nucleic acid segment including a slippery site, e.g., positioned upstream of the second nucleic acid segment encoding the second fluorescent protein, and a fourth nucleic acid segment including a frameshift stimulatory sequence, e.g., positioned upstream of the second nucleic acid segment encoding the second fluorescent protein. In certain embodiments, the nucleic acid construct includes a fifth nucleic acid segment encoding a stop codon, e.g., positioned upstream of the second nucleic acid segment encoding the second fluorescent protein. In certain embodiments, the fifth nucleic acid segment, e.g., the stop codon, is in frame with the second nucleic acid segment encoding the second fluorescent protein.

In certain embodiments, a nucleic acid construct of the present disclosure can include one or more nucleic acid segments encoding a third fluorescent protein, a fourth fluorescent protein, a fifth fluorescent protein, a sixth fluorescent protein, a seventh fluorescent protein, an eighth fluorescent protein, a ninth fluorescent protein and/or a tenth fluorescent protein. In certain embodiments, the nucleic acid construct can include one or more nucleic acid segments encoding a second slippery site, a third slippery site, a fourth slippery site, a fifth slippery site, a sixth slippery site, a seventh slippery site, an eighth slippery site and/or a ninth slippery site. In certain embodiments, the nucleic acid construct can include one or more nucleic acid segments encoding a second stop codon, a third stop codon, a fourth stop codon, a fifth stop codon, a sixth stop codon, a seventh stop codon, an eighth stop codon and/or a ninth stop codon. In certain embodiments, the nucleic acid construct can include a second frameshift stimulatory sequence, a third frameshift stimulatory sequence, a fourth frameshift stimulatory sequence, a fifth frameshift stimulatory sequence, a sixth frameshift stimulatory sequence, a seventh frameshift stimulatory sequence, an eighth frameshift stimulatory sequence and/or a ninth frameshift stimulatory sequence. For example, but not by way of limitation, a nucleic acid construct of the present disclosure can further include a sixth nucleic acid segment encoding a third fluorescent protein, a seventh nucleic acid segment encoding a second slippery site, an eighth nucleic acid segment encoding a second frameshift stimulatory sequence and a ninth nucleic acid segment encoding a second stop codon.

In certain embodiments, the first fluorescent protein, the second fluorescent protein and/or the third fluorescent protein can be a fluorescent protein selected from the group consisting of a green fluorescent protein (GFP), a red fluorescent protein (RFP), a blue fluorescent protein (BFP), a cyan fluorescent protein (CFP), a yellow fluorescent protein (YFP), an orange fluorescent protein (OFP), a far-red fluorescent protein, a near-infrared fluorescent protein, a long stokes shift fluorescent protein, a photo-activatable fluorescent protein, a photoconvertible fluorescent protein, a photoswitchable fluorescent protein, a timer fluorescent protein and mutants or variants thereof. In certain embodiments, the first fluorescent protein, the second fluorescent protein and/or third fluorescent protein are different fluorescent proteins, e.g., the first fluorescent protein, the second fluorescent protein and/or third fluorescent protein are dependently selected from a GFP, an RFP, a BFP, a CFP, a YFP, an OFP, a far-red fluorescent protein, a near-infrared fluorescent protein, a long stokes shift fluorescent protein, a photo-activatable fluorescent protein, a photoconvertible fluorescent protein, a photoswitchable fluorescent protein, a timer fluorescent protein and mutants or variants thereof. In certain embodiments, first fluorescent protein, second fluorescent protein and/or third fluorescent protein are dependently selected from GFP, sfGFP, deGFP, eGFP, Venus, mVenus, YFP, Cerulean, Citrine, CFP, eYFP, eCFP, RFP, mRFP, mCherry, mmCherry, mTurquoise2, mKO2, BFP, mTagBFP2 and mutants or variants thereof. In certain embodiments, the first fluorescent protein is a GFP, e.g., eGFP, and the second fluorescent protein is an RFP, e.g., mCherry. In certain embodiments, the first fluorescent protein is a GFP, e.g., eGFP, the second fluorescent protein is an RFP, e.g., mCherry, and the third fluorescent protein is a BFP, e.g., mTagBFP2.

The present disclosure further provides genetically-engineered cells that include one or more or two or more nucleic acid constructs disclosed herein. In certain embodiments, the cell is a prokaryotic cell or a eukaryotic cell. In certain embodiments, the cell is selected from the group consisting of a mammalian cell, a plant cell and a fungal cell. In certain embodiments, the cell is a mammalian cell. In certain embodiments, the cell is a plant cell. In certain embodiments, the cell is a fungal cell. In certain embodiments, the fungal cell is a species of the phylum Ascomycota, e.g., Saccharomyces cerevisiae, Saccharomyces castellii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipites, Komagataella (Pichia) pastoris, Candida (Pichia) guilliermondii, Candida parapsilosis, Candida auris, Yarrowia hpolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinereal, Beauvaria bassiana, Neurospora crassa, Sporothrix scheckii, Magnaporthe oryzea, Dactylellina haptotyla, Fusarium graminearum, Capronia coronate and combinations thereof. In certain embodiments, the cell is Saccharomyces cerevisiae. In certain embodiments, the first fluorescent protein and the second fluorescent protein are expressed in the genetically-engineered cell at a ratio of about 1:1,000 to about 1,000:1, e.g., about 1:100 to about 100:1. In certain embodiments, the first fluorescent protein and the third fluorescent protein are expressed in the genetically-engineered cell at a ratio of about 1:1,000 to about 1,000:1, e.g., about 1:100 to about 100:1. In certain embodiments, the second fluorescent protein and the third fluorescent protein are expressed in the genetically-engineered cell at a ratio of about 1:1,000 to about 1,000:1, e.g., about 1:100 to about 100:1.

The present disclosure further provides a cell population that includes one or more genetically-engineered cells described herein. The present disclosure further provides kits that include one or more nucleic acid constructs or genetically-engineered cells described herein.

The present disclosure further provides methods for labeling a cell that includes introducing one or more nucleic acid constructs described herein into the cell. In certain embodiments, the method can include expressing two or more fluorescent proteins from the one or more nucleic acid constructs. In certain embodiments, the method can further include determining the ratio of fluorescent between the two or more fluorescent proteins expressed in the cell.

### BRIEF DESCRIPTION OF THE DRAWINGS

FIG. 1A-1C. FIG. 1A is schematic of a DNA construct controlling the ratios between a first fluorescent protein (FP-1) and a second fluorescent protein (FP-2) (FRAME-tag). FIG. 1B is a schematic of the translation of mRNA from the DNA construct and the resulting protein expression. FIG. 1C is a graph depicting frameshift efficiency of different constructs evaluated by flow cytometry.

FIG. 2A-2B. FIG. 2A is a graph of the fluorescence of a population of cells, the population consisting of different transformed subpopulations transformed with different FRAME-tags. FIG. 2B is a graph of the fluorescence of a population of cells consisting of different transformed subpopulations transformed with three-fluorescent-protein FRAME-tags. The data were measured using flow cytometry.

FIG. 3A-3C. FIG. 3A is a series of fluorescence microscopy images of a transformed yeast cell population transformed with different FRAME-tags. FIG. 3B is a graph of the fluorescence of the entire cell population discussed in A measured using flow cytometry. FIG. 3C is a false color version of the merged FIG. 3A, where the cells are colored based on the FRAME-tag as identified based on the ratio of the fluorescence of the first fluorescent protein to the fluorescence of the second fluorescent protein.

FIG. 4A-4D. FIG. 4A is a schematic of a series of different individual yeast strains expressing varying levels of the protein Gal4, where each yeast strain was transformed with a different FRAME-tag. FIG. 4B is a graph showing the change in in yeast strain populations shown in FIG. 4A tracked by flow cytometry in response to different culture conditions. FIG. 4C is a graph showing changes in population distribution (PD) index based on changing culture conditions. FIG. 4D is a graph of the change in population fraction of the different yeast strains described in FIG. 4A over time in different culture conditions. The data were measured using flow cytometry.

FIG. 5A-5C. FIG. 5A is a schematic showing a series of yeast transformed with different promoters. Each promotor was linked to a different, known, FRAME-tag. FIG. 5B is a pair of graphs, the left graph showing the population of transformed cells as measured by fluorescence of a first FRAME-tag fluorescent protein as a function of a second FRAME-tag fluorescent protein. The right graph shows the promotor activity. FIG. 5C shows the change in promotor activity as a function of different culture conditions and different yeast strains, the yeast strains described in FIG. 5A.

FIG. 6 shows a graph showing the existing set of FRAME-tags and the theoretical fluorescence ratios that could be used to expand the number of resolvable FRAME-tags.

FIG. 7A-7B. FIG. 7A is a schematic of the frameshift module architecture. FIG. 7B is a schematic showing the predicted structure of different frameshift stimulatory signals.

FIG. 8A-8D. FIG. 8A is a schematic showing the frameshift module position. FIG. 8B is a graph showing the level of fluorescence of the second fluorescent protein located after different frameshift modules. FIG. 8C is a chart showing the frameshift efficiency of different frameshift modules. FIG. 8D is a graph showing the level of overlap between the fluorescent protein fluorescence ratios each frameshift module produces.

FIG. 9A-9B. FIG. 9A is a graph depicting the fluorescence of cells transformed with a FRAME-tag expressed from a plasmid. FIG. 9B is a graph depicting the fluorescence of cells transformed with a FRAME-tag expressed from the chromosome. Fluorescence was normalized by side scatter; contours represent 5% quantiles.

FIG. 10A-10B. FIG. 10A is a graph depicting the intensity of fluorescence of the first protein in a FRAME-tag as a function of frameshift efficiency. FIG. 10B is a graph depicting the intensity of fluorescence as a function of the position of the frameshift module.

FIG. 11 shows a graph depicting fluorescence data collected from a population of yeast with 23 subpopulations, 22 of the subpopulations transformed with a different FRAME-tag.

FIG. 12A-12B. FIG. 12A is a graph depicting the fluorescence of the first protein in a FRAME-tag and the fluorescence of the second protein in a FRAME-tag as a function of the expression of three different fluorescent proteins: mBFP2, mTurquoise2, mVenus, and mKO2. FIG. 12B is a histogram depicting the BF2 plot of FIG. 12A.

FIG. 13A-13B. FIG. 13A is a graph depicting the fluorescence of yeast strains transformed with different three-fluorescent protein FRAME-tags. FIG. 13B is a graph showing the three possible two-dimensional projections of the three-dimensional figure of FIG. 13A.

FIG. 14A-14G. FIG. 14A is a flowchart showing a method of distinguishing different cell populations based on their fluorescence. FIG. 14B is a set of graphs showing where the fluorescent data sets Q1Q2R and Q1Q4R were bisected to yield data set Q1. FIG. 14C is a set of graphs showing where the fluorescent data sets Q1 and Q1-FT4 were bisected to yield data set Q1-FT4-FT16. FIG. 14D is a set of graphs showing where the fluorescent data set Q1-FT4-FT16 and q1q2 were bisected to yield data set q1. FIG. 14E is a set of graphs showing where the fluorescent data sets q1 and FT.2.3.8 were bisected to yield data set FT.2.8. FIG. 14F is a set of graphs showing where the fluorescent data set FT.2.8 was bisected to yield data set FT2p. FIG. 14G is a graph depicting the identified cell population FT2, which was identified because there is no 3^{rd} lowest local minimum.

FIG. 15A-15D. FIG. 15A is a graph depicting the sensitivity of the method depicted by the flowchart of FIG. 14. FIG. 15B is a graph depicting the positive predictive value (PPV) of the method depicted by the flowchart of FIG. 14. FIG. 15C is a graph depicting the specificity of the method depicted by the flowchart of FIG. 14. FIG. 15D is a graph depicting the negative predictive value (NPV) of the method depicted by the flowchart of FIG. 14.

FIG. 16A-16B. FIG. 16A is a graph depicting the PPV of the method depicted by the flowchart of FIG. 14 as a function of the population fraction of the identified cell population. FIG. 16B is a graph depicting the data of FIG. 16A at a population fraction of 0.0008 for 21 target strains transformed with different FRAME-tags.

FIG. 17A-17C. FIG. 17A is a set of microscopy images taken either of yEGFP fluorescence, mCherry fluorescence, merged, brightfield, or false-color. False colors correspond to the different FRAME-tag transformed in the yeast cell as identified by fluorescence ratio. FIG. 17B is a widefield image of the false-color image of FIG. 17A. FIG. 17C is a graph depicting grouping analysis based on widefield fluorescent spectrometry images of yeast transformed with different FRAME-tags.

FIG. 18A-18C. FIG. 18A is a set of microscopy images taken either of yEGFP fluorescence, mCherry fluorescence, merged, brightfield, or false-color. False colors correspond to the different FRAME-tag transformed yeast cells as identified by fluorescence ratio. FIG. 18B is a widefield image of the false-color image of FIG. 18A. FIG. 18C is a graph depicting grouping analysis based on widefield fluorescent spectrometry images of yeast transformed with different FRAME-tags.

FIG. 19 shows a schematic depicting a method of tagging cell phenotypes in new cell strains.

FIG. 20A-20C. FIG. 20A is a schematic depicting a construct introduced to a MaV203 yeast strain. FIG. 20B is a graph depicting the survival of the different yeast strains in different culture conditions. FIG. 20C is a graph depicting the survival of the different yeast strains under competitive conditions.

FIG. 21A-21D. FIG. 21A-C is a graph depicting the Population Distribution Index (PDI,A), OD600 (B), and both PDI and OD600 (C) of different yeast strains as a function of histidine (His), 5-fluoroorotic acid (5-FOA), and 3-aminotriazole (3-AT). FIG. 21D is a schematic depicting the numbering of each condition.

FIG. 22A-22U provides histograms profiling responses of mixtures of promoter-mTagBFP2 reporter strains co-exposed to standard conditions (grey), (A) media with 50 mM dithiothreitol (DTT, red), (B) heat shock at 42° C. (red), (C) media with 400 μM cobalt chloride (CoCl₂, red), (D) 500 μM copper sulfate (CuSO₄, red), (E) an alternate carbon source (2% galactose, red), (F) a mixed carbon source (2% galactose/2% raffinose, red), (G) an increased amount of the carbon source (10% glucose, red), (H) a non-standard carbon source (2% ethanol, red) for 6 hours, (I) a non-standard carbon source (2% glucose, red), (J) standard media containing 5% dimethylslfoxide (DMSO, red), (K) media with 5 μM FK506 (red), (L) media lacking an essential amino acid (-histidine, red), (M) osmotic shock with in media containing 0.7 M sodium chloride (NaCl, red), (N) media with 5 units zymolyase a cell wall-degrading enzyme (red), (O) oxidative shock with in media containing 1 mM hydrogen peroxide (H₂O₂, red), (P) media with 5 μM α-factor a yeast peptide pheromone (red), (Q) media containing 0.1% methyl methanesulfonate a genotoxin (MMS, red), (R) media with 0.01% 5-fluoroorotic acid which is converted to the antimetabolite 5-fluorouracil (5-FOA, red), (S) media containing 50 mM 3-amino-1,2,4-triazole a competitive inhibitor of histidine metabolism (3-AT, red), (T) media with 40 mM theophylline (red), or (U) media containing 50% human urine (red), all for 6 hours. The mTagBFP2 fluorescent signal was normalized by side scatter and plotted as arbitrary logical units.

### DETAILED DESCRIPTION

The present disclosure provides for nucleic acid constructs encoding two or more proteins, e.g., fluorescent proteins, that when introduced into a cell, e.g., yeast cell, results in the genetically-engineered cell expressing a ratio of the two proteins, e.g., fluorescent proteins.

For purposes of clarity of disclosure and not by way of limitation, the detailed description is divided into the following subsections:

I. Definitions;

II. Nucleic Acid Constructs;

III. Cells;

IV. Methods of Use;

V. Kits.

### I. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.”

The terms “comprise(s),” “include(s),” “having,” “has,” “can,” “contain(s),” and variants thereof, as used herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “expression” or “expresses,” as used herein, refer to transcription and translation occurring within a cell, e.g., yeast cell. The level of expression of a gene and/or nucleic acid in a cell can be determined on the basis of either the amount of corresponding mRNA that is present in the cell or the amount of the protein, e.g., fluorescent protein, encoded by the gene and/or nucleic acid that is produced by the cell. For example, mRNA transcribed from a gene and/or nucleic acid is desirably quantitated by northern hybridization. Sambrook et al., Molecular Cloning: A Laboratory Manual, pp. 7.3-7.57 (Cold Spring Harbor Laboratory Press, 1989). Protein encoded by a gene and/or nucleic acid can be quantitated either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989).

As used herein, “polypeptide” refers generally to peptides and proteins having about three or more amino acids. The polypeptides can be endogenous to the cell, or preferably, can be exogenous, meaning that they are heterologous, i.e., foreign, to the cell being utilized, such as a synthetic peptide and/or protein, e.g., a fluorescent protein. In certain embodiments, synthetic peptides are used, more preferably those which are directly secreted into the medium.

The term “protein” is meant to refer to a sequence of amino acids for which the chain length is sufficient to produce the higher levels of tertiary and/or quaternary structure. This is to distinguish from “peptides” that typically do not have such structure. Typically, the protein herein will have a molecular weight of at least about 15-100 kD, e.g., closer to about 15 kD. In certain embodiments, a protein can include at least about 50, about 60, about 70, about 80, about 90, about 100, about 200, about 300, about 400 or about 500 amino acids. Examples of proteins encompassed within the definition herein include all proteins, and, in general proteins that contain one or more disulfide bonds, including multi-chain polypeptides including one or more inter- and/or intrachain disulfide bonds. In certain embodiments, proteins can include other post-translation modifications including, but not limited to, glycosylation and lipidation. See, e.g., Prabakaran et al., WIREs Syst Biol Med (2012), which is incorporated herein by reference in its entirety.

As used herein the term “amino acid,” “amino acid monomer” or “amino acid residue” refers to organic compounds composed of amine and carboxylic acid functional groups, along with a side-chain specific to each amino acid. In particular, alpha- or α-amino acid refers to organic compounds in which the amine (—NH2) is separated from the carboxylic acid (—COOH) by a methylene group (—CH2), and a side-chain specific to each amino acid connected to this methylene group (—CH2) which is alpha to the carboxylic acid (—COOH). Different amino acids have different side chains and have distinctive characteristics, such as charge, polarity, aromaticity, reduction potential, hydrophobicity, and pKa. Amino acids can be covalently linked to form a polymer through peptide bonds by reactions between the carboxylic acid group of the first amino acid and the amine group of the second amino acid. Amino acid in the sense of the disclosure refers to any of the twenty plus naturally occurring amino acids, non-natural amino acids, and includes both D and L optical isomers.

The terms “nucleic acid,” “nucleic acid molecule,” “nucleic acid construct” or “polynucleotide,” used interchange herein, include any compound and/or substance that includes a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including, e.g., complementary DNA (cDNA) and genomic DNA, ribonucleic acid (RNA), in particular messenger RNA (mRNA), synthetic forms of DNA or RNA, and mixed polymers including two or more of these molecules. The nucleic acid molecule can be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of fluorescent proteins of the disclosure in vitro and/or in vivo, e.g., in a yeast cell. Such DNA (e.g., cDNA) or RNA (e.g., mRNA) vectors, can be unmodified or modified. For example, mRNA can be chemically modified to enhance the stability of the RNA vector and/or expression of the encoded molecule.

As used herein, the term “vector” refers to a nucleic acid molecule capable of transporting another nucleic acid to which it has been linked.

As used herein, the term “recombinant cell” refers to cells which have some genetic modification from the original parent cells from which they are derived. Such cells can also be referred to as “genetically-engineered cells.” Such genetic modification can be the result of an introduction of a heterologous nucleic acid for expression of a fluorescent protein, e.g., two or more fluorescent proteins.

As used herein, the term “recombinant protein” refers generally to peptides and proteins. Such recombinant proteins are “heterologous,” i.e., foreign to the cell being utilized, such as a heterologous secretory peptide produced by a yeast cell.

As used herein, “sequence identity” or “identity” in the context of two polynucleotide or polypeptide sequences makes reference to the nucleotide bases or amino acid residues in the two sequences that are the same when aligned for maximum correspondence over a specified comparison window. When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule.

As used herein, “percentage of sequence identity” means the value determined by comparing two optimally aligned sequences over a comparison window, wherein the portion of the polynucleotide sequence in the comparison window can include additions or deletions (gaps) as compared to the reference sequence (which does not include additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

As understood by those skilled in the art, determination of percent identity between any two sequences can be accomplished using certain well-known mathematical algorithms. Non-limiting examples of such mathematical algorithms are the algorithm of Myers and Miller, the local homology algorithm of Smith et al.; the homology alignment algorithm of Needleman and Wunsch; the search-for-similarity-method of Pearson and Lipman; the algorithm of Karlin and Altschul, modified as in Karlin and Altschul. Computer implementations of suitable mathematical algorithms can be utilized for comparison of sequences to determine sequence identity. Such implementations include, but are not limited to: CLUSTAL, ALIGN, GAP, BESTFIT, BLAST, FASTA, among others identifiable by skilled persons.

As used herein, “reference sequence” is a defined sequence used as a basis for sequence comparison. A reference sequence can be a subset or the entirety of a specified sequence; for example, as a segment of a full-length protein or protein fragment. A reference sequence can be, for example, a sequence identifiable in a database such as GenBank and UniProt and others identifiable to those skilled in the art.

The term “operative connection” or “operatively linked,” as used herein, with regard to regulatory sequences of a gene indicate an arrangement of elements in a combination enabling production of an appropriate effect. With respect to genes and regulatory sequences, an operative connection indicates a configuration of the genes with respect to the regulatory sequence allowing the regulatory sequences to directly or indirectly increase or decrease transcription or translation of the genes. In particular, in certain embodiments, regulatory sequences directly increasing transcription of the operatively linked gene, include promoters typically located on a same strand and upstream on a DNA sequence (towards the 5′ region of the sense strand), adjacent to the transcription start site of the genes whose transcription they initiate. In certain embodiments, regulatory sequences directly increasing transcription of the operatively linked gene or gene cluster include enhancers that can be located more distally from the transcription start site compared to promoters, and either upstream or downstream from the regulated genes, as understood by those skilled in the art. Enhancers are typically short (50-1500 bp) regions of DNA that can be bound by transcriptional activators to increase transcription of a particular gene. Typically, enhancers can be located up to 1 Mbp away from the gene, upstream or downstream from the start site.

As would be understood by those skilled in the art, the term “codon optimization,” as used herein, refers to the introduction of synonymous mutations into codons of a protein-coding gene in order to improve protein expression in expression systems of a particular organism, such as a cell of a species of the phylum Ascomycota, in accordance with the codon usage bias of that organism. The term “codon usage bias” refers to differences in the frequency of occurrence of synonymous codons in coding DNA. The genetic codes of different organisms are often biased towards using one of the several codons that encode a same amino acid over others—thus using the one codon with, a greater frequency than expected by chance. Optimized codons in microorganisms, such as Saccharomyces cerevisiae, reflect the composition of their respective genomic tRNA pool. The use of optimized codons can help to achieve faster translation rates and high accuracy.

In the field of bioinformatics and computational biology, many statistical methods have been discussed and used to analyze codon usage bias. Methods such as the ‘frequency of optimal codons’ (Fop), the Relative Codon Adaptation (RCA) or the ‘Codon Adaptation Index’ (CAI) are used to predict gene expression levels, while methods such as the ‘effective number of codons’ (Nc) and Shannon entropy from information theory are used to measure codon usage evenness. Multivariate statistical methods, such as correspondence analysis and principal component analysis, are widely used to analyze variations in codon usage among genes. There are many computer programs to implement the statistical analyses enumerated above, including CodonW, GCUA, INCA, and others identifiable by those skilled in the art. Several software packages are available online for codon optimization of gene sequences, including those offered by companies such as GenScript, EnCor Biotechnology, Integrated DNA Technologies, ThermoFisher Scientific, among others known those skilled in the art.

The terms “detect” or “detection,” as used herein, indicates the determination of the existence and/or presence of a target, e.g., fluorescent protein, in a limited portion of space, including but not limited to a sample, a reaction mixture, a molecular complex and a substrate. The “detect” or “detection” as used herein can include determination of a property, e.g., chemical and/or biological property, of the target, including but not limited to ability to interact, and in particular bind, other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified.

As used herein, the term “a population of cells” or “a cell population” refers to a group of at least two cells. In certain non-limiting examples, a cell population can include at least about 10, at least about 100, at least about 200, at least about 300, at least about 400, at least about 500, at least about 600, at least about 700, at least about 800, at least about 900, at least about 1000 cells, at least about 5,000 cells or at least about 10,000 cells or at least about 100,000 cells or at least about 1,000,000 cells. The population can be a pure population including one cell type. Alternatively, the population can include more than one cell type, for example a mixed cell population.

As used herein, the term “in vitro” refers to an artificial environment and to processes or reactions that occur within an artificial environment. In vitro environments exemplified, but are not limited to, test tubes and cell cultures.

As used herein, the term “derived from” or “established from” or “differentiated from” when made in reference to any cell disclosed herein refers to a cell that was obtained from (e.g., isolated, purified, etc.) a parent cell in a cell line, tissue (such as a dissociated embryo, or fluids using any manipulation, such as, without limitation, single cell isolation, cultured in vitro, treatment and/or mutagenesis using for example proteins, chemicals, radiation, infection with virus, transfection with DNA sequences, such as with a morphogen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure and the like.

### II. Nucleic Acid Constructs

The present disclosure relates to a nucleic acid construct, e.g., DNA construct, that can be introduced into one or more cells to express a fluorescent protein. In certain embodiments, the nucleic acid construct encodes for at least two different fluorescent proteins. For example, but not by way of limitation, the nucleic acid construct encodes for at least three different fluorescent proteins, at least four different fluorescent proteins, at least five different fluorescent proteins, at least six different fluorescent proteins, at least seven different fluorescent proteins, at least eight different fluorescent proteins, at least nine different fluorescent proteins or at least ten different fluorescent proteins. In certain embodiments, a nucleic acid construct of the present disclosure encodes about two or more fluorescent proteins, e.g., about two or more, about three or more, about four or more, about five or more, about six or more, about seven or more, about eight or more, about nine or more or about ten or more fluorescent proteins.

In certain embodiments, a nucleic acid construct of the present disclosure encodes from about two to about ten different fluorescent proteins. For example, but not by way of limitation, a nucleic acid construct of the present disclosure encodes from about two to about nine different fluorescent proteins, about two to about eight different fluorescent proteins, about two to about seven different fluorescent proteins, about two to about six different fluorescent proteins, about two to about five different fluorescent proteins, about two to about four different fluorescent proteins or about two to about three different fluorescent proteins.

In certain embodiments, a nucleic acid construct encodes for two different fluorescent proteins, e.g., the nucleic acid construct includes a first nucleic acid segment encoding a first fluorescent protein and a second nucleic acid segment encoding a second fluorescent protein. In certain embodiments, a nucleic acid construct encodes for three different fluorescent proteins, e.g., the nucleic acid construct further includes a third nucleic acid segment encoding a third fluorescent protein. In certain embodiments, a nucleic acid construct encodes for four different fluorescent proteins, e.g., the nucleic acid construct further includes a fourth nucleic acid segment encoding a fourth fluorescent protein. In certain embodiments, a nucleic acid construct encodes for five different fluorescent proteins, e.g., the nucleic acid construct further includes a fifth nucleic acid segment encoding a fifth fluorescent protein. In certain embodiments, a nucleic acid construct encodes for six different fluorescent proteins, e.g., the nucleic acid construct further includes a sixth nucleic acid segment encoding a sixth fluorescent protein. In certain embodiments, a nucleic acid construct encodes for seven different fluorescent proteins, e.g., the nucleic acid construct further includes a seventh nucleic acid segment encoding a seventh fluorescent protein. In certain embodiments, a nucleic acid construct encodes for eight different fluorescent proteins, e.g., the nucleic acid construct further includes an eighth nucleic acid segment encoding an eighth fluorescent protein. In certain embodiments, a nucleic acid construct encodes for nine different fluorescent proteins, e.g., the nucleic acid construct further includes a ninth nucleic acid segment encoding a ninth fluorescent protein. In certain embodiments, a nucleic acid construct encodes for ten different fluorescent proteins, e.g., the nucleic acid construct further includes a tenth nucleic acid segment encoding a tenth fluorescent protein.

In certain embodiments, a nucleic acid construct of the present disclosure encodes a first fluorescent protein and a second fluorescent protein. In certain embodiments, a nucleic acid construct of the present disclosure encodes a first fluorescent protein, a second fluorescent protein and a third fluorescent protein. In certain embodiments, a nucleic acid construct of the present disclosure encodes a first fluorescent protein, a second fluorescent protein, a third fluorescent protein and a fourth fluorescent protein. In certain embodiments, a nucleic acid construct of the present disclosure encodes a first fluorescent protein, a second fluorescent protein, a third fluorescent protein, a fourth fluorescent protein and a fifth fluorescent protein. In certain embodiments, a nucleic acid construct of the present disclosure encodes a first fluorescent protein, a second fluorescent protein, a third fluorescent protein, a fourth fluorescent protein, a fifth fluorescent protein and a sixth fluorescent protein. In certain embodiments, a nucleic acid construct of the present disclosure encodes a first fluorescent protein, a second fluorescent protein, a third fluorescent protein, a fourth fluorescent protein, a fifth fluorescent protein, a sixth fluorescent protein and a seventh fluorescent protein. In certain embodiments, a nucleic acid construct of the present disclosure encodes a first fluorescent protein, a second fluorescent protein, a third fluorescent protein, a fourth fluorescent protein, a fifth fluorescent protein, a sixth fluorescent protein, a seventh fluorescent protein and an eighth fluorescent protein. In certain embodiments, a nucleic acid construct of the present disclosure encodes a first fluorescent protein, a second fluorescent protein, a third fluorescent protein, a fourth fluorescent protein, a fifth fluorescent protein, a sixth fluorescent protein, a seventh fluorescent protein, an eighth fluorescent protein and a ninth fluorescent protein. In certain embodiments, a nucleic acid construct of the present disclosure encodes a first fluorescent protein, a second fluorescent protein, a third fluorescent protein, a fourth fluorescent protein, a fifth fluorescent protein, a sixth fluorescent protein, a seventh fluorescent protein, an eighth fluorescent protein, a ninth fluorescent protein and a tenth fluorescent protein.

Any fluorescent protein can be encoded by a nucleic acid construct of the present disclosure. In certain embodiments, a fluorescent protein encoded by a nucleic acid construct of the present disclosure can be a green fluorescent protein (GFP), a red fluorescent protein (RFP), a blue fluorescent protein (BFP), a cyan fluorescent protein (CFP), a yellow fluorescent protein (YFP), an orange fluorescent protein (OFP), a far-red fluorescent protein, a near-infrared fluorescent protein, a long stokes shift fluorescent protein, a photo-activatable fluorescent protein, a photoconvertible fluorescent protein, a photoswitchable fluorescent protein, a timer fluorescent protein and mutants or variants thereof. For example, but not by way of limitation, a GFP can be detected with an excitation range of about 485, e.g., 488, and an emission range of about 515, e.g., 525, an RFP can be detected with an excitation range of about 580, e.g., 594, and an emission range of about 610, e.g., 620, and a BFP can be detected with an excitation range of about 400, e.g., 405, and an emission range of about 425, e.g., 450. Additional not limiting examples of fluorescent proteins include sfGFP, deGFP, eGFP, Venus, mVenus, YFP, Cerulean, Citrine, CFP, eYFP, eCFP, mRFP, mCherry, mmCherry, mTurquoise2, mKO2, mTagBFP2 and mutants or variants thereof. Further non-limiting examples of fluorescent proteins are disclosed in WO 2007/142582, which are incorporated by reference herein in their entireties.

In certain embodiments, the fluorescent proteins encoded by the nucleic acid construct are the same. In certain embodiments, each of the fluorescent proteins encoded by the nucleic acid construct are different. For example, but not by way of limitation, each of the fluorescent proteins encoded by a nucleic acid construct of the present disclosure are dependently selected from the group consisting of a green fluorescent protein (GFP), a red fluorescent protein (RFP), a blue fluorescent protein (BFP), a cyan fluorescent protein (CFP), a yellow fluorescent protein (YFP), an orange fluorescent protein (OFP), a far-red fluorescent protein, a near-infrared fluorescent protein, a long stokes shift fluorescent protein, a photo-activatable fluorescent protein, a photoconvertible fluorescent protein, a photoswitchable fluorescent protein, a timer fluorescent protein and mutants or variants thereof. In certain embodiments, one of the fluorescent proteins can be a GFP and the other fluorescent protein can be an RFP, a BFP, a CFP, a YFP, an OFP, a far-red fluorescent protein, a near-infrared fluorescent protein, a long stokes shift fluorescent protein, a photo-activatable fluorescent protein, a photoconvertible fluorescent protein, a photoswitchable fluorescent protein or a timer fluorescent protein. In certain embodiments, one of the fluorescent proteins can be an RFP and the other fluorescent protein can be a GFP, a BFP, a CFP, a YFP, an OFP, a far-red fluorescent protein, a near-infrared fluorescent protein, a long stokes shift fluorescent protein, a photo-activatable fluorescent protein, a photoconvertible fluorescent protein, a photoswitchable fluorescent protein or a timer fluorescent protein. In certain embodiments, one of the fluorescent proteins can be an BFP and the other fluorescent protein can be a GFP, an RFP, a CFP, a YFP, an OFP, a far-red fluorescent protein, a near-infrared fluorescent protein, a long stokes shift fluorescent protein, a photo-activatable fluorescent protein, a photoconvertible fluorescent protein, a photoswitchable fluorescent protein or a timer fluorescent protein. In certain embodiments, one of the fluorescent proteins can be an CFP and the other fluorescent protein can be a GFP, an RFP, a BFP, a YFP, an OFP, a far-red fluorescent protein, a near-infrared fluorescent protein, a long stokes shift fluorescent protein, a photo-activatable fluorescent protein, a photoconvertible fluorescent protein, a photoswitchable fluorescent protein or a timer fluorescent protein. In certain embodiments, one of the fluorescent proteins can be an YFP and the other fluorescent protein can be a GFP, an RFP, a BFP, a CFP, an OFP, a far-red fluorescent protein, a near-infrared fluorescent protein, a long stokes shift fluorescent protein, a photo-activatable fluorescent protein, a photoconvertible fluorescent protein, a photoswitchable fluorescent protein or a timer fluorescent protein. In certain embodiments, one of the fluorescent proteins can be an OFP and the other fluorescent protein can be a GFP, an RFP, a BFP, a YFP, an CFP, a far-red fluorescent protein, a near-infrared fluorescent protein, a long stokes shift fluorescent protein, a photo-activatable fluorescent protein, a photoconvertible fluorescent protein, a photoswitchable fluorescent protein or a timer fluorescent protein. In certain embodiments, one of the fluorescent proteins can be a far-red fluorescent protein and the other fluorescent protein can be a GFP, an RFP, a BFP, a CFP, a YFP, an OFP, a near-infrared fluorescent protein, a long stokes shift fluorescent protein, a photo-activatable fluorescent protein, a photoconvertible fluorescent protein, a photoswitchable fluorescent protein or a timer fluorescent protein. In certain embodiments, one of the fluorescent proteins can be a near-infrared fluorescent protein and the other fluorescent protein can be a GFP, an RFP, a BFP, a CFP, a YFP, an OFP, a far-red fluorescent protein, a long stokes shift fluorescent protein, a photo-activatable fluorescent protein, a photoconvertible fluorescent protein, a photoswitchable fluorescent protein or a timer fluorescent protein. In certain embodiments, one of the fluorescent proteins can be a long stokes shift fluorescent protein and the other fluorescent protein can be a GFP, an RFP, a BFP, a CFP, a YFP, an OFP, a far-red fluorescent protein, near-infrared fluorescent protein, a photo-activatable fluorescent protein, a photoconvertible fluorescent protein, a photoswitchable fluorescent protein or a timer fluorescent protein. In certain embodiments, one of the fluorescent proteins can be a photo-activatable fluorescent protein and the other fluorescent protein can be a GFP, an RFP, a BFP, a CFP, a YFP, an OFP, a far-red fluorescent protein, near-infrared fluorescent protein, a long stokes shift fluorescent protein, a photoconvertible fluorescent protein, a photoswitchable fluorescent protein or a timer fluorescent protein. In certain embodiments, one of the fluorescent proteins can be a photoconvertible fluorescent protein and the other fluorescent protein can be a GFP, an RFP, a BFP, a CFP, a YFP, an OFP, a far-red fluorescent protein, near-infrared fluorescent protein, a long stokes shift fluorescent protein, a photo-activatable fluorescent protein, a photoswitchable fluorescent protein or a timer fluorescent protein. In certain embodiments, one of the fluorescent proteins can be a photoswitchable fluorescent protein and the other fluorescent protein can be a GFP, an RFP, a BFP, a CFP, a YFP, an OFP, a far-red fluorescent protein, near-infrared fluorescent protein, a long stokes shift fluorescent protein, a photo-activatable fluorescent protein, a photoconvertible fluorescent protein or a timer fluorescent protein. In certain embodiments, one of the fluorescent proteins can be a timer fluorescent protein and the other fluorescent protein can be a GFP, an RFP, a BFP, a CFP, a YFP, an OFP, a far-red fluorescent protein, near-infrared fluorescent protein, a long stokes shift fluorescent protein, a photo-activatable fluorescent protein, a photoconvertible fluorescent protein or a photoswitchable fluorescent protein.

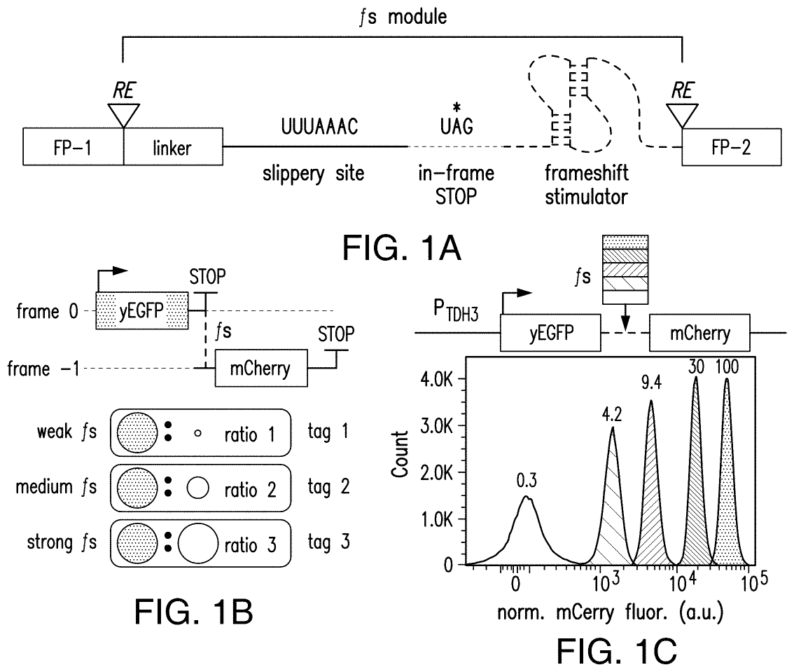

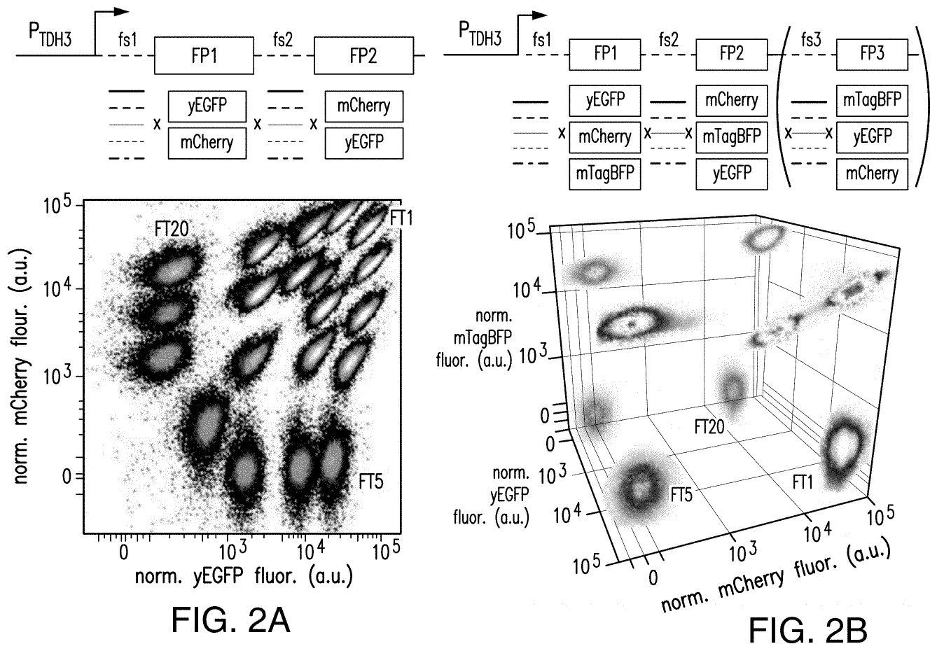

In certain embodiments, each of the fluorescent proteins encoded by a nucleic acid construct of the present disclosure are dependently selected from the group consisting of sfGFP, deGFP, eGFP, Venus, mVenus, YFP, Cerulean, Citrine, CFP, eYFP, eCFP, mRFP, mCherry, mmCherry, mTurquoise2, mKO2, mTagBFP2 and mutants or variants thereof. For example, and not by way of limitation, the first fluorescent protein can be a GFP, e.g., eGFP and the second fluorescent protein can be a RFP, e.g., mCherry. See, for example, FIG. 1 and FIG. 2. In certain embodiments, the first fluorescent protein can be a GFP, e.g., eGFP, the second fluorescent protein can be a RFP, e.g., mCherry, and the third fluorescent protein can be a BFP, e.g., mTagBFP. See, for example, FIG. 2.

In certain embodiments, the nucleic acid sequence of the fluorescent protein encoded by a nucleic construct of the present disclosure includes a sequence disclosed in Tables 6 and 7. In certain embodiments, the nucleic acid sequence of the fluorescent protein encoded by a nucleic construct of the present disclosure includes a sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence disclosed in Tables 6 and/or 7. In certain embodiments, the DNA sequence of the fluorescent protein eGFP is provided in Table 6. In certain embodiments, the DNA sequence of the fluorescent protein mCherry is provided in Table 6. In certain embodiments, the DNA sequence of the fluorescent protein mTagBFP2 is provided in Table 6. In certain embodiments, the DNA sequence of the fluorescent protein mTurquoise is provided in Table 7. In certain embodiments, the DNA sequence of the fluorescent protein mVenus is provided in Table 7. In certain embodiments, the DNA sequence of the fluorescent protein mKO2 is provided in Table 7.

In certain embodiments, a nucleic acid of the present disclosure can include one or more stop codons, e.g., two or more, three or more, four or more, five or more, six or more, seven or more, eight or more or nine or more. In certain embodiments, a stop codon is located between the nucleic acid segments that encode the fluorescent proteins. See, for example, FIG. 1. For example, but not by way of limitation, the stop codon can be located between the nucleic acid segments encoding the fluorescent proteins, e.g., between the nucleic acid segment encoding the first fluorescent protein and the nucleic acid segment encoding the second fluorescent protein. In certain embodiments, the stop codon is in frame with the fluorescent protein, e.g., first fluorescent protein, such that a ribosome translating the mRNA transcription of the construct will stop translation before reaching the second fluorescent protein if the ribosome is in frame with the first fluorescent protein. In certain embodiments, the stop codon is located between a slippery site and a frameshift stimulatory sequence. In certain embodiments, the stop codon is located 8 nucleotides downstream of the slippery sequence. In certain embodiments, the stop codon can be located downstream of the frameshift stimulatory sequence.

In certain embodiments, a nucleic acid of the present disclosure can include one or more slippery sites, e.g., two or more, three or more, four or more, five or more, six or more, seven or more, eight or more or nine or more. In certain embodiments, a slippery site, or slippery sequence, is positioned upstream of the stop codon. In certain embodiments, a slippery site, or slippery sequence, is positioned between the nucleic acid segments encoding the fluorescent proteins, e.g., between the nucleic acid segment encoding the first fluorescent protein and the nucleic acid segment encoding the second fluorescent protein. A slippery site, when translated into mRNA (and also called a slippery site as mRNA) is a sequence where the compliment tRNA can sometimes shift at least one base pair after pairing with its anticodon, resulting in a change in the reading frame for subsequent translation. In certain embodiments, the slippery site is positioned upstream of the frameshift stimulatory sequence. In certain embodiments, the slippery sequence can include from about 5 to about 20 nucleotides, e.g., from about 5 to about 15 or from about 5 to about 10 nucleotides. In certain embodiments, the slippery sequence can include about 7 nucleotides. In certain embodiments, a slippery site can include the nucleic acid sequence disclosed in FIG. 1, e.g., UUUAAAC.

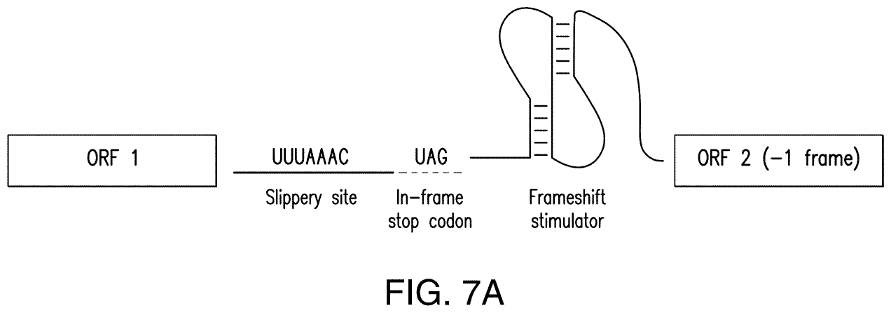

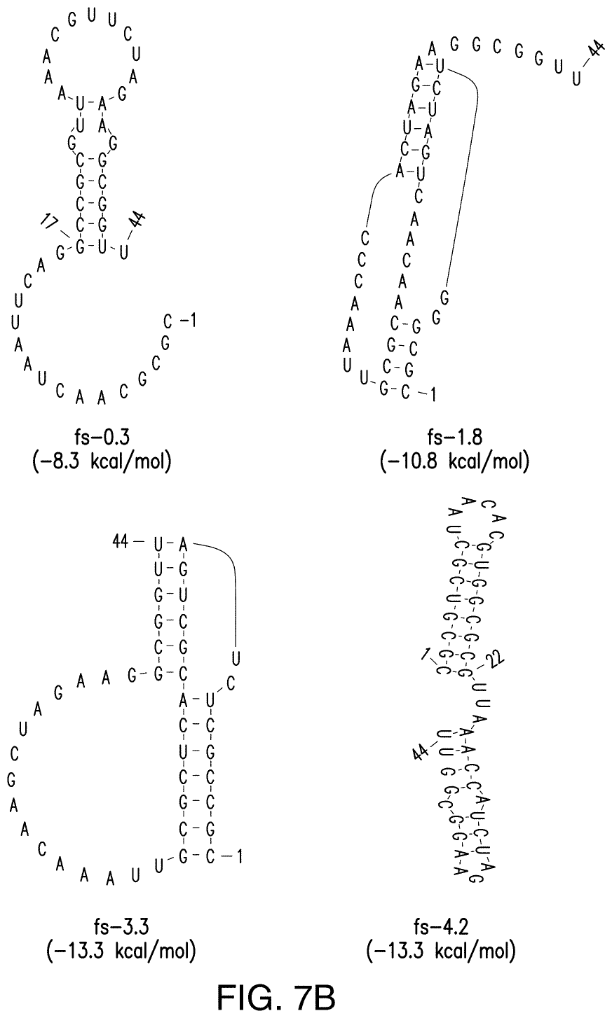

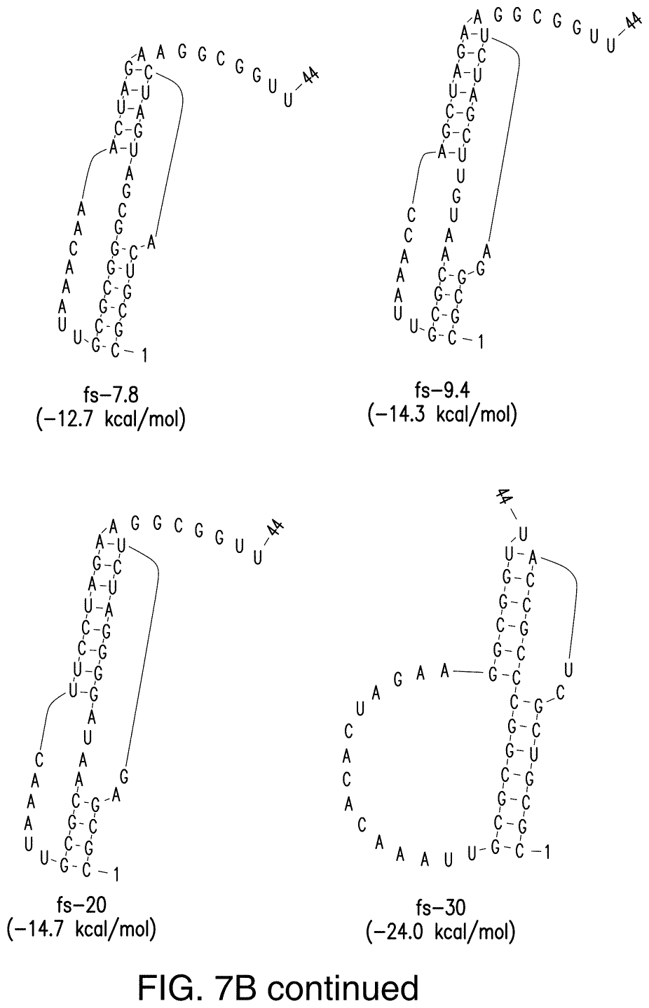

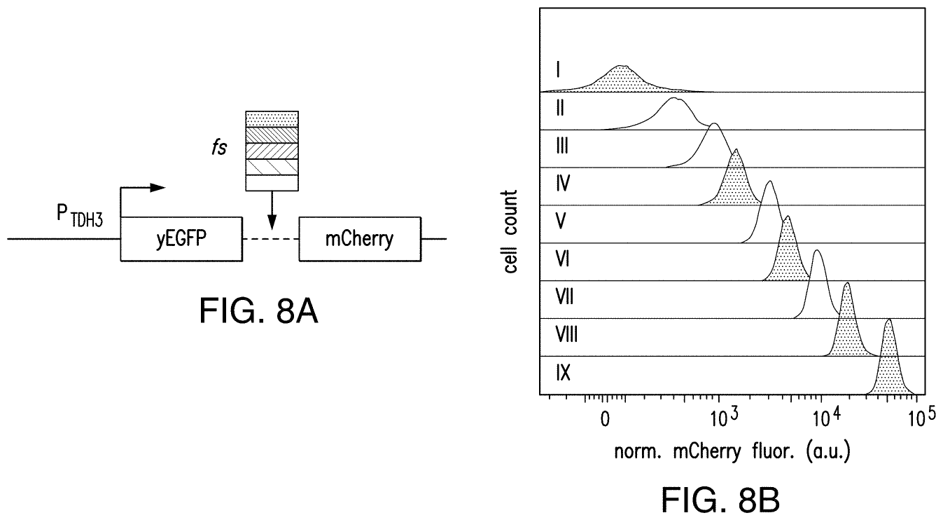

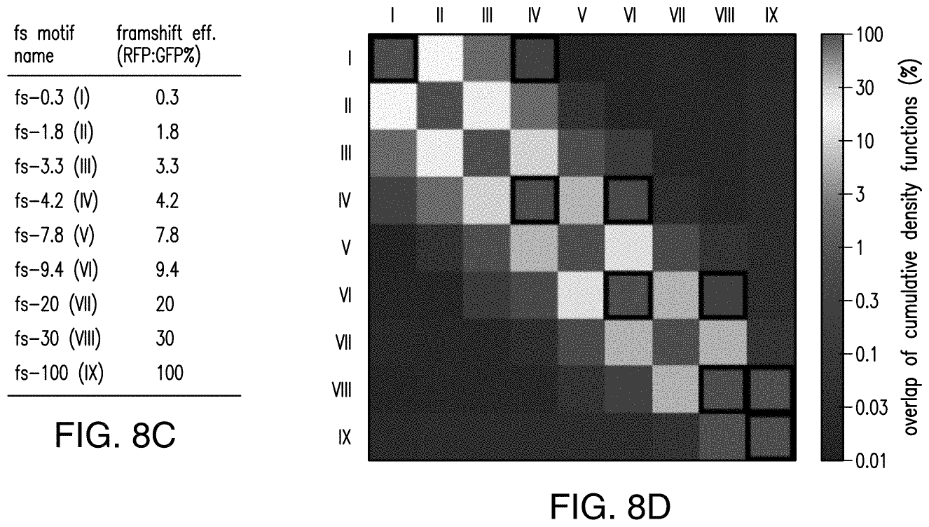

In certain embodiments, a nucleic acid of the present disclosure can include one or more frameshift stimulatory sequences, e.g., two or more, three or more, four or more, five or more, six or more, seven or more, eight or more or nine or more. For example, but not by way of limitation, a frameshift stimulatory sequence can be positioned to increase the probability of a frameshift at the slippery site. Different frameshift stimulatory sequences produce different probabilities of frameshifting (see, e.g., FIG. 8). The more often frameshifting occurs, the more often the stop codon will be out of frame, and translation will continue to the second fluorescent protein. Therefore, the ratio between the two fluorescent proteins can be controlled based on the frameshift stimulatory signal used. In certain embodiments, a frameshift stimulatory sequence is positioned between the nucleic acid segments encoding the fluorescent proteins, e.g., between the nucleic acid segment encoding the first fluorescent protein and the nucleic acid segment encoding the second fluorescent protein. For example, but not by way of limitation, a frameshift stimulatory sequence can be positioned downstream of the slippery sequence and the stop codon and upstream of the second nucleic acid segment encoding the second fluorescent protein. Alternatively, the frameshift stimulatory sequence is positioned before, e.g., upstream to, the nucleic acid segment encoding a fluorescent protein, e.g., the first fluorescent protein (see, e.g., Table 2). In certain embodiments, a frameshift stimulatory sequence can be from about 10 to about 300 nucleotides in length, e.g., from about 10 to about 275, from about 10 to about 250, from about 10 to about 225, from about 10 to about 200, from about 10 to about 175, from about 10 to about 150, from about 10 to about 125, from about 10 to about 100, from about 10 to about 90, from about 10 to about 80, from about 10 to about 70, from about 10 to about 60, from about 10 to about 50, from about 10 to about 40 nucleotide, from about 20 to about 300, from about 30 to about 300, from about 40 to about 300, from about 50 to about 300, from about 60 to about 300, from about 70 to about 300, from about 80 to about 300, from about 90 to about 300, from about 100 to about 300, from about 125 to about 300, from about 150 to about 300, from about 175 to about 300, from about 200 to about 300, from about 225 to about 300, from about 250 to about 300, from about 275 to about 300, from about 10 to about 200, from about 10 to about 100, from about 10 to about 75 or from about 20 to about 50 nucleotides in length. In certain embodiments, the frameshift stimulatory sequence is about 40 nucleotides in length. Non-limiting examples of frameshift stimulatory sequences are provided in Table 1 and FIG. 7.

In certain embodiments, a nucleic acid construct of the present disclosure includes a frameshift stimulatory sequence including a nucleic acid sequence disclosed in Table 1 and/or FIG. 7. In certain embodiments, a nucleic acid construct of the present disclosure includes a frameshift stimulatory sequence that includes a nucleic acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence disclosed in Table 1 and/or FIG. 7. In certain embodiments, the frameshift stimulatory sequence includes the nucleic acid sequence of frameshift stimulatory sequence fs-0.3 (see FIG. 7). In certain embodiments, the frameshift stimulatory sequence includes the nucleic acid sequence of frameshift stimulatory sequence fs-1.8 (see FIG. 7). In certain embodiments, the frameshift stimulatory sequence includes the nucleic acid sequence of frameshift stimulatory sequence fs-3.3 (see FIG. 7). In certain embodiments, the frameshift stimulatory sequence includes the nucleic acid sequence of frameshift stimulatory sequence fs-4.2 (see FIG. 7). In certain embodiments, the frameshift stimulatory sequence includes the nucleic acid sequence of frameshift stimulatory sequence fs-7.8 (see FIG. 7). In certain embodiments, the frameshift stimulatory sequence includes the nucleic acid sequence of frameshift stimulatory sequence fs-9.4 (see FIG. 7). In certain embodiments, the frameshift stimulatory sequence includes the nucleic acid sequence of frameshift stimulatory sequence fs-20 (see FIG. 7). In certain embodiments, the frameshift stimulatory sequence includes the nucleic acid sequence of frameshift stimulatory sequence fs-30 (see FIG. 7). In certain embodiments, the frameshift stimulatory sequence includes the nucleic acid sequence of frameshift stimulatory sequence fs-100 (see FIG. 7).

In certain embodiments, a nucleic acid construct of the present disclosure can include a first nucleic acid sequence encoding a first fluorescent protein, a slippery site, a stop codon, a frameshift stimulatory sequence and a second nucleic acid encoding a second fluorescent protein. In certain embodiments, a nucleic acid construct of the present disclosure can include a first nucleic acid sequence encoding a first fluorescent protein, a first slippery site, a first stop codon, a first frameshift stimulatory sequence, a second nucleic acid encoding a second fluorescent protein, a second slippery site, a second stop codon, a second frameshift stimulatory sequence and a third nucleic acid encoding a third fluorescent protein. Additional non-limiting examples of nucleic acid constructs, including the arrangement of the various nucleic acid segments with the nucleic acid construct, are provided in Table 2. In certain embodiments, a nucleic acid construct of the present disclosure includes a nucleotide sequence disclosed in Table 1. In certain embodiments, a nucleic acid construct of the present disclosure includes a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence disclosed in Table 1.

In certain embodiments, a nucleic acid construct of the present disclosure can include one or more linkers, e.g., two or more, three or more, four or more, five or more, six or more, seven or more, eight or more or nine or more.

The present disclosure further provides vectors and other nucleic acid molecules including the nucleic acid constructs disclosed herein. Suitable vectors include, but are not limited to, viral and non-viral vectors, plasmids, cosmids, phages, plasmids, and used for cloning, amplifying, expressing, transferring etc. of the nucleic acid sequence of the present disclosure in the appropriate target cell, e.g., yeast cell. In certain embodiments, to prepare the constructs, the partial or full-length nucleic acid construct is inserted into a vector typically by means of DNA ligase attachment to a cleaved restriction enzyme site in the vector. Alternatively, the desired nucleotide sequence can be inserted by homologous recombination in vivo, typically by attaching regions of homology to the vector on the flanks of the desired nucleotide sequence. Regions of homology are added by ligation of oligonucleotides, or by polymerase chain reaction using primers including both the region of homology and a portion of the desired nucleotide sequence, for example.

The present disclosure further provides expression cassettes or systems that can be used for the expression of the subject fluorescent proteins or for replication of the subject nucleic acid molecules. The expression cassette can exist as an extrachromosomal element or can be integrated into the genome of the cell as a result of introduction of the expression cassette into the cell. In the expression vector, a subject nucleic acid is operably linked to a regulatory sequence that can include promoters, enhancers, terminators, operators, repressors and inducers. For example, but not by way of limitation, the promoter can be for use in the cell transformed with the expression cassette, e.g., a yeast cell. Methods for preparing expression cassettes or systems capable of expressing the desired product are known for a person skilled in the art.

The present disclosure also relates to a nucleic acid construct library, consisting of multiple constructs as described above. In certain embodiments, each construct of the library can have nucleic acid segments, e.g., DNA segments, encoding a different frameshift stimulatory sequences or different combinations of frameshift stimulatory sequences, such that each construct causes cells transformed with the construct to express different ratios of fluorescent proteins. In certain embodiments, different constructs can also cause transformed cells to express different absolute amounts of the fluorescent proteins. Different nucleic acid constructs can cause the expression of different fluorescent proteins, or the same fluorescent proteins.

In certain embodiments, the nucleic acid construct library can include from about 2 to about 10,000 nucleic acid constructs. For example, by not by way of limitation, the nucleic acid construct library can include about 2 or more, about 5 or more about 10 or more, about 20 or more, about 30 or more, about 40 or more, about 50 or more, about 60 or more, about 70 or more, about 80 or more, about 90 or more, about 100 or more, about 1,000 or more, about 5,000 or more about 10,000 or more nucleic acid constructs.

### III. Cells

Cells for use in the present disclosure can be prokaryotic or eukaryotic cells. For example, but not by way of limitation, one or more nucleic acid constructs of the present disclosure can be introduced into a cell, e.g., a prokaryotic or eukaryotic cell, to generate a genetically-engineered cell.

In certain embodiments, a nucleic acid construct of the present disclosure can be introduced into a metazoan cell, a plant cell or a fungal cell. In certain embodiments, the cell can be a metazoan cell, e.g., mammalian cell. In certain embodiments, the cell can be a mammalian cell, e.g., a genetically engineered mammalian cell, e.g., a human cell or derived from a human cell. In certain embodiments, the cell can be a plant cell, e.g., a genetically engineered plant cell. In certain embodiments, the cell can be a fungal cell, e.g., a genetically engineered fungal cell.

In certain embodiments, the cell can be a genetically engineered fungal cell, e.g., a cell of Alternaria brasicicola, Arthrobotrys oligospora, Ashbya aceri, Ashbya gossypii, Aspergillus clavatus, Aspergillus flavus, Aspergillus fumigate, Aspergillus kawachii, Aspergillus nidulans, Aspergillus niger, Aspergillus oryzae, Aspergillus ruber, Aspergillus terreus, Baudoinia compniacensis, Beauveria bassiana, Botryosphaeria parva, Botrytis cinereal, Candida albicans, Candida dubliniensis, Candida glabrata, Candida guilliermondii, Candida lusitaniae, Candida parapsilosis, Candida tenuis, Candida tropicalis, Capronia coronate, Capronia epimyces, Chaetomium globosum, Chaetomium thermophilum, Chryphonectria parasitica, Claviceps purpurea, Coccidioides immitis, Colletotrichum gloeosporioides, Coniosporium apollinis, Dactylellina haptotyla, Debaryomyces hansenii, Endocarpon pusillum, Eremothecium cymbalariae, Fusarium oxysporum, Fusarium pseudograminearum, Gaeumannomyces graminis, Geotrichum candidum, Gibberella fujikuroi, Gibberella moniliformis, Gibberella zeae, Glarea lozoyensis, Grosmannia clavigera, Kazachstania Africana, Kazachstania naganishii, Kluyveromyces lactis, Kluyveromyces marxianus, Kluyveromyces waltii, Komagataella pastoris, Kuraishia capsulate, Lachancea kluyveri, Lachancea thermotolerans, Lodderomyces elongisporus, Magnaporthe oryzae, Magnaporthe poae, Marssonina brunnea, Metarhizium acridum, Metarhizium anisopliae, Mycosphaerella graminicola, Mycosphaerella pini, Nectria haematococca, Neosartorya fischeri, Neurospora crassa, Neurospora tetrasperma, Ogataea parapolymorpha, Ophiostoma piceae, Paracoccidioides lutzii, Penicillium chrysogenum, Penicillium digitatum, Penicillium oxalicum, Penicillium roqueforti, Phaeosphaeria nodorum, Pichia sorbitophila, Podospora anserine, Pseudogymnoascus destructans, Pyrenophora teres f teres, Pyrenophora tritici-repentis, Saccharomyces bayanus, Saccharomyces castellii, Saccharomyces cerevisiae, Saccharomyces dairenensis, Saccharomyces mikatae, Saccharomyces paradoxis, Scheffersomyces stipites, Schizosaccharomyces japonicus, Schizosaccharomyces octosporus, Schizosaccharomyces pombe, Sclerotinia borealis, Sclerotinia sclerotiorum, Sordaria macrospora, Sporothrix schenckii, Tetrapisispora blattae, Tetrapisispora phaffii, Thielavia heterothallica, Togninia minima, Torulaspora delbrueckii, Trichoderma atroviridis, Trichoderma jecorina, Trichoderma vixens, Tuber melanosporum, Vanderwaltozyma polyspora 1, Vanderwaltozyma polyspora 2, Verticillium alfalfae, Verticillium dahliae, Wickerhamomyces ciferrii, Yarrowia hpolytica, Zygosaccharomyces bailii, Zygosaccharomyces rouxii and combinations thereof.

In certain embodiments, the cell can be a fungal cell, e.g., a genetically engineered fungal cell, of the phylum Ascomycota. In certain embodiments, the cell, e.g., a genetically engineered cell, can be a species selected from Saccharomyces cerevisiae, Saccharomyces castellii, Saccharomyces var boulardii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipites, Komagataella (Pichia) pastoris, Candida (Pichia) guilliermondii, Candida parapsilosis, Candida auris, Yarrowia hpolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinereal, Beauvaria bassiana, Neurospora crassa, Sporothrix scheckii, Magnaporthe oryzea, Dactylellina haptotyla, Fusarium graminearum, and Capronia coronata. In certain embodiments, the one or more cell of the present disclosure is a yeast cell, e.g., Saccharomyces cerevisiae.

In certain embodiments, the present disclosure provides for genetically engineered cells including one or more nucleic acid constructs disclosed herein. For example, but not by way of limitation, a genetically engineered cell of the present disclosure can include two or more, three or more, four or more or five or more nucleic acid constructs. In certain embodiments, a genetically engineered cell of the present disclosure includes one nucleic acid construct. In certain embodiments, a genetically engineered cell of the present disclosure includes two nucleic acid constructs. In certain embodiments, a genetically engineered cell of the present disclosure includes three nucleic acid constructs. In certain embodiments, a genetically engineered cell of the present disclosure includes four nucleic acid constructs. In certain embodiments, a genetically engineered cell of the present disclosure includes five nucleic acid constructs. In certain embodiments, the two or more nucleic acid constructs can be different. Alternatively, the two or more nucleic acid constructs can be the same.

The cells to be used in the present disclosure can be genetically engineered using recombinant techniques known to those of ordinary skill in the art. Production and manipulation of the nucleic acid constructs described herein are within the skill in the art and can be carried out according to recombinant techniques described, for example, in Sambrook et al. 1989. Molecular Cloning: A Laboratory Manual, 2d ed., Cold Spring Harbor Laboratory Press, Cold Spring Harbor, N.Y. and Innis et al. (eds). 1995. PCR Strategies, Academic Press, Inc., San Diego. For example, but not by way of limitation, a cell, e.g., a yeast cell, can be genetically engineered to include one or more nucleic acid constructs of the present disclosure.

One or more endogenous genes of the genetically modified cells can be knocked out by a genetic engineering system. Various genetic engineering systems known in the art can be used. Non-limiting examples of such systems include the Clustered regularly-interspaced short palindromic repeats (CRISPR)/Cas system, the zinc-finger nuclease (ZFN) system, the transcription activator-like effector nuclease (TALEN) system, use of yeast endogenous homologous recombination and the use of interfering RNAs.

In certain embodiments, nucleic acid constructs of the present disclosure can be introduced into the yeast cell either as a construct or a plasmid. In certain embodiments, a nucleic acid can include one or more regulatory regions such as promoters, transcription factor binding sites, operators, activator binding sites, repressor binding sites, enhancers, protein-protein binding domains, RNA binding domains, DNA binding domains, and other control elements known to a person skilled in the art. For example, but not by way of limitation, a nucleic acid construct of the present disclosure can be introduced into the yeast cell either as a construct or a plasmid in which it is operably linked to a promoter active in the yeast cell or such that it is inserted into the yeast cell genome at a location where it is operably linked to a suitable promoter.

Non-limiting examples of suitable yeast promoters for inclusion in the nucleic acid constructs of the present disclosure include, but are not limited to, constitutive promoters pTef1, pPgk1, pCyc1, pAdh1, pKex1, pTdh3, pTpi1, pPyk1 and pHxt7 and inducible promoters pGal1, pCup1, pMet15, pFig1 and pFus1, GAP, P GCW14 and variants thereof. In certain embodiments, a variant of Tef1 is scTef1. In certain embodiments, the sequence of the promoter can include a nucleic acid sequence disclosed in Table 6. For example, but not by way of limitation, a nucleic acid construct of the present disclosure can include a constitutively active promoter, e.g., pTdh3, located upstream of the nucleic acid segment encoding the first fluorescent protein (see, e.g., Table 2). In certain embodiments, a nucleic acid construct can include an inducible promoter, e.g., pFus1 or pFig1, located upstream of the nucleic acid encoding the first fluorescent protein. In certain embodiments, a nucleic acid construct can include a constitutively active promoter, e.g., pAdh1, located 5′ to the nucleic acid encoding the first fluorescent protein.

In certain embodiments, nucleic acid constructs of the present disclosure can be inserted into the genome of the cell, e.g., yeast cell. In certain embodiments, one or more nucleic acid constructs can be inserted into the genome of the cell, e.g., yeast cell. In certain embodiments, two or more nucleic acid constructs can be inserted into the genome of the cell, e.g., yeast cell. In certain embodiments, three or more nucleic acid constructs can be inserted into the genome of the cell, e.g., yeast cell. In certain embodiments, four or more nucleic acid constructs can be inserted into the genome of the cell, e.g., yeast cell. In certain embodiments, five or more nucleic acid constructs can be inserted into the genome of the cell, e.g., yeast cell. For example, but not by way of limitation, one or more nucleic acid constructs of the present disclosure, e.g., can be inserted into the Ste2, Ste3 and/or HO locus of the cell. In certain embodiments, one or more nucleic acid constructs of the present disclosure can be inserted into the LEU2 locus of the cell. In certain embodiments, the one or more nucleic acids can be inserted into one or more loci that minimally affects the cell, e.g., in an intergenic locus or a gene that is not essential and/or does not affect growth, proliferation and cell signaling.

In certain embodiments, expression of a nucleic acid construct of the present disclosure in a cell results in the differential expression of the two or more fluorescent proteins. For example, but not by way of limitation, expression of a nucleic acid construct of the present disclosure encoding two different fluorescent proteins in a cell can result in the differential expression of the two or more fluorescent proteins. In certain embodiments, the genetically-engineered cell can express the first fluorescent protein and the second fluorescent protein in a ratio. For example, but not by way of limitation, the first fluorescent protein is expressed at a ratio of about 1,000:1 to about 1:1,000, e.g., from about 900:1 to about 1:900, from about 800:1 to about 1:800, from about 700:1 to about 1:700, from about 600:1 to about 1:600, from about 500:1 to about 1:500, from about 400:1 to about 1:400, from about 300:1 to about 1:300, from about 200:1 to about 1:200, from about 100:1 to about 1:100, from about 90:1 to about 1:90, from about 80:1 to about 1:80, from about 70:1 to about 1:70, from about 60:1 to about 1:60, from about 50:1 to about 1:50, from about 40:1 to about 1:40, from about 30:1 to about 1:30, from about 20:1 to about 1:20, from about 10:1 to about 1:10, from about 10:1 to about 1:100, from about 10:1 to about 1:50, from about 50:1 to about 1:100 or from about 100:1 to about 1:500 of the second fluorescent protein (see, e.g., FIG. 8). In certain embodiments, the genetically-engineered cell can express the first fluorescent protein and the third fluorescent protein in a ratio, e.g., the first fluorescent protein is expressed at a ratio of about 1,000:1 to about 1:1,000, e.g., from about 10:1 to about 1:10, of the third fluorescent protein. In certain embodiments, the genetically-engineered cell can express the second fluorescent protein and the third fluorescent protein in a ratio, e.g., the second fluorescent protein is expressed at a ratio of about 1,000:1 to about 1:1,000, e.g., from about 10:1 to about 1:10, of the third fluorescent protein.

The present disclosure also provides a cell library. In certain embodiments, a cell library of the present disclosure includes a population of cells transformed with a number of different constructs as described above. In certain embodiments, the population of cells in a cell library will express a variety of different ratios of fluorescent proteins.

In certain embodiments, the present disclosure provides a population of cells that includes one or more cells that contain one or more nucleic acid constructs disclosed herein. In certain embodiments, the cell population includes one cell type. In certain embodiments, the cell population includes one or more cell types, e.g., two or more cell types, three or more cell types, four or more cell types, five or more cell types or six or more cell types. In certain embodiment, each cell type includes a different nucleic acid construct. For example, but not by way of limitation, the present disclosure provides a cell population that include one cell type or cell that include a first nucleic acid construct. In certain embodiments, the cell population further includes a second cell type or cell that includes a second nucleic acid construct. In certain embodiments, the first nucleic acid construct and the second nucleic acid construct are different, e.g., include different frameshift modulatory sequences.

In certain embodiments, one or more genetically engineered cells of the present disclosure can be culture in a media that has a reduction in the amount and/or level of compounds, chemicals, nutrients and/or components that are photosensitive.

### IV. Methods of Use

The present disclosure further provides methods for using the nucleic acid constructs and/or the cells including the disclosed nucleic acid constructs. For example, but not by way of limitation, nucleic acid constructs of the present disclosure can be used to label cells, e.g., to distinguish between two or more cells within a population of cells. In certain embodiments, the cell population includes bacterial cells. In certain embodiments, the cell population includes fungal, e.g., yeast, cells. In certain embodiments, the cell population includes mammalian cells.

In certain embodiments, the present disclosure provides methods for labeling cells. In certain embodiments, the method can include introducing one or more nucleic acid constructs disclosed herein into the cell. In certain embodiments, the method can further include analyzing the ratio of fluorescent between the fluorescent proteins encoded by the nucleic acid construct.

In certain embodiments, the present disclosure further provides methods for distinguishing between two or more cells within a population of cells. For example, but not by way of limitation, a method of the present disclosure includes introducing one or more disclosed nucleic constructs that encode two or more fluorescent proteins into a cell to generate a genetically engineered cell. In certain embodiments, the method can further include determining the expression level of the two or more fluorescent proteins into the cell, e.g., the expression level of the first fluorescent protein and the second fluorescent protein. In certain embodiments, the method can further include comparing the expression levels of the two or more fluorescent proteins into the cell, e.g., the expression level of the first fluorescent protein and the second fluorescent protein, to determine the ratio of the expression level of the first fluorescent protein and the second fluorescent protein. In certain embodiments, the method can further include identifying the cells with a particular ratio of the first fluorescent protein expression level to the expression level of the second fluorescent protein. In certain embodiments, the method can further include determining the expression level of a third, fourth, fifth, sixth, seventh, eighth, night or tenth fluorescent protein and comparing it to the expression level of a different fluorescent protein expressed in the cell. In certain embodiments, the genetically engineered cell expresses the first fluorescent protein and the second fluorescent protein in a ratio that allows it to be distinguished from other cells within the cell population, e.g., cells that express the first fluorescent protein and the second fluorescent protein in a different ratio. The ratio at which the two different fluorescent proteins will be expressed depends on the nucleic acid construct that is present within the cell and such cells can be identified based on this ratio.

In certain embodiments, the present disclosure further provides methods for labeling cell free extracts.

In certain embodiments, the expression level of the fluorescent proteins can be detected by any technique known in the art. In certain embodiments, the expression level can be determined by flow cytometry. In certain embodiments, the expression level can be determined by fluorescence microscopy.

In certain embodiments, the nucleic acid constructs can be used to track cells with a mixed cell population, e.g., a cell population of different cell types. For example, but not by way of limitation, the nucleic acid constructs can be used to characterize a population of cells over time. In certain embodiments, one type of cell within the mixed cell population can include one nucleic acid construct and a different type of cell within the mixed population of cells can include a different nucleic acid construct. In certain embodiments, the mixed population of cells can be monitored over time and identified by the expression of the nucleic acid constructs, e.g., by analyzing the ratio of the fluorescent proteins expressed within the various cells within the mixed population.

In certain embodiments, the method for distinguishing between two or more cells within a population of cells can be performed by a computer that has at least a processor and at least a memory the processor can access. In certain embodiments, the method includes collecting the fluorescence data of the first fluorescent protein, which is a function of the number of cells and the intensity of that fluorescence. In certain embodiments, the method can further include collecting the fluorescence data of the second fluorescent protein, which is a function of the number of cells and the intensity of that fluorescence. In certain embodiments, the method can include collecting the sum fluorescence data, which is a function of the number of cells and the sum of the first and second proteins' fluorescence. In certain embodiments, the method can include collecting the ratio fluorescence data, which is a function of the number of cells and the ratio of the first protein's fluorescence to the second protein's fluorescence. In certain embodiments, the method includes creating a set of graph bisections that will be used to bisect each of the graphs. The set of graph bisections identifies the location of the bisection, where the location can be identified as a local minimum. In certain embodiments, the set of graph bisections can identify more than one bisection location in a graph. In certain embodiments, the set of graph bisections can be specific to a set of constructs. In certain embodiments, the bisection divides the graph into two equal halves. Alternatively, the bisection divides the graph into two unequal halves. A person having ordinary skill in the art will realize that one half, in this context, means one of the two portions of the graph created when the graph is divided by the bisection. In certain embodiments, the method also includes bisecting a graph of the first protein fluorescence on one axis and the number of cells on the second axis according to the set of graph bisections, where half of the bisected data is temporarily discarded, while the other half is retained. In certain embodiments, the method also includes bisecting a graph of the second protein fluorescence on one axis and the number of cells on the second axis to the set of graph bisections, where half of the bisected data is temporarily discarded, while the other half is retained. In certain embodiments, the method includes bisecting a graph of the sum of the first protein fluorescence and the second protein fluorescence on one axis and the number of cells on the second axis to the set of graph bisections, where half of the bisected data is temporarily discarded, while the other half is retained. In certain embodiments, the method also includes bisecting a graph of the ratio of the first protein's fluorescence to the second protein's fluorescence on one axis and the number of cells on the second axis to the set of graph bisections, where half of the bisected data is temporarily discarded, while the other half is retained. The graphs are bisected as described above until all bisections in the set of bisections are performed. This results in only a single peak in each graph, representing a population of cells with the same first fluorescent protein expression and the same second fluorescent protein expression. A computer can be used to execute some of these steps, or all of these steps.

### V. Kits

The present disclosure further provides kits for generating the genetically engineered cells described herein. For example, a kit of the present disclosure includes one or more nucleic acid constructs described herein, e.g., to generate a genetically engineered cell.

In certain embodiments, a kit of the present disclosure includes a container including one or more nucleic acid constructs. In certain embodiments, the nucleic acid construct includes a first nucleic acid segment encoding a first fluorescent protein, a slippery site, a stop codon, a frameshift stimulatory sequence and a second nucleic acid segment encoding a second fluorescent protein. Non-limiting examples of nucleic acid constructs is provided in Table 2. In certain embodiments, the kit can further include a second container containing one or more cells that can be transformed with the nucleic acid constructs provided in the first container.

In certain embodiments, a kit of the present disclosure can include a container including at least one or more genetically-engineered cells described herein. In certain embodiments, the one or more genetically-engineered cells include one or more nucleic acid constructs. In certain embodiments, a nucleic acid construct of the present disclosure encodes a first fluorescent protein and a second fluorescent protein.

### EXAMPLES

The presently disclosed subject matter will be better understood by reference to the following Examples, which are provided as exemplary of the presently disclosed subject matter, and not by way of limitation.

Recognizing the limitations of the prior art, a genetically encoded fluorescent cell barcoding technology that preserves all of the advantages of fluorescent proteins (FPs) while also delivering a large set of robust, unique and well-defined tags was established. Moreover, this was achieved by using a minimal number of fluorescence channels. To this end one embodiment developed was a palette of ratiometric fluorescent barcodes, referred to as FRAME-tags (Frameshift-controlled RAtiometric Multi-fluorescent protein Expression tags). FRAME-tags are distinguished based on absolute FP expression ratios directly encoded in their mRNAs as seen in FIG. 1.

The FRAME-tag is a scalable palette of genetically encoded barcodes that achieves 20+ resolvable cell markers using two or more FPs. FRAME-tags can be a nucleic acid, e.g., DNA, construct and encode single-mRNA constructs that leverage custom ribosomal frameshifting RNA modules (frameshift modules, or frameshift motifs) to precisely control FP synthesis ratios leading to narrow fluorescence distributions that are robust to biological noise. This allows FP ratios to be used as the barcode signal for cell tracking. FRAME-tags can be used to accurately identify cells in high throughput using both flow cytometry and fluorescence microscopy.

The present disclosure uses a co-translational mechanism that can precisely regulate FP synthesis ratios from a single mRNA. In particular, a −1 programmed ribosomal frameshifting (—1 PRF) was identified as a mechanism to encode FP stoichiometry since it uses self-contained RNA elements, is active in a wide variety of organisms, and can be tuned by the choice of frameshift stimulatory RNA signals (also called a frameshift stimulator or stimulatory motif). Previously, a large collection of −1 PRF signals that possess frameshifting efficiencies spanning two orders of magnitude in yeast (0.2% to 30%) was reported. In this example, discrete frameshift modules (fs) were designed that incorporate these −1 PRF frameshift stimulatory signals for modular assembly of a FRAME-tag palette (FIG. 1A and FIG. 7). fs modules can be designed so that a failure to frameshift results in immediate translation termination at a proximal stop codon between two FPs, whereas frameshifting allows for continued translation in the −1 reading frame. This leads to fs-defined ratios of frameshift to non-frameshift FP products (FIG. 1B).

Frameshift module architecture and predicted frameshift stimulatory signals are shown in FIG. 7. FIG. 7A shows open reading frames (ORFs) that are separated by frameshift modules that contain a heptanucleotide slippery sequence, an in frame amber stop codon, and a downstream frameshift stimulatory signal. Frameshifting at the slippery site is stimulated by adjacent RNA secondary structures in cis, called frameshift stimulatory signals, such as hairpins and pseudoknots (shown in FIG. 7B), which divert translation to the −1 reading frame (UUU-AAA-CUA . . . ) containing ORF2. The structure of these frameshift stimulatory signals can be predicted (FIG. 7B). While the in frame amber stop codon is shown between the slippery site and frameshift stimulatory signal, the stop codon could be positioned after the frameshift stimulatory signal as well.

Frameshift Vs) modules are designed to control the stoichiometry of upstream and downstream open reading frames (FP-1 and FP-2) via −1 PRF (FIG. 1A). They contain a linker, a tRNA slippery site, an in-frame stop codon and a custom frameshift stimulatory signal. fs modules are flanked by restriction sites (RE) for convenient cloning (FIG. 1B). At the fs modules, translation either terminates or continues in the −1 reading frame. Distinct ratios of the upstream and downstream proteins (yEGFP and mCherry, in this example) can be produced based on fs module frameshift efficiency. These ratios can be used to uniquely tag cells (FIG. 1C). Frameshift modules can be placed in front of any protein, e.g., FP, to control that protein's absolute expression level. This means a fs module can be placed in front of both fluorescent proteins to control the absolute expression of both proteins. These absolute expression levels and relative ratios can be used to uniquely tag cells.

While FRAME-tags use fs modules to control the expression ratio of two fluorescent proteins, the same construct architecture shown in FIG. 7A—ORFs separated by a fs module—can be used to control the expression ratio between any two proteins. Thus, a construct can be created that, after transformation, causes a transformed cell to express any two proteins in a predetermined ratio, the ratio being a function of the fs module.

FRAME-tags can be used to track mixed cell populations over time, allowing for characterization of population-wide rates of change in phenotype and composition. Recently, DNA recording systems have been developed to track cell lineage and expression intensity. The limit on time-resolution for FRAME-tag data is dictated only by the rate of fluorescence capture, and acquired data is relevant at both the population and single cell scale. Furthermore, time-resolved data is obtained directly from samples without any further manipulations or reagents, whereas sequencing-based methods require multiple labor- and reagent-intensive steps that delay data recovery. Because of this fast acquisition time, FRAME-tags enable rapid experimental iteration and open the door to continuous monitoring using automated flow cytometry and microscopy.

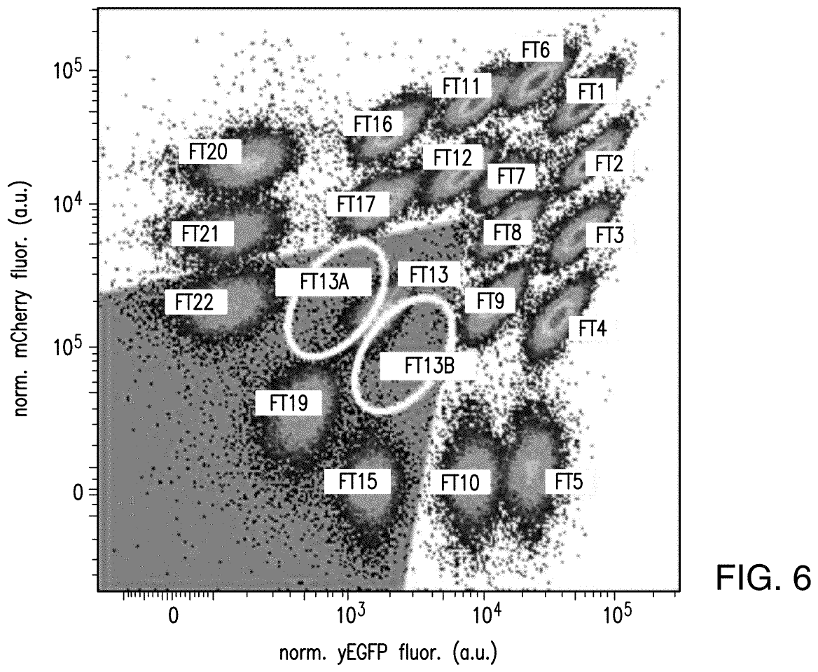

Extrapolating from the FRAME-tags (FTs) that were constructed and tested empirically, the expected number of resolvable FT variants in three-color space can be predicted (FIG. 6). Some of the FT constructs would likely require −1 PRF signals that were not implemented for the main two-color system in order to fit additional FTs that produce the desired absolute expression level and ratios of FPs for the three-color system. First, in the vicinity of FT-13, there should be sufficient room to add other FTs, or replace FT-13 with two new FTs. Here, it is a reasonable assumption that such an exchange can be made with available fs modules and an additional FT added to the existing two-FP arrangement. Also, three-color FP variants will likely behave in a similar manner to the two-color constructs in terms of absolute protein expression and population distributions, as this was observed for the 3-FP constructs generated. By the same logic, it can be possible to make three, four, five, or any number-color FP variants.

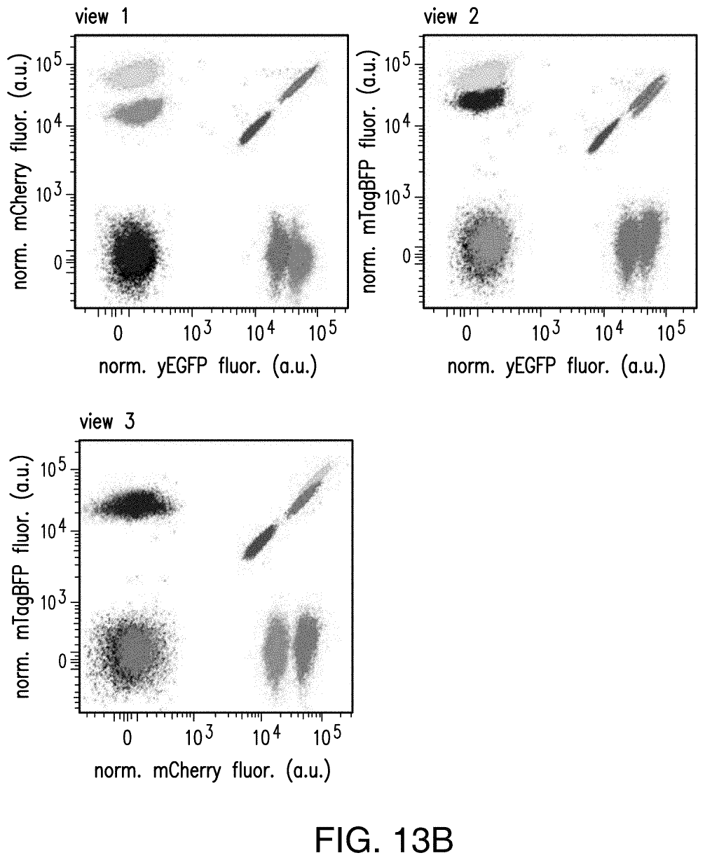

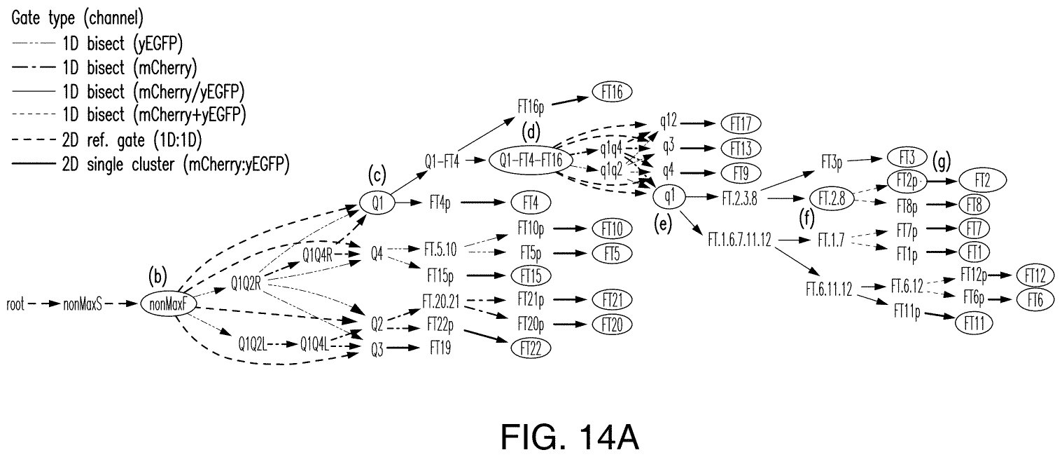

As shown in FIG. 2B and FIG. 13, the three-color space can be represented as a three-dimensional cube that contains the FRAME-tag palette. An additional FT can be added to the two-color set to create a palette of 21 FTs as shown below (FIG. 6, replacing FT-13 with FT-13A and FT-13B, red circles). Because of differences in resolution between FTs that have high vs. low overall FP expression, scaling in a new dimension cannot be achieved by multiplying the existing set by a constant. Rather, scalability is assumed in the third color dimension to follow rules of scalability in the two-dimension case. Constructs that express at least one FP at the 30% or 100% absolute level are capable of being scaled across the second color dimension so that a total of 5 FTs is created. Among the set of 21 hypothetical 2-dimensional FTs, the 16 shown in the orange zone (FT-1, FT-2, FT-3, FT-4, FT-5, FT-6, FT-7, FT-8, FT-9, FT-10, FT-11, FT-12, FT-16, FT-17, FT-20, and FT-21) satisfy this condition. So, it is predicted that each of these 16 FTs can generate 4 additional resolvable FTs each using the new color dimension for a total of 80 three-color FTs (16×5). FTs that do not express at least one FP at the 30% or 100% level (FT-13A, FT-13B, FT-15, FT-19, FT-22) shown in the blue zone can be scaled in the third color dimension to create only 3 additional resolvable FTs each, thus generating a total of 20 total FTs (5×4). Summing these, it is predicted that a total of 100 FTs can be generated based on this simple scaling analysis.

While the raw cytometry data generated by flow cytometry or microscopy using FRAME-tagged cells (FTs) can be analyzed manually using available software, this process is tedious for samples that include many FT populations and, as with any manually analyzed cytometry data, it is subjective. Therefore, to simplify analysis of FTs and to remove user bias, a method implemented was designed as a front end for the existing suite of automated flow cytometry processing functions collected under the R package openCyto. Thus, this package runs on a computer and identifies different FT populations based on their particular FRAME-tag. The computer has both a processor and memory that is accessible to the processor. The method can be loaded into memory. The processor and memory can be in communication with the flow cytometer or the fluorescence microscope. The communication can be wireless or wired.

The existing openCyto pipeline enables automated gating of cytometry data based on a user-defined input file (the “Gating Template”) that defines a gating hierarchy and the corresponding automatic gating functions to use at each level of the hierarchy. The instant method builds on this pipeline in three ways: (1) it dynamically generates the correct “Gating Template” based on a much simpler input file that need only list the FTs present in the sample; (2) it automatically pre-processes the raw data to generate two derived parameters used in gating (RFP/GFP ratio and RFP+GFP total fluorescence); and (3) it includes an automatic gating function that simplifies valley-specific bisection of 1-dimensional histograms with multiple sharp peaks and valleys characteristic of the FT populations.

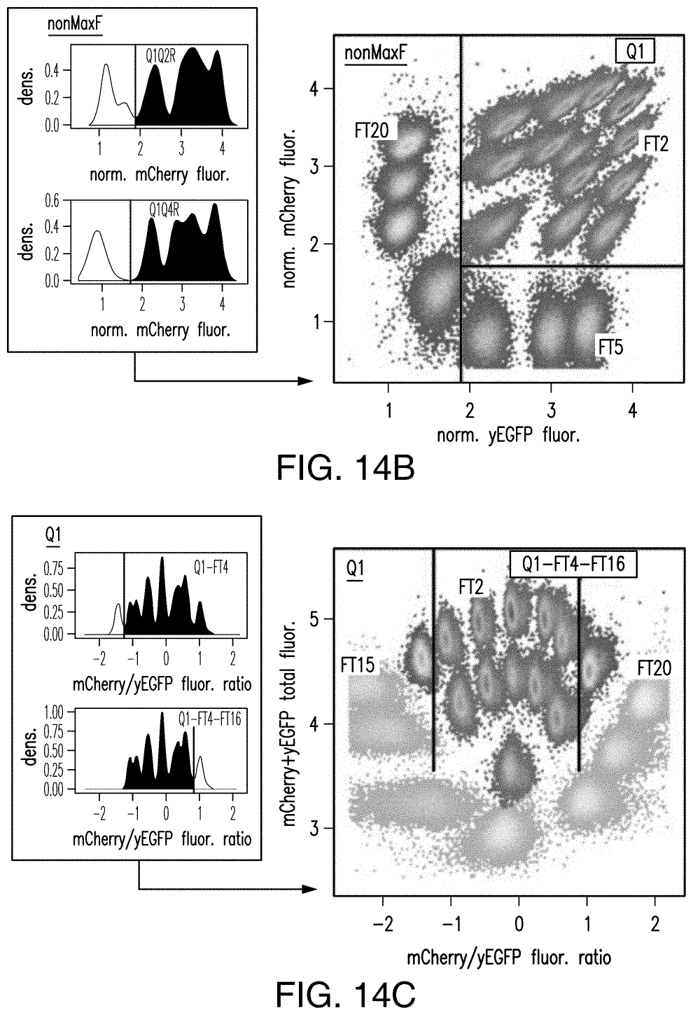

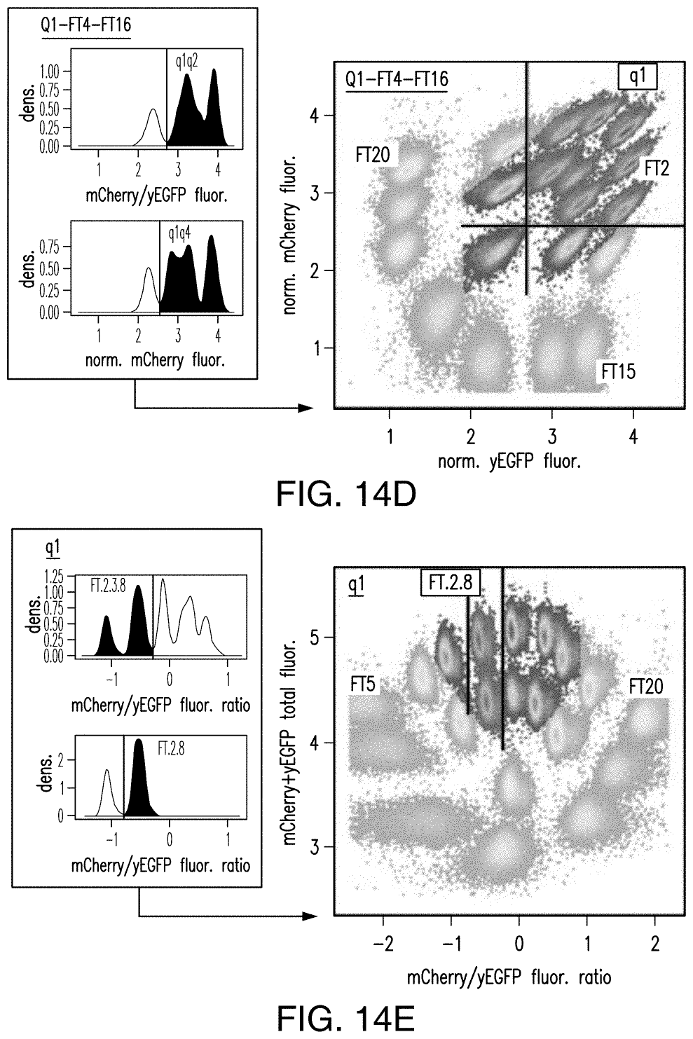

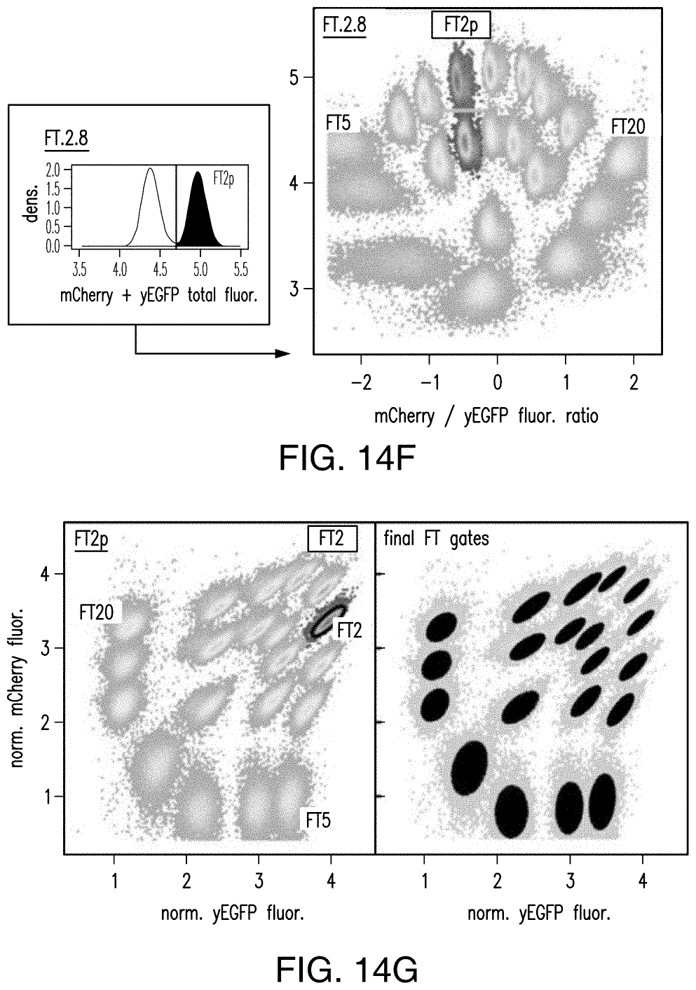

This method (FIG. 14) reads the fluorescence data from at least two fluorophores and processes it into a series of histograms; a histogram for the first fluorophore (FIG. 14B upper histogram), a histogram for the second fluorophore (FIG. 14B lower histogram), a histogram for the first fluorophore/second fluorophore ratio (FIG. 14C upper and lower histogram), and a histogram for the first fluorophore+second fluorophore (FIG. 14F histogram). Each of these histograms can then be bisected (for example FIG. 14B upper histogram, green line; or FIG. 14A, each green, red, blue, or magenta line), with one half of the data being set aside (FIG. 14B upper histogram, light grey portion of the curve) and the other half being subject to another bisection (FIG. 14B upper histogram, black portion of the curve). The bisection does not necessarily divide the graph into equal halves. The set of graph bisections can be determined in advance for a group of FRAME-tags because the FRAME-tags produce a consistent cluster structure. The last peak remaining is identified as a particular FT population (FIG. 14G), and the process repeats using the set-aside data until all peaks are identified.

This method also enables robust and efficient assignment of FT indices to each event even for high throughput and real-time experiments that can generate hundreds of individual data files. This method can leverage the consistent cluster structure of FTs. This cluster structure can be completely predetermined based on the known FTs in the sample and therefore efficiently traversed computationally. However, this method can still be applied to a cluster structure de novo.

FRAME-tags efficiently minimize the number of fluorescent channels that are required for barcode identification, thus allowing other orthogonal fluorescent reporters to be used alongside FRAME-tags. These reporters can be analyzed in multiplex through FRAME-tag-indexed deconvolution of the reporters' bulk fluorescent signals. Using a promoter driven orthogonal FP, expression from 20 yeast promoters across 21 experimental conditions in multiplex were profiled using FRAME-tags as promoter identifiers. Beyond promoter activity, FRAME-tags could also be used to multiplex other fluorescent reporters such as those used for detection of calcium levels, phosphorylation state and receptor signaling. FRAME-tags do not suffer signal dilution over time. Therefore, in conjunction with other fluorescent reporters, FRAME-tags can be applicable to various multiplexed phenotypic screens including microbial expression profiling, cell state reporters, and drug screening.

As developed here, FRAME-tags can be useful for wide range of applications since they are modular, scalable, and can be conveniently characterized with widely accessible instruments. This current FRAME-tag palette can be immediately used in yeast for basic biology and biotechnology. In addition, it can be possible to develop FRAME-tags for bacteria and mammalian cells, as −1 PRF naturally occurs in these cell types as well. It can also be possible to develop FRAME-tags for any other eukaryotic or prokaryotic cell, including fungal or plant cells. As basic biology tools, FRAME-tags can find use for investigating microbiome dynamics and pathogen engraftment, or for lineage tracing in developing organisms and tumors. Furthermore, as synthetic biology tools, FRAME-tags could be used in multicellular community engineering, distributed metabolic engineering, and biosensor arrays. With the aid of emerging genome-engineering technologies, automated cytometry, and time-lapse imaging, FRAME-tags and variants can find extensive use in the broader scientific community for high throughput, real-time, multicellular tracking.

Finally, FRAME-tags can be used to generate phenotypic cell libraries, for example cell libraries for new strains (FIG. 19). The mixed FRAME-tag DNA constructs can be co-transformed into the desired background strain. Barcoded transformants are directly pooled without tag identification, followed by transformation of pooled constructs or pre-screened libraries of constructs imparting a desired phenotype into the FRAME-tag strain mixture. Analysis is performed on individual colonies to first identify a set of strains with overrepresented FRAME-tag identities. The phenotypes of all members within this overrepresented set are identified and a subset of uniquely indexed phenotypes can be selected and used. Alternatively, pooled FRAME-tag DNA constructs can also be integrated into a preexisting pooled library of phenotypically variable strains followed by phenotype indexing as described.

The scalability limit of FP-based cell markers was overcome by harnessing ribosomal frameshifting to precisely encode non-overlapping FP expression ratios. With this approach, a total of 20 genetically encoded FRAME-tags were constructed using just two FPs and demonstrated the potential to scale to 100 tags with just three FPs. Importantly, this approach achieves scalability purely based on fluorescence signals. Three-FP FRAME-tags can be scaled further to potentially 1,000 tags using combinations of five currently available resolvable FP variants (5 choose 3 FP combinations×100 FRAME-tag designs). Furthermore, integrating FRAME-tags with spatial information, such as subcellular localization, could scale the palette exponentially.

To demonstrate their applicability, FRAME-tags were used to simultaneously track different sub-populations of cells in real-time and to perform multiplexed expression profiling in synthetic yeast communities. The scalability of the palette to potentially 100 FRAME-tags was established by simply including a third FP. This technology overcomes the FP multiplexing limit imposed by spectral overlap and enables straightforward tracking of complex cellular systems using widely available fluorescence techniques.

A diverse yeast community was also tracked in real time by exploiting rapid fluorescence data acquisition, which allowed the visualization of dynamic growth trajectories for all sub-populations simultaneously.

### Example 1: Generation of Frame-Tags

### Materials and Methods

### Materials

Polymerases, restriction enzymes and Gibson assembly mix were obtained from New England Biolabs (NEB) (Ipswich, Mass., USA). Media components were obtained from BD Bioscience (Franklin Lakes, N.J., USA) and Sigma Aldrich (St. Luis, Mo., USA). Oligonucleotides and synthetic DNA constructs were purchased from Integrated DNA Technologies (IDT) (Coralville, Iowa, USA). Plasmids were cloned and amplified in E. coli strain TG1 (Lucigen, Madison, Wis., USA) or C3040 (NEB). Human urine (Catalog No: IR100007P) was purchased from Innovative Research (Novi, Mich., USA). All other commercial chemical reagents were obtained from Sigma Aldrich. Bulk optical density and fluorescence measurements were made using an Infinite M200 plate reader (Tecan).

### Statistical Analysis

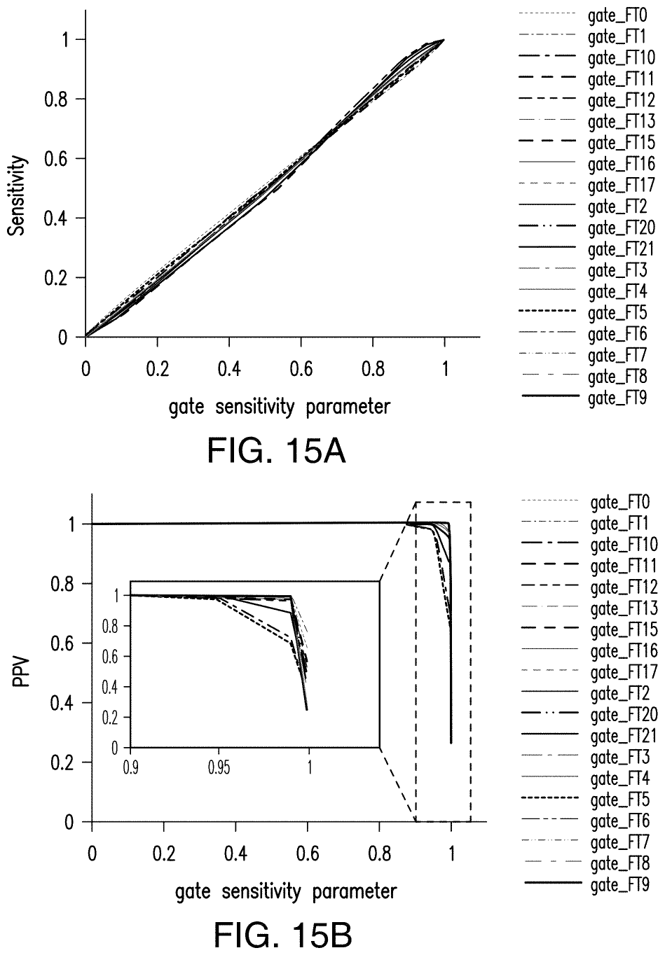

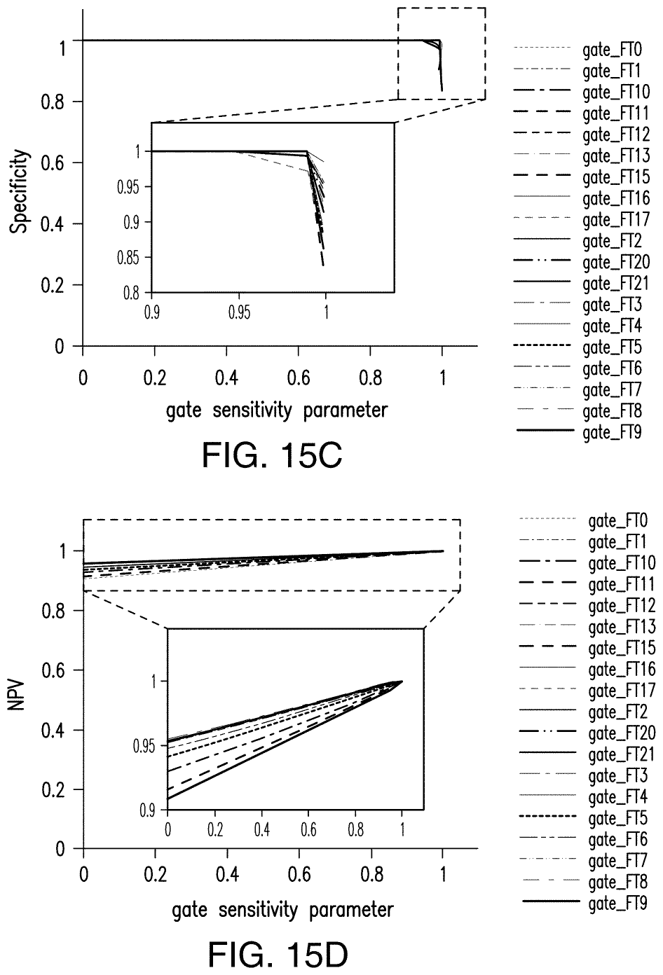

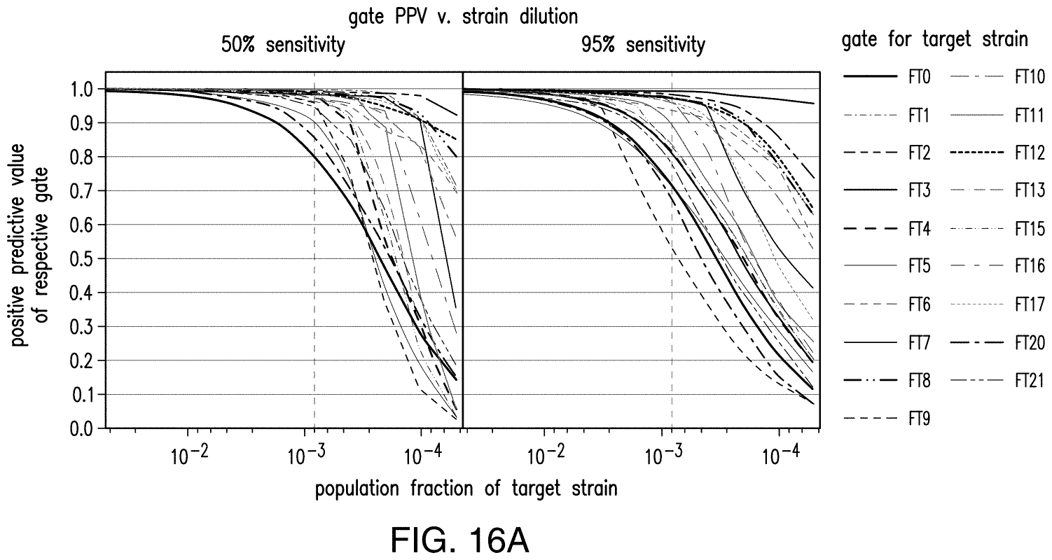

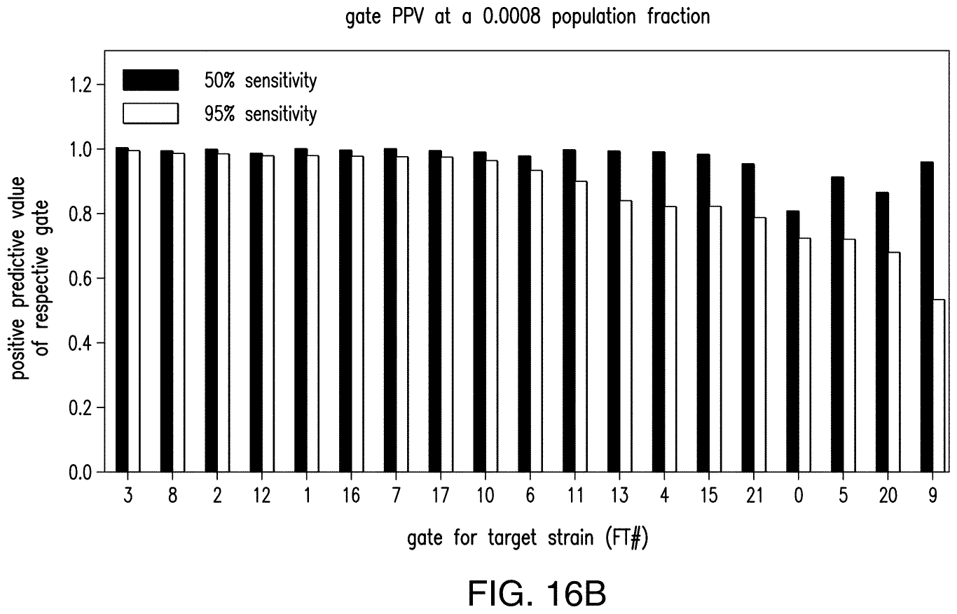

Overlap between mCherry fluorescence distributions (normalized to side scatter) for frameshift (fs) modules depicted in FIG. 1C and FIG. 8 was determined for all pairwise combinations. For each fs module's distribution, FS, an empirical cumulative density function CDF was generated using R. To estimate overlap between two distributions, FS, and FSⱼ, the point of intersection between CDFᵢ and (1−CDFⱼ) was approximated using uniroot (in R), and the degree of overlap was taken to be twice the value of the CDF at the intersection point. This was performed for all FS pairs to generate the matrix depicted in FIG. 8. Sensitivity, specificity, positive predictive value (PPV), and negative predictive value (NPV) for FRAME-tag flow cytometry gating algorithms were determined by evaluating data from FRAME-tags captured by flow cytometry individually (˜45,000 cells per FT) and combined into a dataset of all events. This allowed comparison of the automatic gating on this combined dataset relative to the known identities of the events. For a given FRAME-tag strain, FTₙ, the statistical parameters for its gate, Gₙ, were derived as follows. True positives (TP): events from individual analysis of FTₙ that fell within Gₙ; false negatives (FN): events from individual analysis of FTₙ that fell outside of Gₙ; true negatives (TN): combined events excluding FTₙ that fell outside of Gₙ; false positives (FP): combined events excluding FTₙ that fell within Gₙ. Further statistics were derived for each gate as follows. Sensitivity: TP/(TP+FN); specificity: TN/(TN+FP); PPV: TP/(TP+FP); NPV: TN/(TN+FN). These parameters were determined using several different gate sets generated at varying thresholds of sensitivity, i.e. the percentage of total cells captured by gates (see FIG. 15). Mock dilution studies of the gating pipeline were performed for each FRAME-tag strain by computationally constructing a new distribution using random subsampling from the original distribution, recombining with the other undiluted strain data and reapplying the automated gating pipeline, followed by the above statistical analyses (see FIG. 16).

### Designing a FRAME-Tag:

Frameshift Stimulatory Signal Structure Prediction.

FIG. 7B shows minimum free energy (mfe) RNA secondary structures were predicted computationally with pKiss using the ‘mfe’ function for non-pseudoknotted structures and the ‘local’ function for pseudoknotted structures (Janssen, S. & Giegerich, R., Bioinformatics 31, 423-425 (2015)). For each fs module, sequences of length 44, starting 8 nucleotides downstream of the slippery site, were evaluated for secondary structure. All calculations were performed at 37° C. with Turner 2004 default parameters in mode [P] to ignore kissing hairpins (K-type pseudoknots). Secondary structure diagrams are shown for the mfe of each fs module. Energies displayed correspond to that of the local pseudoknot or the first upstream hairpin in isolation within the mfe calculated at 37° C.

Plasmid Cloning and Genomic Integration in Yeast.

All yeast strains were derived from parental strains Fy251 [American Type Culture Collection (ATCC) 96098] or the two-hybrid strain MaV203 (Invitrogen) (Vidal, M. et al., Proc. Natl. Acad. Sci. 93, 10321-10326 (1996)). Yeast transformations were carried out using the lithium acetate method (Gietz, R. D. & Schiestl, R. H., Nat. Protoc. 2, 31-34 (2007)). All plasmids are derivatives of the pRS series of shuttle plasmids, cloned using standard molecular biology protocols, yeast gap repair and Gibson assembly. Endogenous yeast promoters were obtained by PCR from genomic DNA of strain Fy251. Genomic integration in yeast was done by homologous recombination of linearized DNA constructs with homology arms and a selectable marker. See Table 3 for a list of all strains used in this work. See Table 4 for a list of plasmids used in this work. See Table 5 for a list of primers used to clone endogenous promoters. See Table 6 for a list of DNA parts used to construct all FRAME-tags. FRAME-tag integration plasmids will be made available for distribution on Addgene.

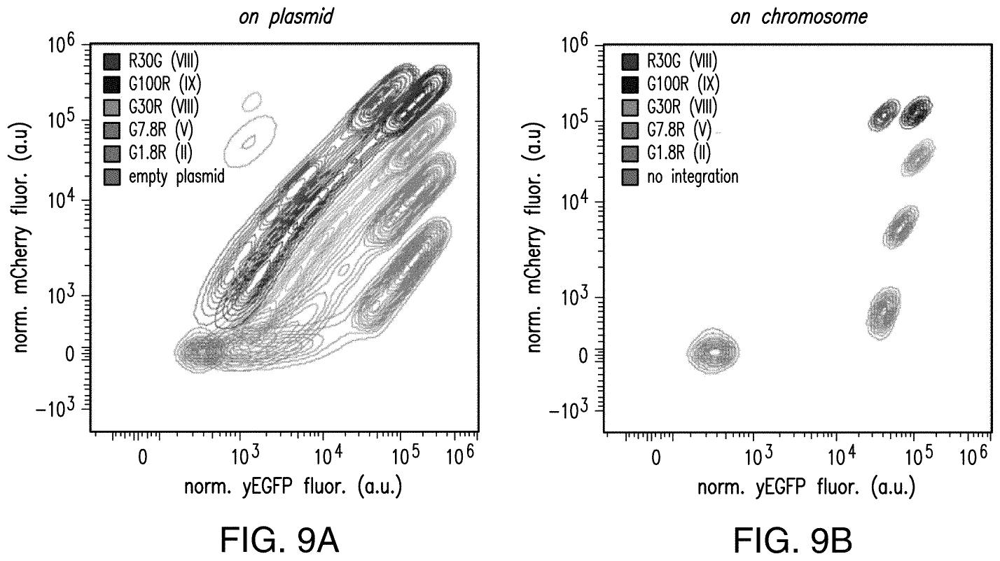

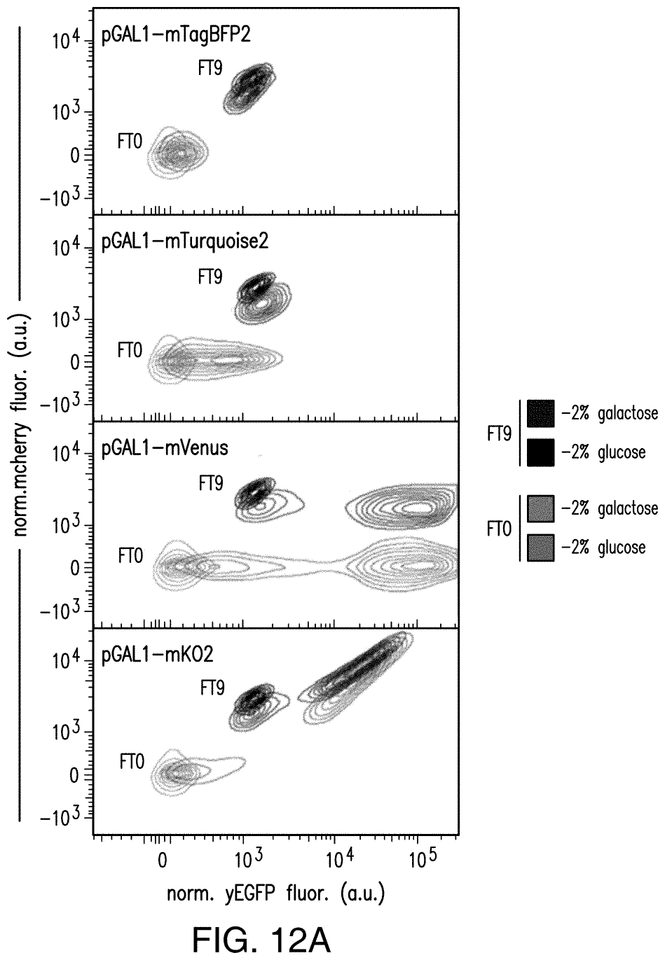

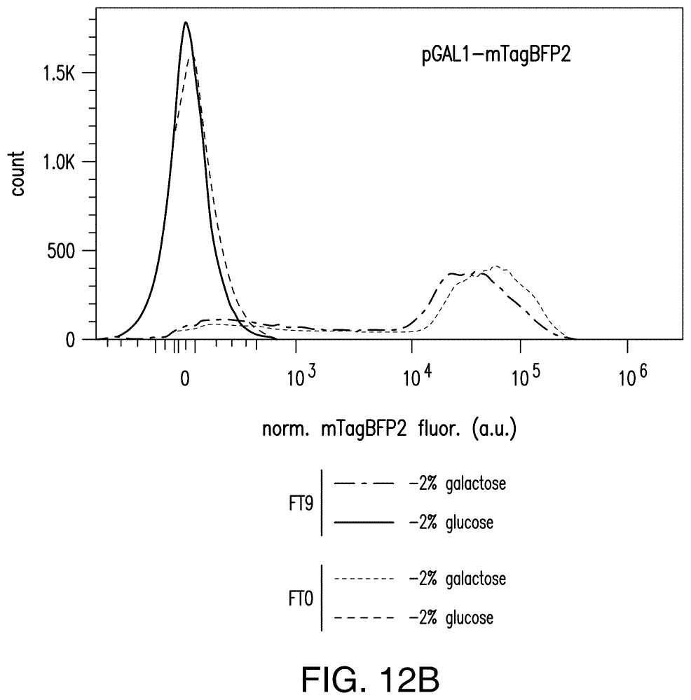

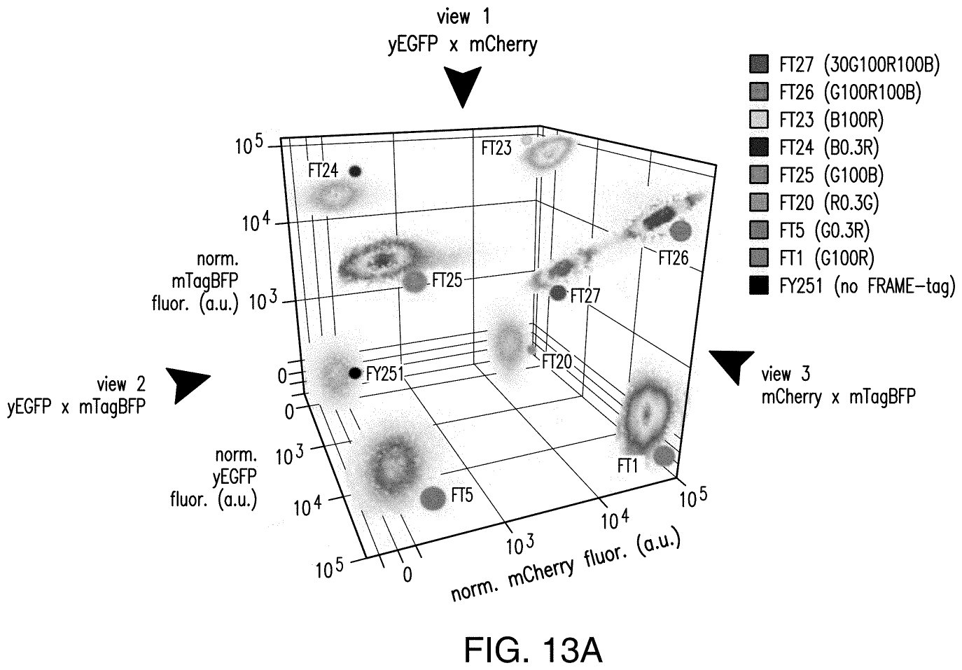

TABLE 3
Strains. Strains were generated in this study except where a source is noted.
Genotypes of FRAME-tagged strains given in fluorescent protein
(FP) nomenclature (e.g., G30R, see Table 2)

| Strain | Genotype | Comments |
|---|---|---|
| Parent strains | | |
| FY251 | MATa his3-Δ200 leu2-Δ1 trp1-Δ63 ura3-52 | ATCC 96098 |
| MaV203 | MATα leu2-3,112 trp1-901 his3Δ200 | |
| | ade2-101 cyh2R can1R gal4Δ gal80Δ | |
| | GAL1::lacZ HIS3_{UASGALI}::HIS3 @ LYS2 SPAL10::URA3 | |
| ƒs characterization and initial FRAME-tag (FT) strains | | |
| yFT1 | FY251 leu2Δ::CgLEU2-G100R | Also a final |
| | | dual-FP |
| | | red/green FT |
| | | strain |
| yFT2 | FY251 leu2Δ::CgLEU2-G30R | Also a final |
| | | dual-FP |
| | | red/green FT |
| | | strain |
| yG20R | FY251 leu2Δ: CgLEU2-G20R | |
| yFT3 | FY251 leu2Δ::CgLEU2-G9.4R | Also a final |
| | | dual-FP |
| | | red/green FT |
| | | strain |
| yG7.8R | FY251 leu2Δ::CgLEU2-G7.8R | |
| yFT4 | FY251 leu2Δ::CgLEU2-G4.2R | Also a final |
| | | dual-FP |
| | | red/green FT |
| | | strain |
| yG3.3R | FY251 leu2Δ::CgLEU2-G3.3R | |
| yG1.8R | FY251 leu2Δ::CgLEU2-G1.8R | |
| yFT5 | FY251 leu2Δ::CgLEU2-G0.3R | Also a final |
| | | dual-FP |
| | | red/green FT |
| | | strain |
| Plasmid characterization strains | | |
| yPFT1 | FY251 + pFT1 | Used in FIG. 9 |
| yPFT2 | FY251 + pFT1 | Used in FIG. 9 |
| yPFT6 | FY251 + pFT6 | Used in FIG. 9 |
| yPG7.8R | FY251 + pG7.8R | Used in FIG. 9 |
| yPG1.8R | FY251 + pG1.8R | Used in FIG. 9 |
| dual-FP red/green FT strains | | |
| yFT6 | FY251 leu2Δ::CgLEU2-R30G | |
| yFT7 | FY251 leu2Δ::CgLEU2-30G100R | |
| yFT8 | FY251 leu2Δ::CgLEU2-30G30R | |
| yFT9 | FY251 leu2Δ::CgLEU2-30G9.4R | |
| yFT10 | FY251 leu2Δ::CgLEU2-30G0.3R | |
| yFT11 | FY251 leu2Δ::CgLEU2-R9.4G | |
| yFT12 | FY251 leu2Δ::CgLEU2-30R30G | |
| yFT13 | FY251 leu2Δ::CgLEU2-9.4G100R | |
| yFT15 | FY251 leu2Δ::CgLEU2-9.4G0.3R | |
| yFT16 | FY251 leu2Δ::CgLEU2-R4.2G | |
| yFT17 | FY251 leu2Δ::CgLEU2-30R9.4G | |
| yFT19 | FY251 leu2Δ::CgLEU2-4.2G100R | |
| yFT20 | FY251 leu2Δ::CgLEU2-R0.3G | |
| yFT21 | FY251 leu2Δ::CgLEU2-30R0.3G | |
| yFT22 | FY251 leu2Δ::CgLEU2-9.4R0.3G | |
| dual-FP red/green/blue FT strains | | |
| yFT23 | FY251 leu2Δ::CgLEU2-B100R | |
| yFT24 | FY251 leu2Δ::CgLEU2-B0.3R | |
| yFT25 | FY251 leu2Δ::CgLEU2-G100B | |
| triple-FP red/green/blue FT strains | | |
| yFT26 | FY251 leu2Δ::CgLEU2-G1 00R1 00B | |
| yFT27 | FY251 leu2Δ::CgLEU2-30G100R100B | |
| Strains used to analyze compatibility third FPs | | |
| yFT0pBFP | FY251 + pGal-BFP | Used in FIG. 12 |
| yFT0pTurq | FY251 + pGal-Turq | Used in FIG. 12 |
| yFT0pVenus | FY251 + pGal-Venus | Used in FIG. 12 |
| yFT0pK02 | FY251 + pGal-KO2 | Used in FIG. 12 |
| yFT9pBFP | yFT9 + pGal-BFP | Used in FIG. 12 |
| yFT9pTurq | yFT9 + pGal-Turq | Used in FIG. 12 |
| yFT9pVenus | yFT9 + pGal-Venus | Used in FIG. 12 |
| yFT9pKO2 | yFT9 + pGal-KO2 | Used in FIG. 12 |
| Strains used for multiplex transcriptional profiling | | |
| yFT1p2 | yFT1 + pR2 | reporter: pTEF1 |
| yFT2p4 | yFT2 + pR4 | reporter: pSSA1 |
| yFT3p18 | yFT3 + pR18 | reporter: pERO1 |
| yFT4p16 | yFT4 + pR16 | reporter: pOLE1 |
| yFT5p19 | yFT5 + pR19 | reporter: pHXT1 |
| yFT6p15 | yFT6 + pR15 | reporter: pPMC1 |
| yFT7p13 | yFT7 + pR13 | reporter: pHIS4 |
| yFT8p5 | yFT8 + pR5 | reporter: pGDP1 |
| yFT9p10 | yFT9 + pR10 | reporter: pZRT1 |
| yFT10p3 | yFT10 + pR3 | reporter: pHSP12 |
| yFT11p20 | yFT11 + pR20 | reporter: pPRM5 |
| yFT12p9 | yFT12 + pR9 | reporter: pCUP1 |
| yFT13p22 | yFT13 + pGal-BFP | reporter: pGAL1 |
| yFT15p12 | yFT15 + pR12 | reporter: pARR3 |
| yFT16p7 | yFT16 + pR7 | reporter: pTRX2 |
| yFT17p14 | yFT17 + pR14 | reporter: pDAL5 |
| yFT19p1 | yFT19 + pR1 | reporter: pACT1 |
| yFT20p6 | yFT20 + pR6 | reporter: pFUS1 |
| yFT21p8 | yFT21 + pR8 | reporter: pRNR3 |
| yFT22p11 | yFT22 + pR11 | reporter: pFET3 |
| Strains used for community tracking | | |
| A | MaV203 leu2Δ::CgLEU2-R0.3G + pSynGal4-A | tag: FT20 |
| B | MaV203 leu2Δ::CgLEU2-G9.4R + pSynGal4-B | tag: FT3 |
| C | MaV203 leu2Δ::CgLEU2-30G30R + pSynGal4-C | tag: FT8 |
| D | MaV203 leu2Δ::CgLEU2-30G0.3R + pSynGal4-D | tag: FT10 |
| E | MaV203 leu2Δ::CgLEU2-9.4R0.3G + pSynGal4-E | tag: FT22 |
| F | MaV203 leu2Δ::CgLEU2-30G100R + pSynGal4-F | tag: FT7 |
| G | MaV203 leu2Δ::CgLEU2-G100R + pSynGal4-G | tag: FT1 |
| H | MaV203 leu2Δ::CgLEU2-R4.2G + pSynGal4-H | tag: FT16 |
| I | MaV203 leu2Δ::CgLEU2-30G9.4R + pSynGal4-I | tag: FT9 |

TABLE 4
Plasmids.

| Plasmid | Construct details | Comments |
|---|---|---|
| pRS416 | URA3, CEN6/ARS4, AmpR, ColE1 | ATCC 87521 |
| pRS424 | TRP1, 2μ, AmpR, ColE1 | ATCC 77105 |
| pGal-BFP | pRS416, pGAL1-mTagBFP2 | |
| pGal-Turq | pRS416, pGAL1-mTurquoise2 | |
| pGal-Venus | pRS416, pGAL1-mVenus | |
| pGal-KO2 | pRS416, pGAL1-mKO2 | |
| pG20R | pRS416, CgLEU2-G20R | framed with homology for LEU2 locus |
| pG7.8R | pRS416, CgLEU2-G7.8R | framed with homology for LEU2 locus |
| pG3.3R | pRS416, CgLEU2-G3.3R | framed with homology for LEU2 locus |
| pG1.8R | pRS416, CgLEU2-G1.8R | framed with homology for LEU2 locus |
| pFT1 | pRS416, CgLEU2-G100R | framed with homology for LEU2 locus |
| pFT2 | pRS416, CgLEU2-G30R | framed with homology for LEU2 locus |
| pFT3 | pRS416, CgLEU2-G9.4R | framed with homology for LEU2 locus |
| pFT4 | pRS416, CgLEU2-G4.2R | framed with homology for LEU2 locus |
| pFT5 | pRS416, CgLEU2-G0.3R | framed with homology for LEU2 locus |
| pFT6 | pRS416, CgLEU2-R30G | framed with homology for LEU2 locus |
| pFT7 | pRS416, CgLEU2-30G100R | framed with homology for LEU2 locus |
| pFT8 | pRS416, CgLEU2-30G30R | framed with homology for LEU2 locus |
| pFT9 | pRS416, CgLEU2-30G9.4R | framed with homology for LEU2 locus |
| pFT10 | pRS416, CgLEU2-30G0.3R | framed with homology for LEU2 locus |
| pFT11 | pRS416, CgLEU2-R9.4G | framed with homology for LEU2 locus |
| pFT12 | pRS416, CgLEU2-30R30G | framed with homology for LEU2 locus |
| pFT13 | pRS416, CgLEU2-9.4G100R | framed with homology for LEU2 locus |
| pFT15 | pRS416, CgLEU2-9.4G0.3R | framed with homology for LEU2 locus |
| pFT16 | pRS416, CgLEU2-R4.2G | framed with homology for LEU2 locus |
| pFT17 | pRS416, CgLEU2-30R9.4G | framed with homology for LEU2 locus |
| pFT19 | pRS416, CgLEU2-4.2G100R | framed with homology for LEU2 locus |
| pFT20 | pRS416, CgLEU2-R0.3G | framed with homology for LEU2 locus |
| pFT21 | pRS416, CgLEU2-30R0.3G | framed with homology for LEU2 locus |
| pFT22 | pRS416, CgLEU2-9.4R0.3G | framed with homology for LEU2 locus |
| pFT23 | pRS416, CgLEU2-B100R | framed with homology for LEU2 locus |
| pFT24 | pRS416, CgLEU2-B0.3R | framed with homology for LEU2 locus |
| pFT25 | pRS416, CgLEU2-G100B | framed with homology for LEU2 locus |
| pFT26 | pRS416, CgLEU2-G100R100B | framed with homology for LEU2 locus |
| pFT27 | pRS416, CgLEU2-30G100R100B | framed with homology for LEU2 locus |
| pR1 | pRS416, pACT1-mTagBFP2 | |
| pR2 | pRS416, pTEF1-mTagBFP2 | |
| pR3 | pRS416, pHSP12-mTagBFP2 | |
| pR4 | pRS416, pSSA1-mTagBFP2 | |
| pR5 | pRS416, pGPD1-mTagBFP2 | |
| pR6 | pRS416, pFUS1-mTagBFP2 | |
| pR7 | pRS416, pTRX2-mTagBFP2 | |
| pR8 | pRS416, pRNR3-mTagBFP2 | |
| pR9 | pRS416, pCUP1-mTagBFP2 | |
| pR10 | pRS416, pZRT1-mTagBFP2 | |
| pR11 | pRS416, pFET3-mTagBFP2 | |
| pR12 | pRS416, pARR3-mTagBFP2 | |
| pR13 | pRS416, pHIS4-mTagBFP2 | |
| pR14 | pRS416, pDAL5-mTagBFP2 | |
| pR15 | pRS416, pPMC1-mTagBFP2 | |
| pR16 | pRS416, pOLE1-mTagBFP2 | |
| pR18 | pRS416, pERO1-mTagBFP2 | |
| pR19 | pRS416, pHXT1-mTagBFP2 | |
| pR20 | pRS416, pPRM5-mTagBFP2 | |
| pSynGa14-A | pRS424, pADH-Ga14(BD)-fsA-Ga14(AD) | |
| pSynGa14-B | pRS424, pADH-Ga14(BD)-fsB-Ga14(AD) | |
| pSynGa14-C | pRS424, pADH-Ga14(BD)-fsC-Ga14(AD) | |
| pSynGa14-D | pRS424, pADH-Gal4(BD)-fsD-Gal4(AD) | |
| pSynGa14-E | pRS424, pADH-Gal4(BD)-fsE-Gal4(AD) | |
| pSynGa14-F | pRS424, pADH-Gal4(BD)-fsF-Gal4(AD) | |
| pSynGa14-G | pRS424, pADH-Gal4(BD)-fsG-Gal4(AD) | |
| pSynGa14-H | pRS424, pADH-Gal4(BD)-fsH-Gal4(AD) | |
| pSynGa14-I | pRS424, pADH-Gal4(BD)-fsI-Gal4(AD) | |

pFT plasmids contain the integrating constructs of the FRAME-tags and were not directly used in experiments except were noted. The integrating construct can be excised with SwaI, which cuts these pFT plasmids three times (twice flanking the FT construct and once in the backbone to simplify purification).

TABLE 5
Primers for promoter cloning.

| Primer | Sequence |
|---|---|
| pACT_for (MJ615) | ctcactaaagggaacaaaagctggagctct |
| | agtCCTTAAAAACATATGCCTCACCCT |
| pACT_rev (MJ616) | gcatattttctttaatcaattcagacattt |
| | tctagaCAGTAAATTTTCGATCTTGGGAAG |
| pTEF1_for (MJ617) | ctcactaaagggaacaaaagctggagctct |
| | agtATAGCTTCAAAATGTTTCTACTCCT |
| pTEF_rev (MJ618) | gcatattttctttaatcaattcagacattt |
| | tctagaTTAGATTGCTATGCTTTCTTTC |
| pHSP12_for (MJ619) | ctcactaaagggaacaaaagctggagctct |
| | agtAGTGAAAATCTCCGGGAGCG |
| pHSP12_rev (MJ620) | gcatattttctttaatcaattcagacattt |
| | tctagaTGAGTTGTTTGTTTGAGATTATCG |
| pSSA1_for (MJ621) | ctcactaaagggaacaaaagctggagctct |
| | agtGGCATTTTCGTTCTTGTGGA |
| pSSA1_rev (MJ622) | gcatattttctttaatcaattcagacattt |
| | tctagaATTTTTGTTTCTTGTAATACTTGA |
| pGPD1_for (MJ625) | ctcactaaagggaacaaaagctggagctct |
| | agtCTGGGGTTTGAGCAAGTCTA |
| pGPD1_rev (MJ626) | gcatattttctttaatcaattcagacattt |
| | tctagaTTATCAATATTTGTGTTTGTGGAG |
| pFUS1_for (MJ627) | ctcactaaagggaacaaaagctggagctct |
| | agtTGCCTCAATCCTTCTTTTGCTT |
| pFUS1_rev (MJ628) | gcatattttctttaatcaattcagacattt |
| | tctagaCTTGATGGCTTATATCCTGCTCT |
| pTRX2_for (MJ629) | ctcactaaagggaacaaaagctggagctct |
| | agtACTTTTACGGGTGGCAACG |
| pTRX2_rev (MJ630) | gcatattttctttaatcaattcagacattt |
| | tctagaTCGTAGACTCTCGTGTATGTGTGC |
| pRNR3_for (MJ631) | ctcactaaagggaacaaaagctggagctct |
| | agtGTAATAACAAGCAGGTGGGCG |
| pRNR3_rev (MJ632) | gcatattttctttaatcaattcagacattt |
| | tctagaTTATTGCTGCTGCTATTCTTGCTT |
| pCUP1_for (MJ635) | ctcactaaagggaacaaaagctggagctct |
| | agtTCACCACCCTTTATTTCAGGC |
| pCUP_rev (MJ636) | gcatattttctttaatcaattcagacattt |
| | tctagaTGTGATGATTGATTGATTGATTGT |
| pZRT1_for (MJ637) | ctcactaaagggaacaaaagctggagctct |
| | agtGGCAAGAGTATTTCAGACTTTCCT |
| pZRT1_rev (MJ638) | gcatattttctttaatcaattcagacattt |
| | tctagaTTTGTGCTGTTGTTTTATTGTCT |
| pFET3_for (MJ666) | ctcactaaagggaacaaaagctggagctct |
| | agtGATAATGCCTTGGCTTGCCT |
| pFET3_rev (MJ640) | gcatattttctttaatcaattcagacattt |
| | tctagaTACTCTTCCTTACACTGGGGTCC |
| pARR3_for (MJ641) | ctcactaaagggaacaaaagctggagctct |
| | agtCACGTGCAAAATCTTCTCTTCG |
| pARR3_rev (MJ642) | gcatattttctttaatcaattcagacattt |
| | tctagaCCTGATGATTTGTTGGTTGGGT |
| pHIS4_for (MJ643) | ctcactaaagggaacaaaagctggagctct |
| | agtAAACCCATGCACAGTGACTC |
| pHIS4_rev (MJ644) | gcatattttctttaatcaattcagacattt |
| | tctagaATTGTATTACTATTACACAGCGCA |
| pDAL5_for (MJ649) | ctcactaaagggaacaaaagctggagctct |
| | agtAGCGTTCTCATCAGTCACTTG |
| pDAL4_rev (MJ650) | gcatattttctttaatcaattcagacattt |
| | tctagaATCCTTGTTTTGTTGTTTTCTTCA |
| pPMC1_for (MJ651) | ctcactaaagggaacaaaagctggagctct |
| | agtGTTTTTACCCGGCAAAGAAGC |
| pPMC1_rev (MJ652) | gcatattttctttaatcaattcagacattt |
| | tctagaTATTTTTTTTGTTACGCACACAGT |
| pOLE1_for (MJ653) | ctcactaaagggaacaaaagctggagctct |
| | agtCATGTCCCGGGGTTAGCG |
| pOLE1_rev (MJ654) | gcatattttctttaatcaattcagacattt |
| | tctagaTTGTTGTAATGTTTTAGTGCTGT |
| pERO1_for (MJ623) | ctcactaaagggaacaaaagctggagctct |
| | agtAAAGAACACGGCGGTAAGAA |
| pERO1_rev (MJ624) | gcatattttctttaatcaattcagacattt |
| | tctagaTTTACCTGCACGTTACTGTGG |
| pHXT1_for (MJ633) | ctcactaaagggaacaaaagctggagctct |
| | agtTGCAAAAAGCTTCCGATCCT |
| pHXT1_rev (MJ634) | gcatattttctttaatcaattcagacattt |
| | tctagaCGTATATCAACTAGTTGACGATTA |
| pPRM5_for (MJ645) | ctcactaaagggaacaaaagctggagctct |
| | agtCTCACCCGGATCGTAGTCAC |
| pPRM5_rev (MJ646) | gcatattttctttaatcaattcagacattt |
| | tctagaTCTTGCGTTTTGAGTGTCAATTT |

TABLE 6
DNA sequences of FRAME-tag parts.

| Part | Sequence |
|---|---|
| pTDH3 | CAGTTCGAGTTTATCATTATCAATACTGCCATTTC |
| | AAAGAATACGTAAATAATTAATAGTAGTGATTTTC |
| | CTAACTTTATTTAGTCAAAAAATTAGCCTTTTAAT |
| | TCTGCTGTAACCCGTACATGCCCAAAATAGGGGGC |
| | GGGTTACACAGAATATATAACATCGTAGGTGTCTG |
| | GGTGAACAGTTTATTCCTGGCATCCACTAAATATA |
| | ATGGAGCCCGCTTTTTAAGCTGGCATCCAGAAAAA |
| | AAAAGAATCCCAGCACCAAAATATTGTTTTCTTCA |
| | CCAACCATCAGTTCATAGGTCCATTCTCTTAGCGC |
| | AACTACAGAGAACAGGGGCACAAACAGGCAAAAAA |
| | CGGGCACAACCTCAATGGAGTGATGCAACCTGCCT |
| | GGAGTAAATGATGACACAAGGCAATTGACCCACGC |
| | ATGTATCTATCTCATTTTCTTACACCTTCTATTAC |
| | CTTCTGCTCTCTCTGATTTGGAAAAAGCTGAAAAA |
| | AAAGGTTGAAACCAGTTCCCTGAAATTATTCCCCT |
| | ACTTGACTAATAAGTATATAAAGACGGTAGGTATT |
| | GATTGTAATTCTGTAAATCTATTTCTTAAACTTCT |
| | TAAATTCTACTTTTATAGTTAGTCTTTTTTTTAGT |
| | TTTAAAACACCAAGAACTTAGTTTCGAATAAACAC |
| | ACATAAACAAACAAA |
| L1 | ATGGGTTCAGGTGAACAATCA |
| L2 | GGCAGCGGCGACTACAAGGACGACGACGAC |
| L3 | GGTGCATCTGGATCAGGACAATCA |
| NheI | GCTAGC |
| AatII | GACGTC |
| SalI | GTCGAC |
| AfeI | AGCGCT |
| ClaI | ATCGAT |
| yEGFP | ATGTCTAAAGGTGAAGAATTATTCACTGGTGTTGT |
| | CCCAATTTTGGTTGAATTAGATGGTGATGTTAATG |
| | GTCACAAATTTTCTGTCTCCGGTGAAGGTGAAGGT |
| | GATGCTACTTACGGTAAATTGACCTTAAAATTTAT |
| | TTGTACTACTGGTAAATTGCCAGTTCCATGGCCAA |
| | CCTTAGTCACTACTTTCGGTTATGGTGTTCAATGT |
| | TTTGCTAGATACCCAGATCATATGAAACAACATGA |
| | CTTTTTCAAGTCTGCCATGCCAGAAGGTTATGTTC |
| | AAGAAAGAACTATTTTTTTCAAAGATGACGGTAAC |
| | TACAAGACCAGAGCTGAAGTCAAGTTTGAAGGTGA |
| | TACCTTAGTTAATAGAATCGAATTAAAAGGTATTG |
| | ATTTTAAAGAAGATGGTAACATTTTAGGTCACAAA |
| | TTGGAATACAACTATAACTCTCACAATGTTTACAT |
| | CATGGCTGACAAACAAAAGAATGGTATCAAAGTTA |
| | ACTTCAAAATTAGACACAACATTGAAGATGGTTCT |
| | GTTCAATTAGCTGACCATTATCAACAAAATACTCC |
| | AATTGGTGATGGTCCAGTCTTGTTACCAGACAACC |
| | ATTACTTATCCACTCAATCTGCCTTATCCAAAGAT |
| | CCAAACGAAAAGAGAGACCACATGGTCTTGTTAGA |
| | ATTTGTTACTGCTGCTGGTATTACCCATGGTATGG |
| | ATGAATTGTACAAA |
| mCherry | ATGGTTTCAAAAGGTGAAGAAGATAATATGGCTAT |
| | TATTAAAGAATTTATGAGATTTAAAGTTCATATGG |
| | AAGGTTCAGTTAATGGTCATGAATTTGAAATTGAA |
| | GGTGAAGGTGAAGGTAGACCATATGAAGGTACTCA |
| | AACTGCTAAATTGAAAGTTACTAAAGGTGGTCCAT |
| | TACCATTTGCTTGGGATATTTTGTCACCACAATTT |
| | ATGTATGGTTCAAAAGCTTATGTTAAACATCCAGC |
| | TGATATTCCAGATTATTTAAAATTGTCATTTCCAG |
| | AAGGTTTTAAATGGGAAAGAGTTATGAATTTTGAA |
| | GATGGTGGTGTTGTTACTGTTACTCAAGATTCATC |
| | ATTACAAGATGGTGAATTTATTTATAAAGTTAAAT |
| | TGAGAGGTACTAATTTTCCATCAGATGGTCCAGTT |
| | ATGCAAAAAAAAACTATGGGTTGGGAAGCTTCATC |
| | AGAAAGAATGTATCCAGAAGATGGTGCTTTAAAAG |
| | GTGAAATTAAACAAAGATTGAAATTAAAAGATGGT |
| | GGTCATTATGATGCTGAAGTTAAAACTACTTATAA |
| | AGCTAAAAAACCAGTTCAATTACCAGGTGCTTATA |
| | ATGTTAATATTAAATTGGATATTACTTCACATAAT |
| | GAAGATTATACTATTGTTGAACAATATGAAAGAGC |
| | TGAAGGTAGACATTCAACTGGTGGTATGGATGAAT |
| | TATATAAAagcgctGGTGCATCTGGATCAGGACAA |
| | TCAAAAGACTTTAAACTAGTTGACGCGGGTCTAGT |
| | CAACAACGCGTTAAACCCACTAGAAGGCGGTTCTA |
| | TGGGAATGTCTGGA |
| mTagBFP2 | ATGTCTGAATTGATTAAAGAAAATATGCATATGAA |
| | ATTGTACATGGAAGGTACTGTTGATAATCATCATT |
| | TCAAATGTACTTCAGAAGGTGAAGGTAAGCCATAC |
| | GAAGGTACTCAAACTATGAGAATTAAAGTTGTTGA |
| | AGGTGGTCCATTGCCATTCGCTTTTGATATTTTGG |
| | CTACTTCATTTTTATATGGTTCAAAGACTTTTATT |
| | AATCATACTCAAGGTATTCCAGATTTTTTCAAACA |
| | ATCATTCCCAGAAGGTTTCACTTGGGAAAGAGTTA |
| | CTACTTACGAAGATGGTGGTGTTTTAACTGCTACT |
| | CAAGATACTTCTTTGCAAGATGGTTGTTTGATTTA |
| | CAATGTTAAAATTAGAGGTGTTAATTTCACTTCAA |
| | ATGGTCCAGTTATGCAAAAAAAGACTTTGGGTTGG |
| | GAAGCTTTTACTGAAACTTTGTACCCAGCTGATGG |
| | TGGTTTGGAAGGTAGAAATGATATGGCTTTGAAGT |
| | TGGTTGGTGGTTCTCATTTGATTGCTAATGCTAAG |
| | ACTACTTATAGATCAAAGAAGCCAGCTAAGAATTT |
| | GAAGATGCCAGGTGTTTATTACGTTGATTATAGAT |
| | TGGAAAGAATTAAAGAAGCTAATAATGAAACTTAC |
| | GTTGAACAACATGAAGTTGCTGTTGCTAGATACTG |
| | TGATTTGCCATCAAAATTGGGTCATAAATTGAAT |
| tCaADH1 | TAAGCAAATAGCTAAATTATATACGAATTAATATT |
| | ATGATTAAGTGTTTACGTGAGTGCGATATTTTTAT |
| | TACTATCTTATACAGTTGTATATACTCTATAAAAT |
| | GAGTTGTCTATTAATTAACGCGATGAATGCTTTCT |
| | GGGTTTACCTCTCCAACAACTCTAGTTTACTTCTC |
| | AATACATTCAATTGTATTTGATTTGTCAATACTTC |
| | ATCATTAATCAATTCTATAGTTTTGTTTTTCTCGT |
| | TTATTTCCAAATTTAATGCATCAATTTTATTATTC |
| | AATTTGTCGTTGATTTTGGTTAATGATTTTATGGT |
| | TTGATCTCTGGCATTGATTGTTTGTGTTAGTTTTT |
| | CATTATTGATAattaaaTTATTTAAGTTAGTTATC |
| | AACTCGGTGTTTTCAAGTTTCAAGTTTTCAATTTC |
| | TTTAGAGTTTATTAGATTTGTCAAAGTTTCTGAAT |
| | TGCTTGATTGGTCCTGTAGAAGAGTATTTGTTGTT |
| | GTGGATAATTGATTCAATTTTTGAGACAATTGCTG |
| | GAAGGCGTTGAAATATCTAGCATCAATCTCATGGT |
| | TTTTTTCCCGAGAGTCTCGTAGATTCAATTGTTTT |
| | AATATATCTTGGGACCACTCTTGATTTGAACTCAT |
| | GGAAattaaaCTGGGTGTTGTGTTGTGGTGTAATG |
| | ATTGTACCCCCTTTGCTTATAATTGTGTGG |

These parts and fs modules were assembled into FRAME-tags as shown in Table 2.

FRAME-Tag DNA Constructs and Strains.

The yEGFP DNA sequence and mCherry DNA sequence were amplified from a previously constructed plasmid (Anzalone, A. V. et al., Nat. Methods 13, 453-458 (2016)). mTagBFP2 (Subach, O. M. et al., PLoS ONE 6, e28674 (2011)), mKO2 (Sakaue-Sawano, A. et al., Cell 132, 487-498 (2008)), mTurquoise2 (Goedhart, J. et al., Nat. Commun. 3, ncomms1738 (2012)), and mVenus (Nagai, T. et al., Nat. Biotechnol. 20, 87-90 (2002)), were obtained as synthetic DNA fragments that were codon optimized for S. cerevisiae with the IDT codon optimization tool (Integrated DNA Technologies) and the JCat Codon Adaptation tool (see Table 7) (Grote, A. et al., Nucleic Acids Res. 33, W526-W531 (2005)). A parent dual FP integration construct was derived from the pNH600 series of vectors (Zalatan, J. G. et al., Science 337, 1218-1222 (2012)), which harbor integration constructs containing a multiple cloning site, an ADH1 terminator from Candida albicans, selectable auxotrophic markers from Candida glabrata, and flanking 500 bp homology regions to the target locus (pNH605: LEU2). Full integration constructs were cloned into a pRS416 backbone to allow comparison of plasmid-borne and genome-integrated constructs from the same vector. −1 PRF sequences were amplified as a DNA library from the previously reported in vitro selection products and cloned into the parent dual FP integration vector by gap repair in the yeast strain Fy251 (Anzalone, A. V. et al., Nat. Methods 13, 453-458 (2016)). Individual clones were isolated by selection on -Ura and the ratios of yEGFP and mCherry fluorescence were assayed in 96-well plates using an Infinite M200 plate reader (Tecan). Plasmid variants representing a range of fluorescence ratios were sequenced, then linearized and integrated into the LEU2 locus of a fresh Fy251 strain. Transformants were selected on SC (glucose, -Leu) plates, and proper integration was confirmed by sequencing of locus-specific PCR amplified DNA. The expanded palette of dual-FP FRAME-tags was generated by individually constructing combinations of the 5 chosen frameshift modules at early and late positions, with two fluorescent proteins (Table 2). Vectors were linearized and integrated independently into Fy251, the resulting strains were pre-cultured as above and characterized by flow cytometry. Third fluorescent proteins were screened for compatibility by cloning into a galactose inducible construct (pRS416-Gal1). Sequence verified plasmids were transformed into green-red FRAME-tagged yeast strains and grown in SC (2% glucose, -Ura) or SC (2% galactose, 2% raffinose, -Ura) media. The contribution of the third fluorescent protein on both GFP and mCherry signals was evaluated by pre-culturing the strains as above and characterizing the fluorescence by flow cytometry (FIG. 12). mTagBFP2 was chosen as a compatible third fluorescent protein. A three FP set of FRAME-tags was generated by individually constructing the indicated combinations of FPs and frameshift modules (Table 2). The resulting constructs were integrated into strain Fy251, pre-cultured as above and characterized by flow cytometry.

TABLE 7
DNA sequences of
additional fluorescent proteins.

| Part | Sequence |
|---|---|
| mTurquoise2 | ATGTCTAAAGGTGAAGAATTATTCACTGGT |
| | GTTGTTCCAATTTTGGTTGAATTAGATGGT |
| | GATGTTAATGGTCATAAATTCTCTGTTTCT |
| | GGTGAAGGTGAAGGTGATGCTACTTATGGT |
| | AAGTTGACTTTGAAGTTTATTTGTACTACT |
| | GGTAAATTGCCAGTTCCATGGCCAACTTTG |
| | GTTACTACTTTGTCTTGGGGTGTTCAATGT |
| | TTTGCTAGATATCCAGATCATATGAAACAA |
| | CATGATTTCTTTAAATCTGCTATGCCAGAA |
| | GGTTACGTTCAAGAAAGAACTATTTTCTTT |
| | AAAGATGATGGTAATTACAAAACTAGAGCT |
| | GAAGTTAAATTCGAAGGTGATACTTTGGTT |
| | AATAGAATTGAATTGAAGGGTATTGATTTC |
| | AAAGAAGATGGTAATATTTTGGGTCATAAG |
| | TTGGAATACAATTACTTCTCTGATAATGTT |
| | TACATTACTGCTGATAAGCAAAAGAATGGT |
| | ATTAAGGCTAATTTCAAGATTAGACATAAT |
| | ATTGAAGATGGTGGTGTTCAATTAGCTGAT |
| | CATTATCAACAAAATACTCCAATTGGTGAT |
| | GGTCCAGTTTTGTTGCCAGATAATCATTAT |
| | TTGTCTACTCAATCTAAATTGTCTAAAGAT |
| | CCAAATGAAAAAAGAGATCATATGGTTTTG |
| | TTGGAATTTGTTACTGCTGCTGGTATTACT |
| | TTGGGTATGGATGAATTGTACAAA |
| mVenus | ATGTCTAAAGGTGAAGAATTATTCACTGGT |
| | GTTGTTCCAATTTTGGTTGAATTAGATGGT |
| | GATGTTAATGGTCATAAATTCTCTGTTTCT |
| | GGTGAAGGTGAAGGTGATGCTACTTATGGT |
| | AAGTTGACTTTGAAGTTGATTTGTACTACT |
| | GGTAAATTGCCAGTTCCATGGCCAACTTTG |
| | GTTACTACTTTGGGTTACGGTTTGCAATGT |
| | TTTGCTAGATATCCAGATCATATGAAACAA |
| | CATGATTTCTTTAAATCTGCTATGCCAGAA |
| | GGTTACGTTCAAGAAAGAACTATTTTCTTT |
| | AAAGATGATGGTAATTACAAAACTAGAGCT |
| | GAAGTTAAATTCGAAGGTGATACTTTGGTT |
| | AATAGAATTGAATTGAAGGGTATTGATTTC |
| | AAAGAAGATGGTAATATTTTGGGTCATAAG |
| | TTGGAATACAATTACAATTCTCATAATGTT |
| | TACATTACTGCTGATAAGCAAAAGAATGGT |
| | ATTAAGGCTAATTTCAAGATTAGACATAAT |
| | ATTGAAGATGGTGGTGTTCAATTAGCTGAT |
| | CATTATCAACAAAATACTCCAATTGGTGAT |
| | GGTCCAGTTTTGTTGCCAGATAATCATTAT |
| | TTGTCTTACCAATCTAAATTGTCTAAAGAT |
| | CCAAATGAAAAAAGAGATCATATGGTTTTG |
| | TTGGAATTTGTTACTGCTGCTGGTATTACT |
| | TTGGGTATGGATGAATTGTACAAA |
| mKO2 | ATGTCTGTTATTAAGCCAGAAATGAAAATG |
| | AGATATTATATGGATGGTTCTGTTAATGGT |
| | CATGAATTTACTATTGAAGGTGAAGGTACT |
| | GGTAGACCATATGAAGGTCATCAAGAAATG |
| | ACTTTGAGAGTTACTATGGCTGAAGGTGGT |
| | CCAATGCCATTCGCTTTTGATTTGGTTTCT |
| | CATGTTTTCTGTTACGGTCATAGAGTTTTC |
| | ACTAAGTACCCAGAAGAAATTCCTGATTAT |
| | TTTAAGCAAGCTTTTCCAGAAGGTTTATCT |
| | TGGGAAAGATCATTGGAATTTGAAGATGGT |
| | GGTTCTGCTTCTGTTTCTGCTCATATTTCT |
| | TTGAGAGGTAATACTTTCTATCATAAGTCT |
| | AAATTTACTGGTGTTAATTTTCCAGCTGAT |
| | GGTCCAATTATGCAAAATCAATCTGTTGAT |
| | TGGGAACCATCTACTGAAAAAATTACTGCT |
| | TCTGATGGTGTTTTAAAAGGTGATGTTACT |
| | ATGTATTTGAAATTGGAAGGTGGTGGTAAT |
| | CATAAATGTCAAATGAAAACTACTTATAAA |
| | GCTGCTAAAGAAATTTTGGAAATGCCAGGT |
| | GATCATTATATTGGACATAGATTGGTTAGA |
| | AAGACTGAAGGTAATATTACTGAACAAGTT |
| | GAAGATGCTGTTGCTCATTCT |

These coding DNA sequences were codon-optimized for expression in yeast.

Selected fs modules were inserted between yEGFP and mCherry, then chromosomally integrated into yeast; mCherry fluorescence was evaluated by flow cytometry and normalized by side scatter; values above distributions indicate frameshift efficiency determined by comparison of mCherry/yEGFP ratios with the 100% yEGFP-mCherry fusion (see FIG. 8).

To begin constructing the FRAME-tag palette, several fs modules were screened in a yeast dual-FP reporter (FIG. 8 and Table 1). Frameshift (fs) modules were characterized in a dual-fluorescent protein reporter construct (FIG. 8A). Then, nine distinct frameshift modules were characterized with the dual-fluorescent protein reporter, chromosomally integrated in yeast, and analyzed by flow cytometry (FIG. 8B). Modules that were selected for FRAME-tags are shaded in purple. mCherry fluorescence was normalized by side scatter. Each frameshift module resulted in a characteristic frameshift efficiency that leads to a unique stoichiometry between mCherry and yEGFP (FIG. 8C). Motifs selected for FRAME-tags are bolded. Frameshifting efficiency was defined by the ratio of the mCherry to yEGFP fluorescence, by first subtracting the baseline fluorescence of a non-fluorescent strain (FY251) and defining fs-100 (non-frameshift fusion protein) as 100% efficiency. The final set offs modules was selected by choosing a set with mutually minimal overlaps in their empirical cumulative density functions noted by the squares outlined in black (FIG. 8D). Frameshift efficiency calculations and raw heatmap data are included in Supplementary Data 3.

A set of five modules were identified with highly resolved mCherry (a fluorescent protein) fluorescence distributions, which displayed less than 1% overlap between adjacent populations (FIG. 1C). In addition, flow cytometry revealed that these fs modules produced even tighter distributions of the mCherry:yEGFP fluorescence ratios (FIG. 9). FIG. 9 compares FRAME-tags using flow cytometry that are (FIG. 9A) expressed from a CEN plasmid (low copy) or (FIG. 9B) expressed from the chromosome. Individual samples overlaid and identified by color. Fluorescence was normalized by side scatter; contours represent 5% quantiles. As both fluorescent proteins are translated from a single mRNA, the relative ratio of FPs is robust to intrinsic biological noise that originates from variability in transcription, translation initiation, and mRNA stability. Moreover, it was found that absolute fluorescence could be made robust to extrinsic biological noise by single copy chromosomal integration (FIG. 9).

FIG. 2 shows two-color FRAME-tags were combinatorially constructed from two FPs (yEGFP and mCherry) and the five fs modules shown in FIG. 1C. FIG. 2A shows a graph of FRAME-tag constructs that were chromosomally integrated in yeast, analyzed by flow cytometry and normalized by side scatter; data displayed as a scatter plot from twenty FRAME-tag strains grown in mixed culture, colored by event density. The FRAME-tag design was expanded to three FPs (yEGFP, mCherry and mTagBFP2) to generate both two-FP and three-FP tags (including bracketed module) (FIG. 2B). Three-color FRAME-tags were analyzed as in FIG. 2A and visualized in a three-dimensional scatter plot colored by event density. Some FRAME-tags (FT1, FT5, FT20) are labeled for reference between FIG. 2A and b.

### Results

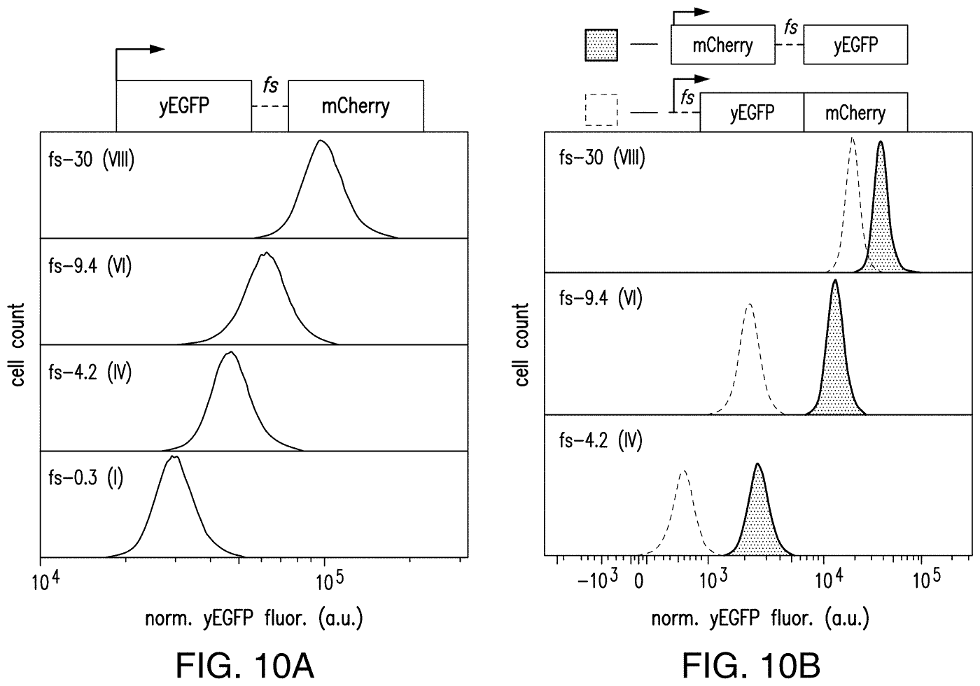

A two-FP FRAME-tag construct was designed wherein each FP is paired with a single upstream fs module (FIG. 2A and Table 2). In this design, translation of a given FP requires successful frameshifting at all upstream fs modules. As a result, the expected FP yield is roughly the product of preceding frameshifting efficiencies, with predictable deviations that depend on the location and strength of the RNA signals (FIG. 10). For example, FIG. 10A shows that that frameshift modules (fs) influence the expression of the upstream open reading frame. Efficiency offs is calculated as in FIG. 8. FIG. 10 compares yEGFP fluorescence controlled by late (purple) or early (black)fs modules. The effective yEGFP expression from the same fs module depends on its position within the construct (i.e. late or early). The same fs modules were used as in FIG. 10A, identified by their efficiency.

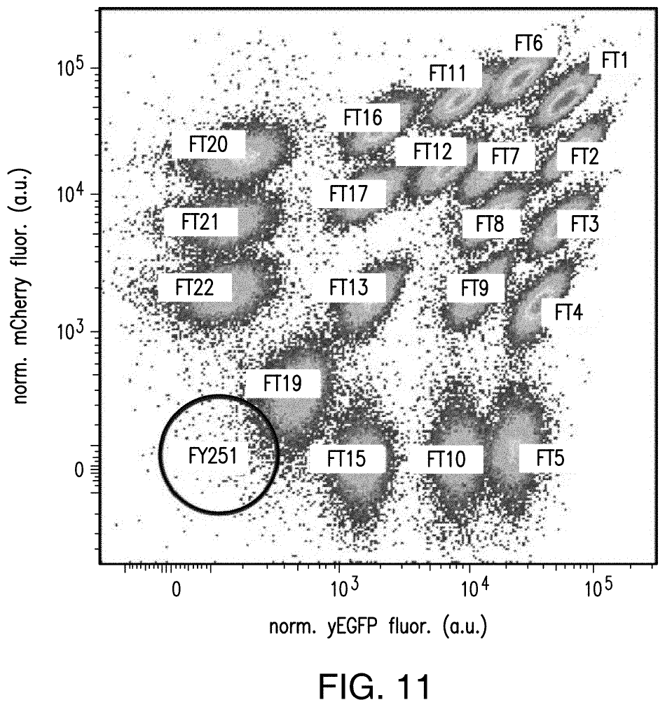

The consistency of these fs modules allowed the generation of a palette of 20 unique FRAME-tags in yeast that were well resolved by flow cytometry and microscopy using only two FPs, though more can be used (FIG. 2A and FIG. 11). FIG. 11 shows the visual index of FRAME-tags. The corresponding strain identity of each population was validated with single strain experiments and the FT# names used throughout are labeled in red. The approximate location of a non-fluorescent strain (FY251) is noted by the black outlined circle.

It was also found that this initial FRAME-tag series could be expanded to a third color dimension by the introduction of one additional FP variant. After screening several FPs, it was determined that the blue mTagBFP2 (Subach, O. M. et al., PLoS ONE 6, e28674 (2011)) is compatible with yEGFP and mCherry fluorescence channels (FIG. 12). FIG. 12 shows the identification of a compatible third fluorescent protein. yEGFP and mCherry flow cytometry 10% contour plots of FRAME-tagged strains expressing a galactose-inducible reporter are shown in FIG. 12A. Strains tagged with either FT9 (blue, black) or no tag (FT0, purple, grey) were transformed with a plasmid carrying a third fluorescent protein (mTagBFP2, mTurquoise2, mVenus or mKO2) under the control of a galactose inducible promoter (pGAL1). These strains were grown in either standard synthetic dropout medium (2% glucose, black and grey) or 2% galactose medium (blue, purple). The yEGFP and mCherry signal of the tags was not affected by expression of mTagBFP2 construct (colors v greys). FIG. 12B is a histogram of the of the mTagBFP2 signal from the pGAL1-mTagBFP2 strains in FIG. 12A. Baseline and induced of expression of mTagBFP2 is not influenced by the presence of a yEGFP/mCherry signal (FT9 v. FT0).

To establish scalability, three additional two-FP FRAME-tags were generated containing mTagBFP2, and a FRAME-tag construct that expresses all three FPs regulated was designated by upstream fs modules. The latter design was validated by the construction of two additional FRAME-tags (FIG. 2B and FIG. 13). FIG. 13 depicts the fluorescence of the additional FRAME-tags in RGB space. By including just one additional FP (mTagBFP2) the full combinatorial RGB space becomes available, and up to 100 FRAME-tags could be accessed (discussed above). FIG. 13A shows three additional dual-FP FRAME-tags that use the blue dimension (FT23, FT24, FT25), and two triple-FP FRAME-tags that use all three color dimensions (FT26, FT27). Scatterplot of a mixed sample including all noted strains, plotted in 3D using Plotly (https://plot.ly). Events shaded by density (low=dark blue, high=dark red) and populations identified from single strain experiments labeled with corresponding strain identity and a colored circle according to the legend. Legend includes both FT and FP nomenclature from Table 2. Transparency added to some events to clarify perspective. FIG. 13B shows single strain experiments overlaid and identified by color as in FIG. 13A. Views correspond to those noted in FIG. 13A. Fluorescence normalized by side scatter.

Based on these results demonstrating consistently narrow distributions of both two-color and three-color FRAME-tag populations, it is estimated that up to 100 unique FRAME-tags can fit within this three-color FP space (as discussed above).

TABLE 1
Sequences of frameshift modules

| Motif | Sequence |
|---|---|
| fs-0.3 (I) | AA-GACTTTAAACTAGTTGACGCGCAACTAAT |
| | TCAGGCCGCGTTAAACGTTCTAGAAGGCGGTT |
| | CTATGGGAATGTCTGGA |
| fs-1.8 (II) | AA-GACTTTAAACTAGTTGACGCGGGTCTAGT |
| | CAACAACGCGTTAAACCCACTAGAAGGCGGTT |
| | CTATGGGAATGTCTGGA |
| fs-3.3 (III) | AA-GACTTTAAACTAGTTGACGCGGCTCTAGT |
| | CGCAGTCGCGTTAAACAAGCTAGAAGGCGGTT |
| | CTATGGGAATGTCTGGA |
| fs-4.2 (IV) | AA-GACTTTAAACTAGTTGACGCGTCGCTAAC |
| | ACGTGGCGCGTTAAACCATCTAGAAGGCGGTT |
| | CTATGGGAATGTCTGGA |
| fs-7.8 (V) | AA-GACTTTAAACTAGTTGACGCGTCACTAGT |
| | AGCGGGCGCGTTAAACAAACTAGAAGGCGGTT |
| | CTATGGGAATGTCTGGA |
| fs-9.4 (VI) | AA-GACTTTAAACTAGTTGACGCGGATCTAGC |
| | TTGTAACGCGTTAAACCAGCTAGAAGGCGGTT |
| | CTATGGGAATGTCTGGA |
| fs-20 (VII) | AA-GACTTTAAACTAGTTGACGCGAGTCTAGG |
| | GGATAACGCGTTAAACTTCCTAGAAGGCGGTT |
| | CTATGGGAATGTCTGGA |
| fs-30 (VIII) | AA-GACTTTAAACTAGTTGACGCGTCGCTACC |
| | GCCCGGCGCGTTAAACACACTAGAAGGCGGTT |
| | CTATGGGAATGTCTGGA |
| fs-100 (IX) | AAAGACTTTAAACTAGTTGACGCGGGTCTAGT |
| | CAACAACGCGTTAAACCCACTAGAAGGCGGTT |
| | CTATGGGAATGTCTGGA |

TABLE 2
FRAME-tag constructs

| FT # | FP nom. | Construct assembly |
|---|---|---|
| FT1 | G100R | pTDH3•yEGFP•[NheI]•L2•fs-100•[AatIl]•mCherry•tCaADH1 |
| FT2 | G30R | pTDH3•yEGFP•[NheI]•L2•fs-30•[AatIl]•mCherry•tCaADH1 |
| FT3 | G9.4R | pTDH3•yEGFP•[NheI]•L2•fs-9.4•[AatIl]•mCherry•tCaADH1 |
| FT4 | G4.2R | pTDH3•yEGFP•[NheI]•L2•fs-4.2•[AatIl]•mCherry•tCaADH1 |
| FT5 | G0.3R | pTDH3•yEGFP•[NheI]•L2•fs-0.3•[AatIl]•mCherry•tCaADH1 |
| FT6 | R30G | pTDH3•mCherry•[NheI]•L2•fs-30•[AatIl]•yEGFP•tCaADH1 |
| FT7 | 30G100R | pTDH3•L1•fs-30•[SalI]•yEGFP•[NheI]•L2•fs-100•[AatIl]•mCherry•tCaADH1 |
| FT8 | 30G30R | pTDH3•L1•fs-30•[SalI]•yEGFP•[NheI]•L2•fs-30•[AatIl]•mCherry•tCaADH1 |
| FT9 | 30G9.4R | pTDH3•L1•fs-30•[SalI]•yEGFP•[NheI]•L2•fs-9.4•[AatIl]•mCherry•tCaADH1 |
| FT10 | 30G0.3R | pTDH3•L1•fs-30•[SalI]•yEGFP•[NheI]•L2•fs-0.3•[AatIl]•mCherry•tCaADH1 |
| FT11 | R9.4G | pTDH3•mCherry•[NheI]•L2•fs-9.4•[AatIl]•yEGFP•tCaADH1 |
| FT12 | 30R30G | pTDH3•L1•fs-30•[SalI]•mCherry•[NheI]•L2•fs-30•[AatIl]•yEGFP•tCaADH1 |
| FT13 | 9.4G100R | pTDH3•L1•fs-9.4•[SalI]•yEGFP•[NheI]•L2•fs-100•[AatIl]•mCherry•tCaADH1 |
| FT15 | 9.4G0.3R | pTDH3•L1•fs-9.4•[SalI]•yEGFP•[NheI]•L2•fs-0.3•[AatIl]•mCherry•tCaADH1 |
| FT16 | R4.2G | pTDH3•mCherry•[NheI]•L2•fs-4.2•[AatIl]•yEGFP•tCaADH1 |
| FT17 | 30R9.4G | pTDH3•L1•fs-30•[SalI]•mCherry•[NheI]•L2•fs-9.4•[AatIl]•yEGFP•tCaADH1 |
| FT19 | 4.2G100R | pTDH3•L1•fs-4.2•[SalI]•yEGFP•[NheI]•L2•fs-100•[AatIl]•mCherry•tCaADH1 |
| FT20 | R0.3G | pTDH3•mCherry•[NheI]•L2•fs-0.3•[AatIl]•yEGFP•tCaADH1 |
| FT21 | 30R0.3G | pTDH3•L1•fs-30•[SalI]•mCherry•[NheI]•L2•fs-0.3•[AatIl]•yEGFP•tCaADH1 |
| FT22 | 9.4R0.3G | pTDH3•L1•fs-9.4•[SalI]•mCherry•[NheI]•L2•fs-0.3•[AatIl]•yEGFP•tCaADH1 |
| FT23 | B100R | pTDH3•mTagBFP2•[NheI]•L2•fs-100•[AatIl]•mCherry•tCaADH1 |
| FT24 | B0.3R | pTDH3•mTagBFP2•[NheI]•L2•fs-0.3•[AatIl]•mCherry•tCaADH1 |
| FT25 | G100B | pTDH3•yEGFP•[NheI]•L2•fs-100•[AatIl]•mTagBFP2•tCaADH1 |
| FT26 | G100R100B | pTDH3•yEGFP•[NheI]•L2•fs-100•[AatIl]• |
| | | mCherry•[AfeI]•L3•fs-100•[ClaI]•mTagBFP2•tCaADH1 |
| FT27 | 30G100R100B | pTDH3•L1•fs-30•[SalI]•yEGFP•[NheI]•L2•fs-100• |
| | | [AatIl]•mCherry•[AfeI]•L3•fs-100•[ClaI]•mTagBFP2•tCaADH1 |

FTs are provided in FP/ƒs nomenclature where G = yEGFP, R = mCherry, B = mTagBFP2 and #-ƒs-# (e.g., G30R) and full construct sequence layout. See DNA sequences of each in Table 1 and Table 6. Restriction sites facilitate replacement of FP and ƒs modules.

### Example 2: Identifying Populations Using Automated Methods

To streamline data analysis for FRAME-tag applications, a method based on the R package openCyto that exploits the characteristically narrow FRAME-tag population distributions (FIG. 14) is presented. FIG. 14 details the method. FIG. 14A shows an example gating hierarchy dynamically generated with the method and implemented with openCyto. This approach primarily uses fast 1D bisecting gates along four derived parameters: normalized yEGFP (green), normalized mCherry (red), fluorescence ratio (blue) and total fluorescence (pink). The bisection does not necessarily divide the graph into equal halves. The final FT populations are gated using a fast 2D single cluster gate (black) that allows specifying the desired sensitivity by choice of the percentile captured. Parent populations illustrated in FIG. 14 parts B-G are in black outlined ovals of FIG. 14A and final FT populations in grey ovals. FIG. 14 parts B-G detail the steps of isolation population FT2. Gate colors as in FIG. 14A. In each panel, left insets show the 1D histogram bisecting gates, and the scatter plots show the resulting gated subpopulation (orange highlight and label). Parent population events highlighted in bright colors (names underlined) and overlaid on all original events in faded colors. Right plot in panel FIG. 14G, shows all resulting FT gates derived in the same manner illustrated for the FT2 gate. Populations corresponding to FT2, FT5 and FT20 labeled for reference. Normalized fluorescence values plotted using arbitrary logical units. Fluorescence ratio and total fluorescence values plotted using arbitrary log units. The raw flow cytometry data of this representative example is included in Supplementary Data 1, pre-gated to remove extreme scatter and fluorescent values (nonMaxF) and including the derived parameters (normalized GFP, normalized RFP, fluorescence ratio, fluorescence total). The raw data from a representative microscopy experiment is also included in Supplementary Data 2, including sectioned cell areas and ferret in pixel units (1 px=0.33 μm).

The statistical analysis of the method revealed gate positive predictive values (PPV) ranging from 0.99 to >0.9999 (mean gate PPV of 0.9987) at a cell detection sensitivity level of 90% (FIG. 15). FIG. 15 depicts the statistical analysis of the automated gating pipeline. FIG. 15A) sensitivity, b) positive predictive value (PPV), c) specificity, and d) negative predictive value (NPV) were calculated from gating data produced at various gate sensitivity parameters (0.001, 0.01, 0.1, 0.5, 0.8, 0.9, 0.95, 0.99, and 0.999) corresponding to the percentile of cells captured in the final FT gate relative to its immediate parent population (e.g., percentile of events in the FT2 gate relative to events in the FP2p gate, see FIG. 14G, black gates). Statistics are shown plotted vs. this gating sensitivity parameter. For details on statistical calculations, see the Statistical analysis section above. See Supplementary Data 4 for the corresponding numeric values.

Extrapolation of these data predicts that a large majority of FRAME-tagged strains could be detected with a PPV of greater than 0.9 for dilutions down to as low as 1 in >10³ while maintaining a cell detection rate of 50% (FIG. 16). FIG. 16 shows the detection limit of dilute populations for the method described above. In FIG. 16A, the flow cytometry experiments for all single strains were run independently and programmatically combined into a mock mixed dataset. This allowed generation of dilution series for each strain within this mixed dataset through subsampling of the events known to belong to that strain, while maintaining all other events unchanged. For each new dilution dataset, the automated gating pipeline was run and the positive predictive values (PPV) of the resulting gates were calculated. This was repeated at a gating sensitivity parameter of 50% and 95% which corresponds directly to real statistical sensitivity (see FIG. 15A). FIG. 16B shows data from FIG. 16A (at dashed lines) plotted at a single dilution of 0.0008 population fraction of the target strain to be detected, shows that a majority of strains can be correctly gated by the software at this dilution to give a PPV above 0.9 with a sensitivity of 50%. See Supplementary Data 5 for the corresponding numeric values.

### Example 3: Identifying Frame-Tagged Populations Using Fluorescence Microscopy

### Materials and Methods

### Flow Cytometry

Characterization of the FY251-based FRAME-tag strains including the multiplex transcriptional profiling was performed on a LSR II (Becton Dickinson) using the following laser/filter sets: 488/525 for yEGFP; 594/620 for mCherry; 405/450 for mTagBFP2. For standard analysis, FRAME-tagged strains were individually pre-cultured overnight in 96-well plates in standard synthetic dropout media (2% glucose) at 30° C. and 800 RPM, then inoculated at an OD₆₀₀ of 0.1 into fresh medium as individual strains or mixtures and grown for a further 10 hours. Cells were harvested by centrifugation, kept as pellets on ice, and analyzed within two hours. High throughput characterization of the MaV203-based FRAME-tag strains, including construction and real-time tracking of the yeast community, was performed on an LSR Fortessa (Becton Dickinson) using the following laser/filter sets: 488/530 for yEGFP; 561/610 for mCherry. Individual strains or mixtures of strains were cultured as described in flat-bottom 96-well plates and directly analyzed on the flow cytometer using a High Throughput Sampler (HTS, Becton Dickinson) in standard mode. All fluorescence signals were normalized by side scatter as a proxy for cell size and reported as arbitrary normalized fluorescence units scaled by 100,000 (Zuleta, I. A. et al., Nat. Methods 11, 443-448 (2014)). Automated gating and data analysis was carried out using the method based on the R package openCyto (see the method described above).

### Fluorescence Microscopy

FRAME-tagged strains were grown as described above. The mixtures of FRAME-tag strains were imaged on standard microscope slides with coverslips using a Ti-E microscope with Perfect Focus System (Nikon), a CFI Plan Apochromat Lambda 20X objective and a Zyla sCMOS camera (Andor). Excitation/emission (nm) sets used were: 470/525 for yEGFP; 555/620 for mCherry. For each experiment, 10-15 fields were automatically collected representing 8,000 to 12,000 cells. Bright field and fluorescence images were sectioned with FIJI using a custom script to extract average fluorescence values of individual cells (Schindelin, J. et al., Nat. Methods 9, 676 (2012)). The resulting data was input into the automated FRAME-tag gating and analysis software in R (see above) to index each cell with its respective FRAME-tag identity. These indices were used in FIJI to colorize the original bright filed images using the ROI Color Coder (Ferreira, T. et al., Zenodo (2015). doi:10.5281/zenodo.28838).

### Results

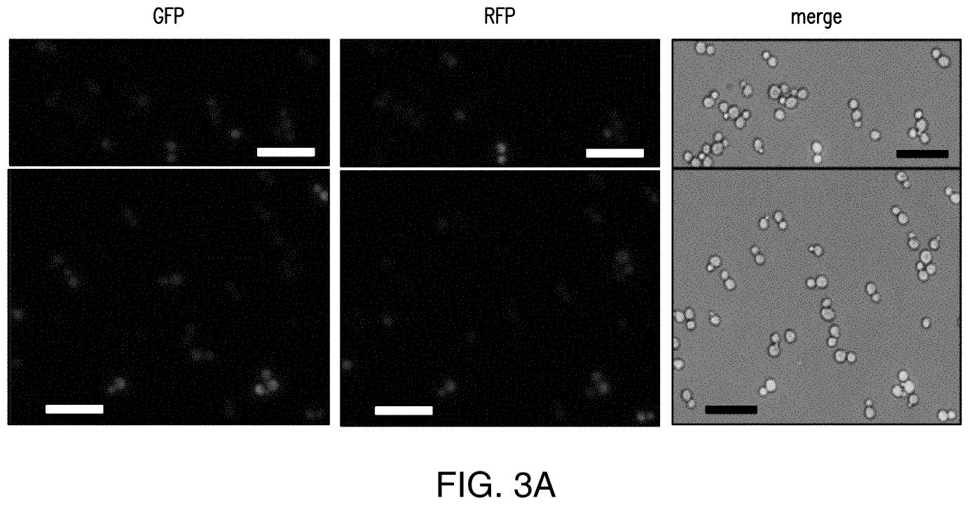

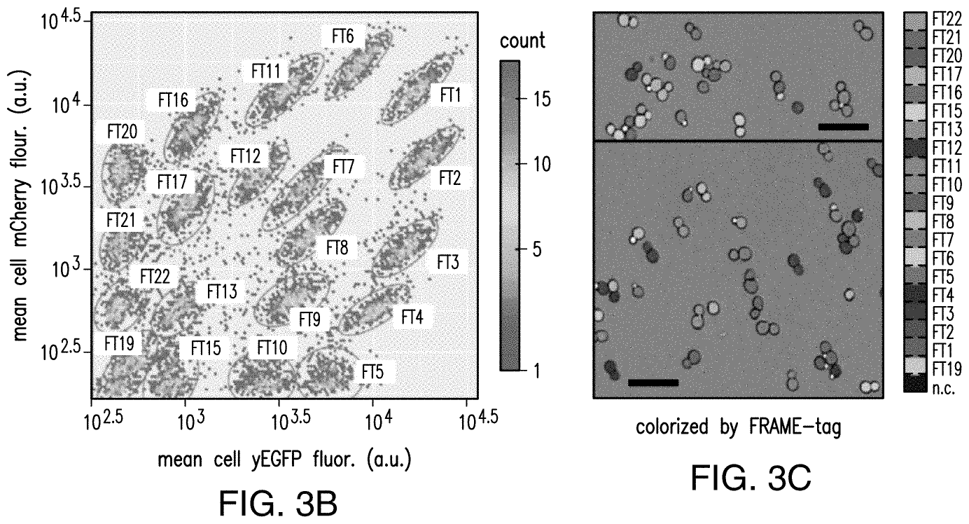

Fluorescence microscopy can be used to characterize FRAME-tags. Using two-color microscopy, samples of 10⁴ cells were imaged and fluorescence intensity values determined for each cell by image segmentation (FIG. 3A). The images showed a mixture of live yeast cells tagged with all 20 green-red FRAME-tags. Left panels, GFP channel; middle panels, RFP channel; right panels, merged fluorescence overlay with brightfield channel. For each top and bottom panels are two different fields. Scale bar=20 μm. In FIG. 3 parts B and C, the automatic gating and analysis software previously developed for flow cytometry analysis of FRAME-tags was applied. In FIG. 3B, microscopy images containing 10⁴ cells were segmented and yEGFP and mCherry fluorescence values from each cell were extracted and plotted as a two-dimensional scatter plot, colored by event density. Individual FRAME-tag populations were programmatically assigned using software developed for flow cytometry in FIG. 3C. This allowed for simultaneous assignment of all 20 green-red FRAME-tag variants across more than 90% of the population.

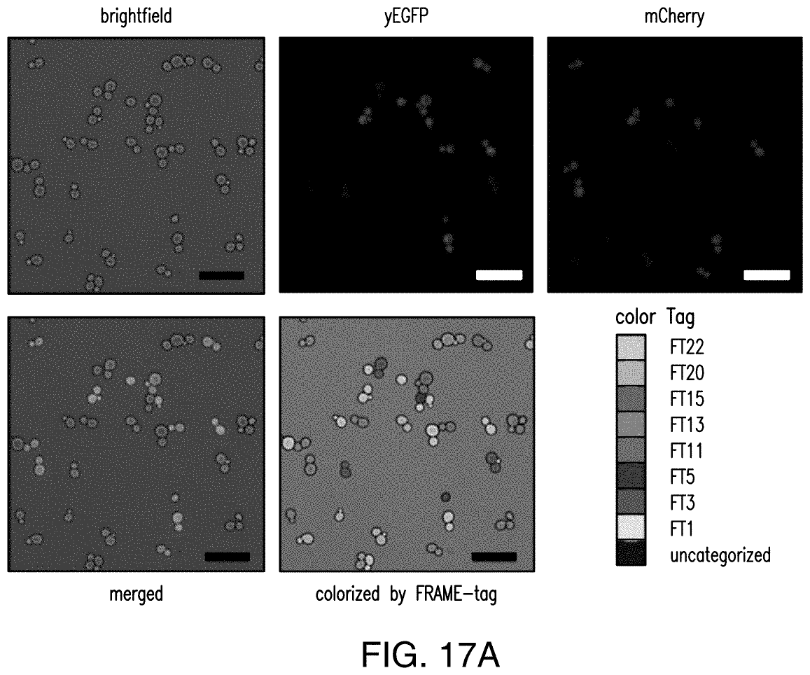

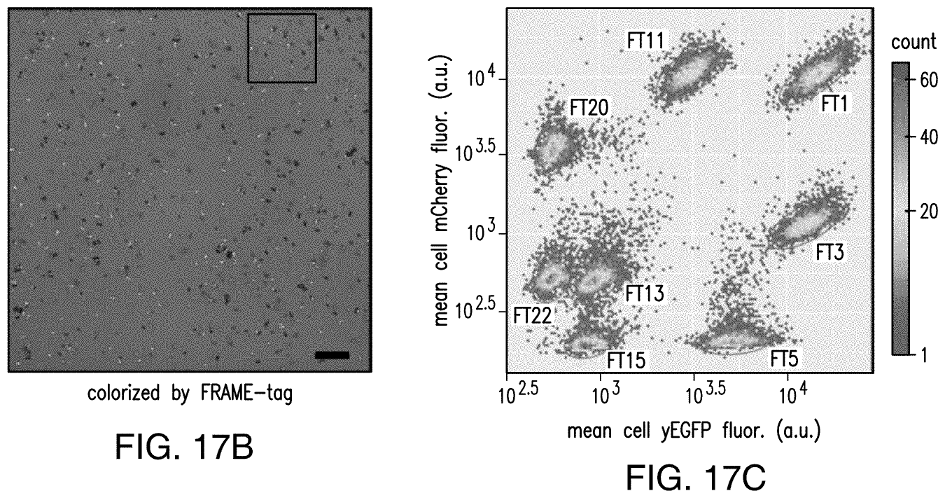

This is shown with 8 FRAME-tags in FIG. 17. FIG. 17A shows FRAME-tags imaged by brightfield, yEGFP, and mCherry with merged images. After the analysis as described in the Online Methods and identified using the automated gating pipeline (FIG. 14), yeast cells can be false colored. FIG. 17B shows one full wide-view image of colorized yeast. FIG. 17C shows grouping and analysis of individual FRAME-tags based on fluorescence microscopy, representing the composite data from 10 fields as shown in FIG. 17B and used for colorization.

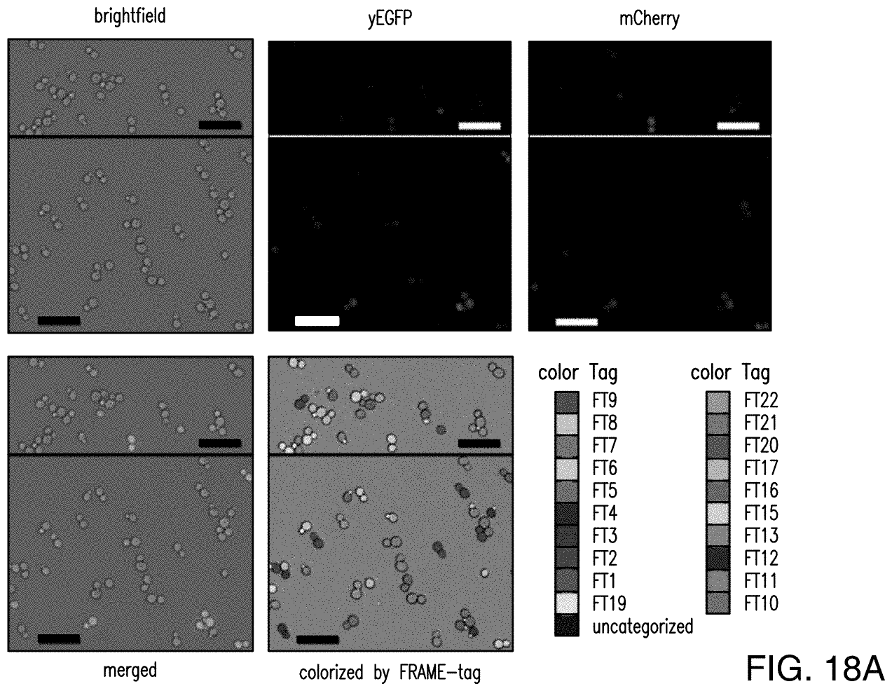

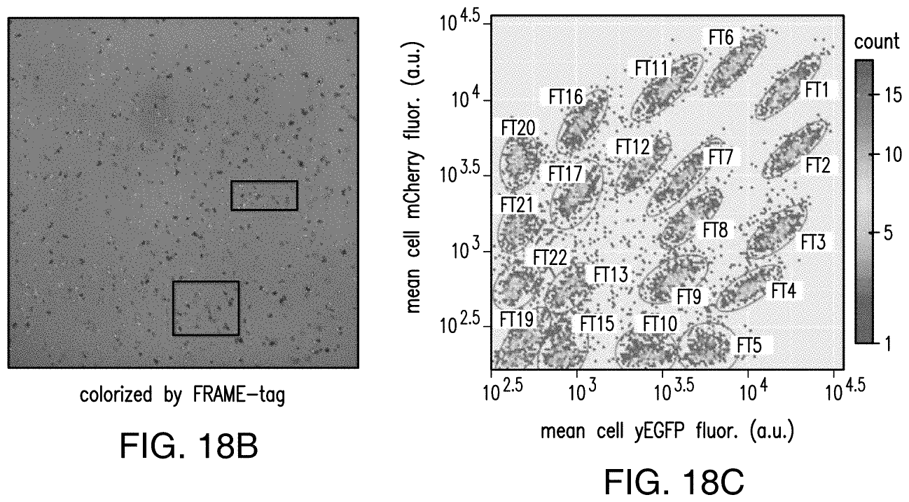

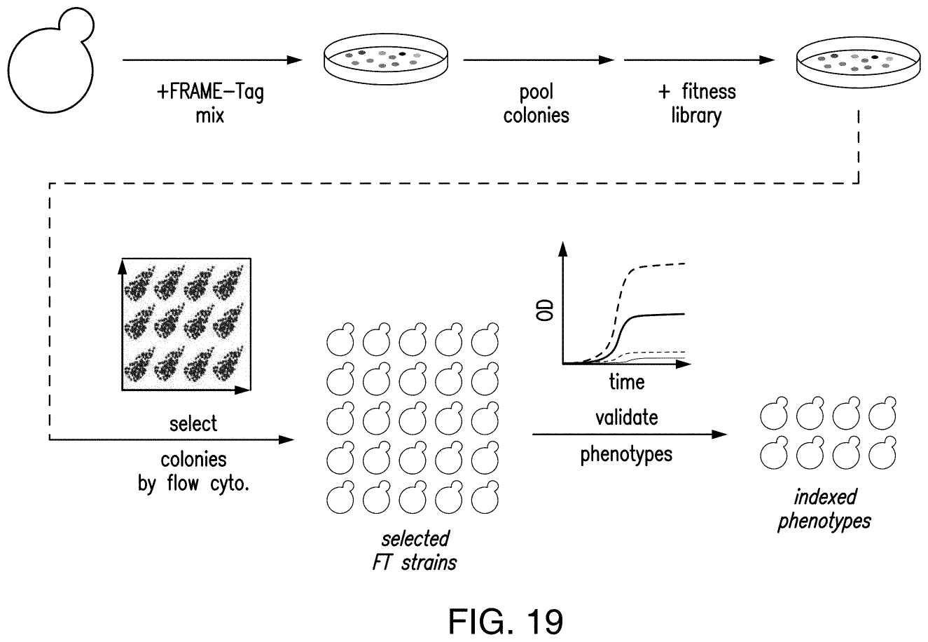

FIG. 18 depicts this result with 20 FRAME-tags. FIG. 18A shows FRAME-tags imaged by brightfield, yEGFP, and mCherry with merged images. After the analysis as described in the Online Methods and identified using the automated gating pipeline (FIG. 14), yeast cells can be false colored (FIG. 18B). FIG. 18B shows one full wide-view image of colorized yeast. FIG. 18C shows grouping and analysis of individual FRAME-tags based on fluorescence microscopy, representing the composite data from 10 fields as shown in FIG. 18B and used for colorization.

### Example 4: Tracking Frame-Tagged Cell Populations Through Time

### Yeast Community Tracking

A complex yeast library was constructed using the streamlined method that gives FRAME-tag indexed phenotypes with a minimal number of transformations (FIG. 19). FIG. 19 depicts a flowchart of the method. This method is designed to minimize DNA transformation steps. The mixed FRAME-tag DNA constructs can be co-transformed into the desired background strain. Barcoded transformants are directly pooled without tag identification, followed by transformation of pooled constructs or pre-screened libraries of constructs imparting a desired phenotype into the FRAME-tag strain mixture. Analysis is performed on individual colonies to first identify a set of strains with overrepresented FRAME-tag identities. The phenotypes of all members within this overrepresented set are identified and a subset of uniquely indexed phenotypes can be selected and used. Alternatively, pooled FRAME-tag DNA constructs can also be integrated into a preexisting pooled library of phenotypically variable strains followed by phenotype indexing as described.

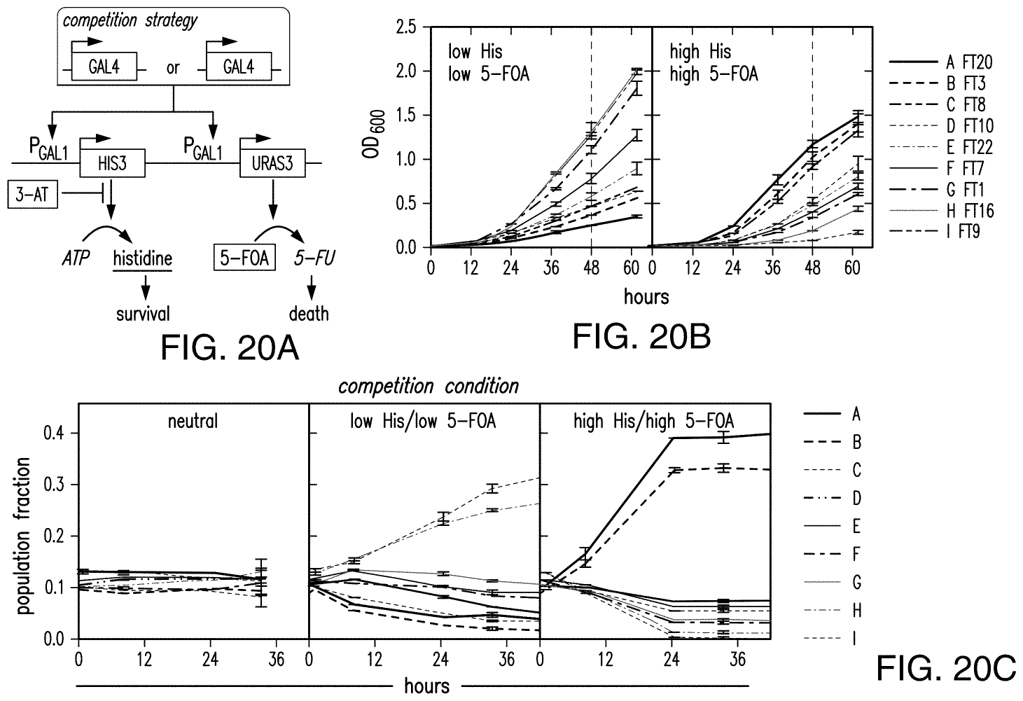

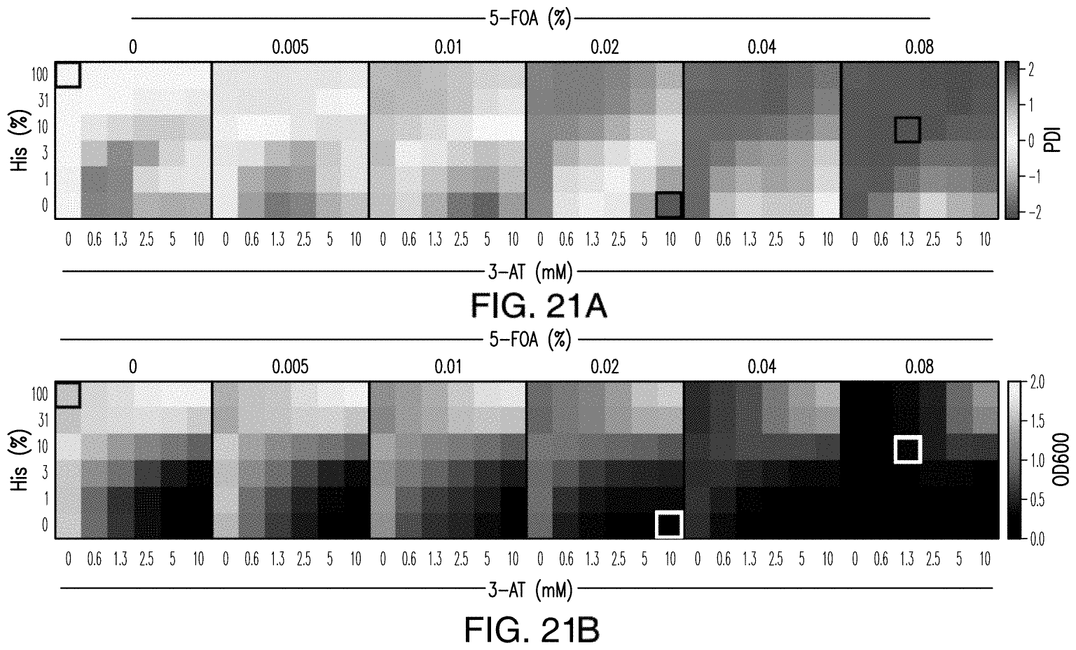

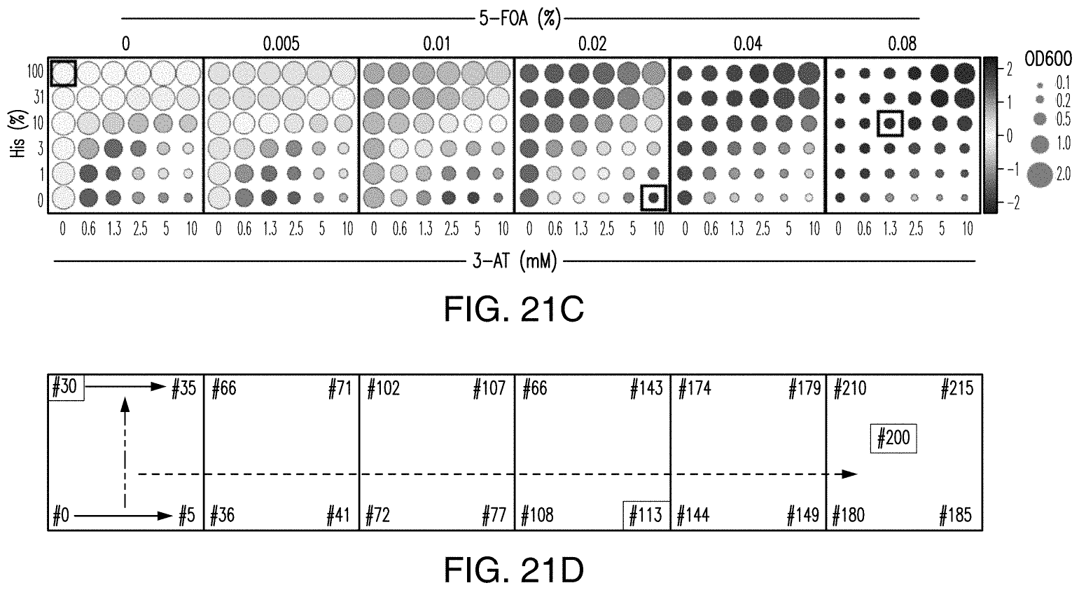

This yeast community was designed based on the MaV203 background strain, whose growth phenotype in the presence of varying concentrations of 5-FOA, histidine and 3-AT can be tuned based on Gal4 induction strength (FIG. 20). First, parent strain MaV203 was transformed with a DNA library of the 20 dual-FP FRAME-tag integrating constructs. The resulting transformants were pooled, grown overnight and transformed with a library of pRS424-SynGal4 plasmids (previously described pADH-Gal4(BD)-fs-Gal4(AD) construct library) that possess variable Gal4 transcriptional activity (Anzalone, A. V. et al., Nat. Methods 13, 453-458 (2016)). Individual transformants (harboring a FRAME-tag and SynGal4 plasmid) were screened by flow cytometry to identify a set of strains such that one to three of each FRAME-tag variant was included. The histidine and 5-FOA growth phenotypes of these strains were characterized by pre-culturing overnight in standard synthetic dropout medium (2% glucose) followed by inoculation into 96-well plates containing Low His/Low 5-FOA selective media (2% glucose, 0 μM His, 5 mM 3-AT) or High His/High 5-FOA selective media (2% glucose, 130 μM His, 0.1% 5-FOA) to an OD₆₀₀ of 0.01. Cultures were incubated at 30° C. and 800 RPM for 48 hours. Nine strains were selected for the final yeast community so that each member contained a unique FRAME-tag and a unique growth phenotype (see Table 9 for SynGal4 sequences). For co-culture experiments, each strain was pre-cultured individually overnight in standard synthetic dropout media (2% glucose) at 30° C. and 800 RPM. OD₆₀₀ values were measured and strains were mixed to yield a combined community with an equal proportion of all strains. For the time-course assays displayed in FIG. 3B, the community was used to inoculate cultures to an OD₆₀₀ of 0.01 in the following media conditions; non-selective: 130 μM His, 0 mM 3-AT, 0% 5-FOA; selection 1: 0 μM His, 5 mM 3-AT, 0% 5-FOA; selection 2: 130 μM His, 0 mM 3-AT, 0.1% 5-FOA. Communities were grown at 30° C. and 250 RPM and sampled at the indicated time points. The composition of the community (strains A through I) was determined by flow cytometry and automatic gating software, with each member's prevalence calculated as the events assigned to that member divided the total events assigned to all members, excluding unassigned events. To map the growth phenotype landscape of the community, mixed cultures were inoculated at an OD₆₀₀ of 0.01 into 216 individual wells (in 96-well format) containing unique growth mediums (covering a 6×6×6 matrix representing all combinations of Histidine, 3-AT and 5-FOA concentrations in a base synthetic dropout medium, see FIG. 21). Cultures were incubated for 24 hours at 30° C. and 800 RPM, the OD₆₀₀ was recorded, plates were placed on ice, and cultures were analyzed by flow cytometry within two hours. Community composition was quantified as above. To characterize dynamic community restructuring caused by selective pressure in real time, the yeast community was transitioned between pairs of conditions chosen from the phenotype map as indicated (FIG. 3 and FIG. 21). The yeast community was prepared in standard synthetic dropout medium (2% glucose) as described above. Conditions used were as follows; condition #113 (blue): 0 μM His, 10 mM 3-AT, 0.02% 5-FOA); condition #200 (red): 13 μM His, 1.3 mM 3-AT, 0.08% 5-FOA; condition #30 (grey): 130 μM His, 0 mM 3-AT, 0% 5-FOA. The community was inoculated into 10 mL of the initial condition at an OD₆₀₀ of 0.01 and incubated at 30° C. and 250 RPM for 24 hours. Each pre-conditioned community was then pelleted (1 min, 15 k RPM), washed once in 1.5 mL of the second condition medium then inoculated into 10 mL of the second condition medium at an OD₆₀₀ of 0.03 and incubated at 30° C. and 250 RPM. 100 μL samples were analyzed by flow cytometry (see above) every 20 min for a period of 13 hours.

### Results

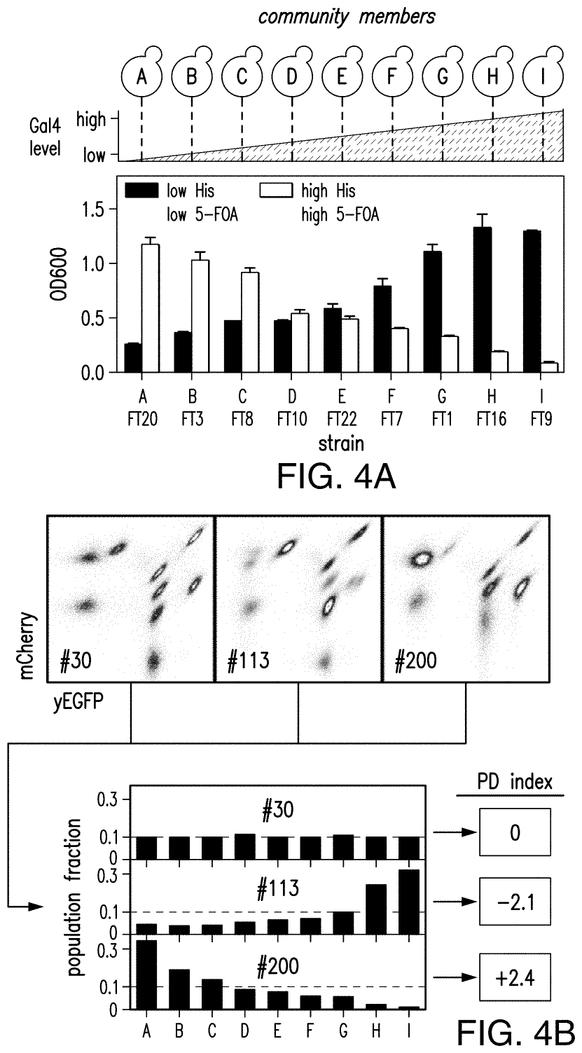

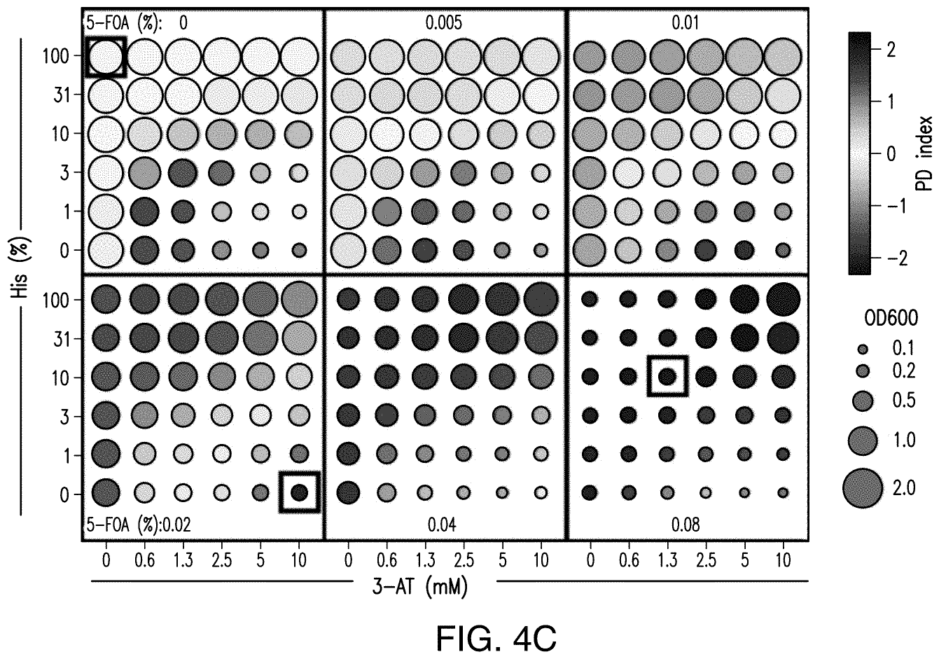

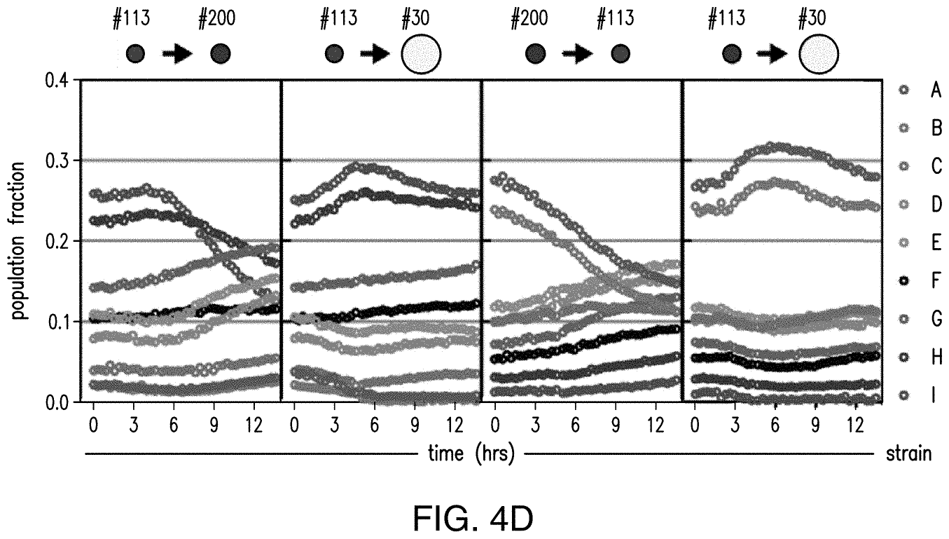

After establishing the fluorescent barcode palette and efficient analytical tools, FRAME-tags were applied for long-term, continuous tracking of cell populations composed of multiple distinct cell types. First, a streamlined workflow was formulated for introducing FRAME-tags into new host yeast strains (FIG. 19). Using this workflow, nine FRAME-tagged strains were created that differ in their ability to produce an essential resource, histidine, while simultaneously incurring a proportional sensitivity to the growth inhibiting 5-fluoroorotic acid (5-FOA) (FIG. 20). FIG. 20 depicts the data from the multi-strain competitive community. FRAME-tags were introduced into a background strain of MaV203. The competition strategy was designed such that growth would be dependent on two factors within the host strain, namely His3 and Ura3 expression. Growth selection was modified with exogenously added factors, namely histidine, 3-aminotriazole (3-AT), and 5-fluoroorotic acid (5-FOA). His3 and Ura3 production are dependent on Gal4 transcriptional activity. High production of His3 allows for cells to grow in the absence of histidine and the presence of 3-AT. High Ura3 expression causes production of the antimetabolite 5-FU in the presence of 5-FOA, which inhibits growth. To impart each strain with a different level of Gal4 activity, previously developed ratiometric Gal4 constructs containing frameshift modules between the N-terminal DNA binding domain and the C-terminal activation domain were implemented (see Table 4 and Table 9) (FIG. 20A) (Anzalone, A. V. et al., Nat. Methods 13, 453-458 (2016)). Overall Gal4 activity is a function of frameshift efficiency. Frameshift sequences were screened in a library fashion on plasmids. Plasmids with desirable phenotypes were transformed into pooled FRAME-tag strains as discussed above. FIG. 20B shows the results from a set of strains with uniquely indexed growth phenotypes which were selected and characterized in single-strain experiments. OD₆₀₀ values were measured over 60 hours at 30° C. and 800 RPM; error bars indicate s.d. from 2 replicates. Low His/Low 5-FOA=2% glucose, -His, 5 mM 3-AT; High His/High 5-FOA=2% glucose, 130 μM His, 0.1% 5-FOA. Dashed lines mark data used in FIG. 4a. FIG. 20C shows time courses of the community grown in non-selective media, or Low His/Low 5-FOA (2% glucose, -His, 5 mM 3-AT) or High His/High 5-FOA (2% glucose, 130 μM His, 0.1% 5-FOA), analyzed by flow cytometry using FRAME-tags to determine the respective population fraction of each community member; error bars indicate s.e.m. from 3 replicates. Each of the nine strains displays a distinct growth advantage or disadvantage when cultured independently in two standard selective conditions (FIG. 4A and FIG. 20).

The composition of this synthetic community was tracked in various conditions using flow cytometry-based FRAME-tag identification (FIG. 4B). Given the speed and ease with which FRAME-tagged strains are analyzed in this manner, a rapid assessment of the community's growth and composition across 216 distinct culture conditions was made, and a population distribution (PD) index for each (FIG. 4C and FIG. 21) assigned. First, FIG. 4 shows monitoring microbial community dynamics in real-time with FRAME-tags. FIG. 4A is a schematic showing a synthetic yeast community made from individual strains with varying levels of Gal4 expression leading to distinct growth phenotypes in selective conditions (see above); each strain was barcoded with a unique FRAME-tag (A-I). FIG. 4B shows flow cytometry analysis of the mixed community used to extract population fractions of each community member under various culture conditions (#30, #113, #200) and summarized as a Population Distribution (PD) index (discussed further below). FIG. 4C shows the PD index for the community in 216 culture conditions after 24 hours growth as analyzed by flow cytometry. The total growth of the community was determined by OD600. Boxes indicate conditions #30, #113 and #200. FIG. 4D shows community response to abrupt changes in culture conditions (#30, #113, #200) as evaluated for four distinct transitions. Cultures were sampled and quantified at 20-minute intervals by flow cytometry using FRAME-tags to determine the respective population fraction of each community member.

FIG. 21 shows how the yeast community was profiled in a high-throughput fashion. The resulting community composition was assessed after 24 hours cultured in 216 conditions varying in concentrations of histidine (His), 5-fluoroorotic acid (5-FOA), and 3-aminotriazole (3-AT) (FIG. 21A-C). His levels are shown as the percent of standard SC media, with 100% equaling 130 uM. FRAME-tags were used to determine all individual strain population fractions. For visual representation of the selective pressure imparted by conditions, the Population Distribution Index (PDI) was determined for each condition. Defining the set containing all nine strains as S={A, B, C, D, E, F, G, H, I}, the set X={−4, −3, −2, −1, 0, 1, 2, 3, 4}, and a function ƒ that maps S to X such that {A, B, . . . I}→{−4,−3, . . . 4}. Note that S is ordered by decreasing fitness in the High His/High 5-FOA standard selection condition and so this mapping gives a relative numerical value of strain identity and phenotype. The PDI was determined from the culture composition according to the formula below:

$$PDI = {\sum }_{s ∈ S} {X}_{s} \cdot {P}_{s}$$

where Xₛ is the mapped integer value for Sₛ and Pₛ is the measured population fraction of strains. PDI takes values between −4 and 4. The results of 216 growth conditions plotted as a heatmap of PDI are shown in FIG. 21A, with red tiles indicating low PDI values and blue tiles indicating high PDI values. In general, red conditions favor strains with low Gal4 expression, while blue conditions favor strains with high Gal4 expression. FIG. 21B shows absolute growth for the entire culture (all strains) in each of the 216 growth conditions, represented as a heat map with lighter color representing higher measured OD₆₀₀. FIG. 21C shows combined data from FIG. 21A and FIG. 21B to generate an overall community state map. Color represents PDI, as in the top panel; circle diameter represents growth and is proportional to the measured final OD₆₀₀ of the bulk culture in that condition. FIG. 21D shows a schematic of the condition numbering from 0 to 215 as shown starting from the lower left corner of the array and continuing as shown by the black then grey then dashed arrows. Conditions used in FIG. 4d are labeled in all panes with square outlines.

An assessment was made if FRAME-tags would be useful for tracking subpopulation dynamics over time. To test this, an abrupt transition of the community between different culture conditions was made, and changes in strain abundance measured. This provided a detailed readout of subpopulation growth trajectories in real-time (FIG. 4D). Surprisingly, the results showed temporally coupled growth surge and collapse that would have been undetectable by certain techniques that only capture a single snapshot of the population.

TABLE 9
DNA sequences of SynGal4 constructs
for competitive community tracking.

| Part | Sequence |
|---|---|
| Gal4-BD | ATGAAGCTACTGTCTTCTATCGAACAAGCATGCGATATT |
| | TGCCGACTTAAAAAGCTCAAGTGCTCCAAAGAAAAACCG |
| | AAGTGCGCCAAGTGTCTGAAGAACAACTGGGAGTGTCGC |
| | TACTCTCCCAAAACCAAAAGGTCTCCGCTGACTAGGGCA |
| | CATCTGACAGAAGTGGAATCAAGGCTAGAAAGACTGGAA |
| | CAGCTATTTCTACTGATTTTTCCTCGAGAAGACCTTGAC |
| | ATGATTTTGAAAATGGATTCTTTACAGGATATAAAAGCA |
| | TTGTTAACAGGATTATTTGTACAAGATAATGTGAATAAA |
| | GATGCCGTCACAGATAGATTGGCTTCAGTGGAGACTGAT |
| | ATGCCTCTAACATTGAGACAGCATAGAATAAGTGCGACA |
| | TCATCATCGGAAGAGAGTAGTAACAAAGGTCAAAGACAG |
| | TTGACTGTATCG |
| Gal4-AD | GCCAATTTTAATCAAAGTGGGAATATTGCTGATAGCTCA |
| | TTGTCCTTCACTTTCACTAACAGTAGCAACGGTCCGAAC |
| | CTCATAACAACTCAAACAAATTCTCAAGCGCTTTCACAA |
| | CCAATTGCCTCCTCTAACGTTCATGATAACTTCATGAAT |
| | AATGAAATCACGGCTAGTAAAATTGATGATGGTAATAAT |
| | TCAAAACCACTGTCACCTGGTTGGACGGACCAAACTGCG |
| | TATAACGCGTTTGGAATCACTACAGGGATGTTTAATACC |
| | ACTACAATGGATGATGTATATAACTATCTATTCGATGAT |
| | GAAGATACCCCACCAAACCCAAAAAAAGAGTAA |
| fsA | GGATCCGGCAGTGGTTCTGGCGCACTACAAGGACGACGA |
| | CACAAGACTTTAAACTAGTTGACGCGGGTCTAGCCGTTC |
| | CCGCGTTAAACGTACTAGAAGGCGGTTCTATGGGAATGT |
| | CTGGAAAGCTT |
| fsB | GGATCCGGCAGTGGTTCTGGCGCACTACAAGGACGACGA |
| | CACAAGACTTTAAACTAGTTGACGCGGGTTTAAGTACGG |
| | TCGCGTTAAACTCGCTAGAAGGCGGTTCTATGGGAAATG |
| | TCTGGAAAGCTTGCCAATTTTAA* |
| fsC | GGATCCGGCAGTGGTTCTGGCGCACTACAAGGACGACGA |
| | CACAAGACTTTAAACTAGTTGACGCGAGTCTAATGTCTG |
| | CCGCGTTAAACTCGCTAGAAGGCGGTTCTATGGGAATGT |
| | CTGGAAAGCTT |
| fsD | GGATCCGGCAGTGGTTCTGGCGCACTACAAGGACGACGA |
| | CATAAGACTTTAAACTAGTTGACGCGGTTCTAGGTCAAC |
| | CCGCGTTAAACATCCTAGAAGGCGGTTCTATGGGAATGT |
| | CTGGAAAGCTT |
| fsE | GGATCCGGCAGTGGTTCTGGCGCACTACAAGGACGACGA |
| | CACAAGACTTTAAACTAGTTGACGCGGATCTAGGAGGTA |
| | TCGCGTTAAACAGCCTAGAAGGCGGTTCTATGGGAATGT |
| | CTGGAAAGCTT |
| fsF | GGATCCGGCAGTGGTTCTGGCGCACTACAAGGACGACGA |
| | CACAAGACTTTAAACTAGTTGACGCGTCGCTACCAGTGG |
| | GCGCGTTAAATGATCTAGAAGGCGGTTCTATGGGAATGT |
| | CTGGAAAGCTT |
| fsG | GGATCCGGCAGTGGTTCTGGCGCACTACAAGGACGACGA |
| | CACAAGACTTTAAACTAGTTGACGCGGGACTAGGCTTTG |
| | CCACGTTAAACAGCCTAGAAGGCGGTTCTATGGGAATGT |
| | CTGGAAAGCTT |
| fsH | GGATCCGGCAGTGGTTCTGGCGCACTACAAGGACGACGA |
| | CACAAGACTTTAAACTAGTTGACGCGGGTCTAGCACCTC |
| | GCGCGTTAAACGTGCTAGAAGGCGGTTCTATGGGAATGT |
| | CTGGAAAGCTT |
| fsI | GGATCCGGCAGTGGTTCTGGCGCACTACAAGGACGACGA |
| | CACAAGACTTTAAACTAGTTGACGCGGGACTAGGCATAT |
| | GCGCGTTAAACAACCTAGAAGGCGGTTCTATGGGAATGT |
| | CTGGAAAGCTT |

SynGal4 constructs are assembled as Gal4-BD • fs • Gal4-AD. Frameshift slippery sites are shown underlined.
*This sequence is mutated and contains a stop codon (bold) in the -1 frame shortly following the canonical fs module sequence.

### Example 5: Tracking Frame-Tagged Cell Populations with Both Frame-Tags and Other Fluorescent Reporters

### Multiplexed Transcriptional Profiling

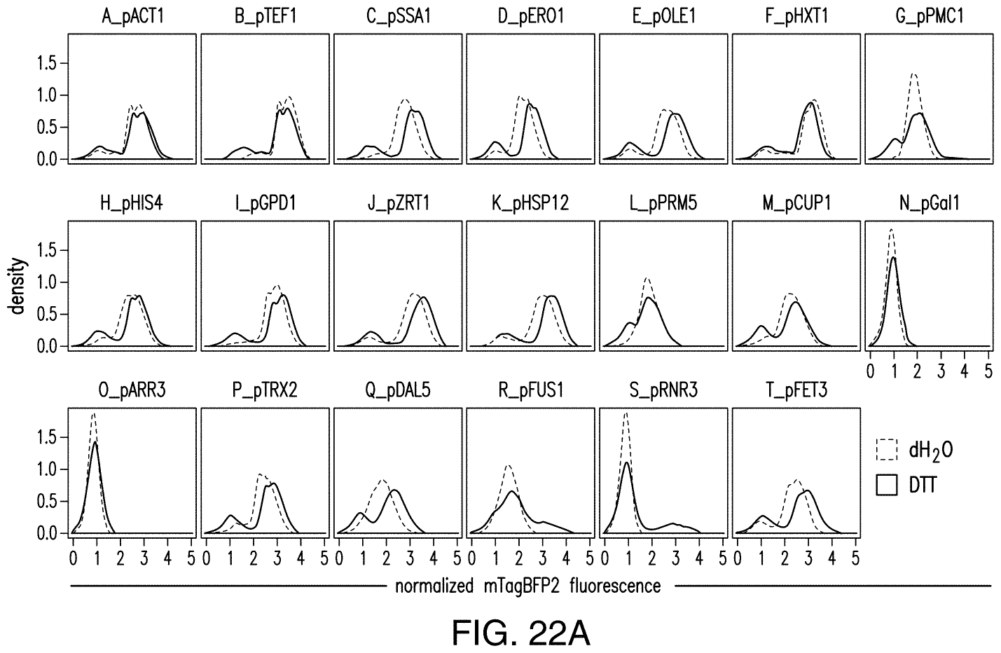

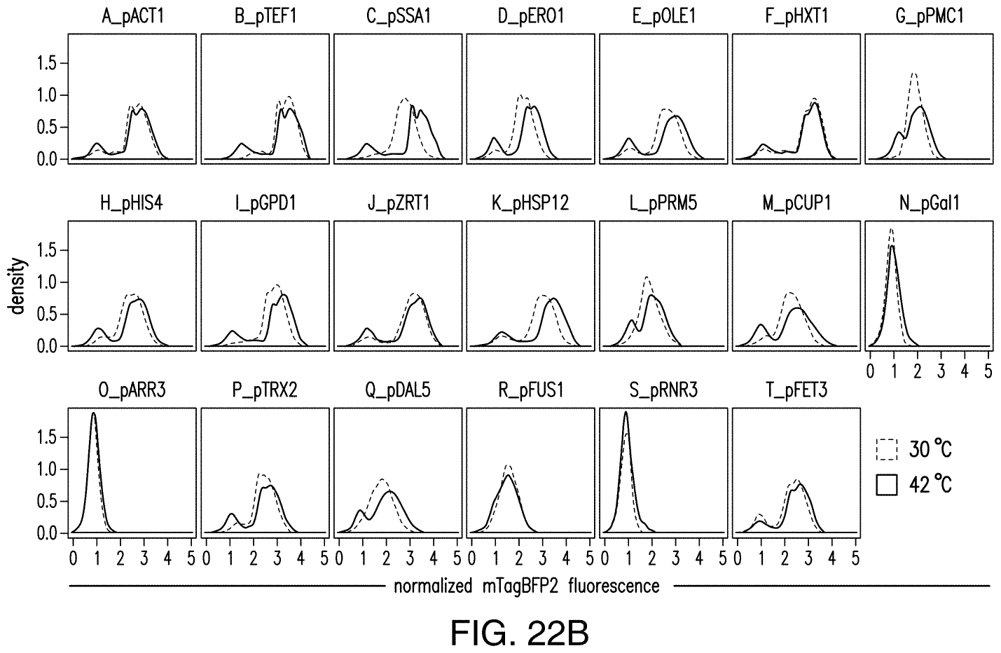

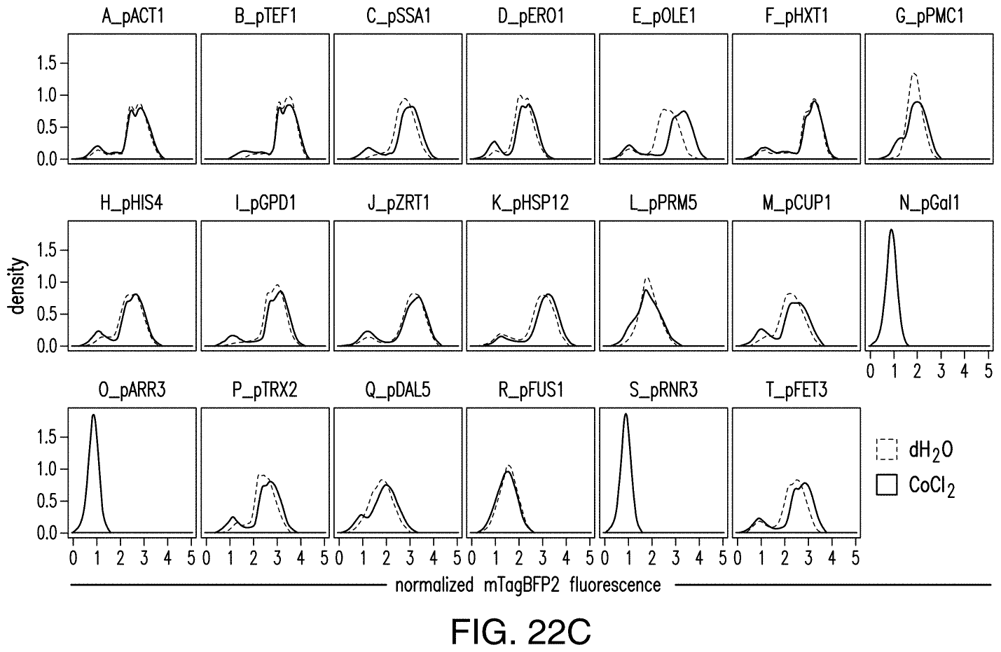

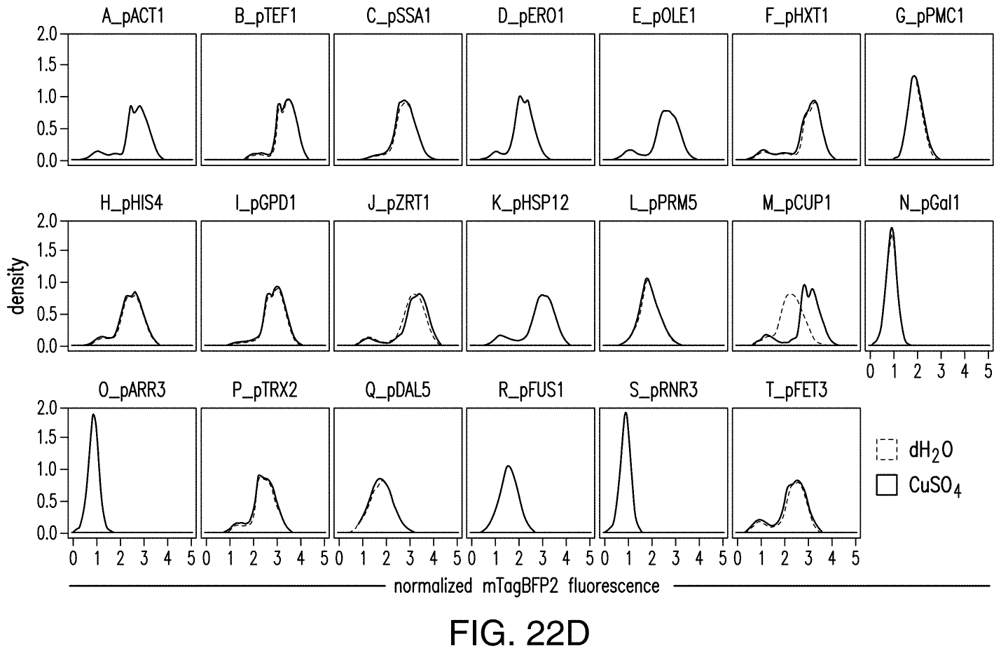

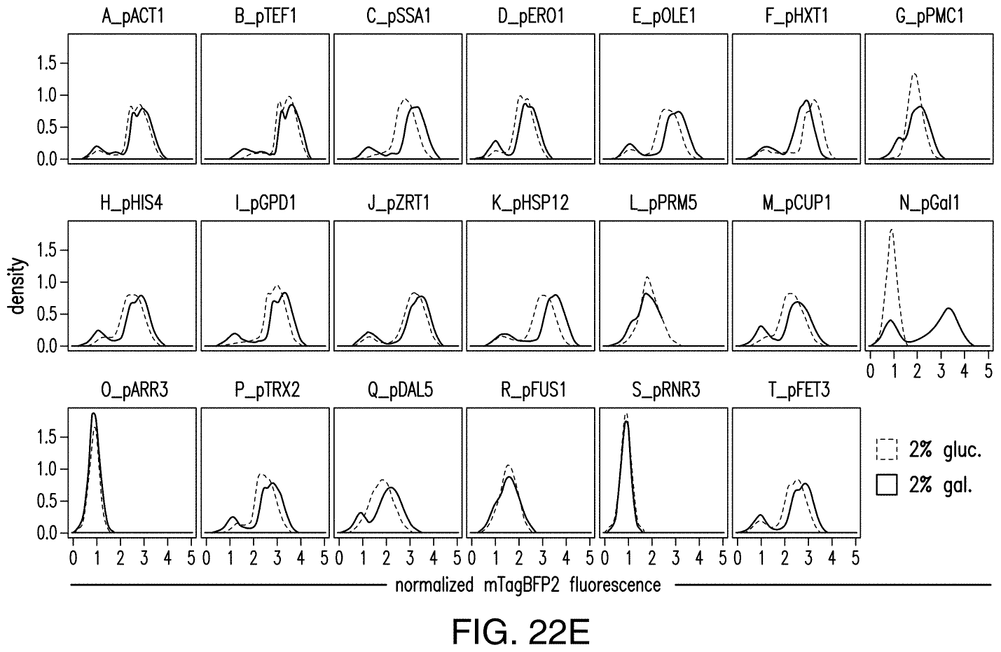

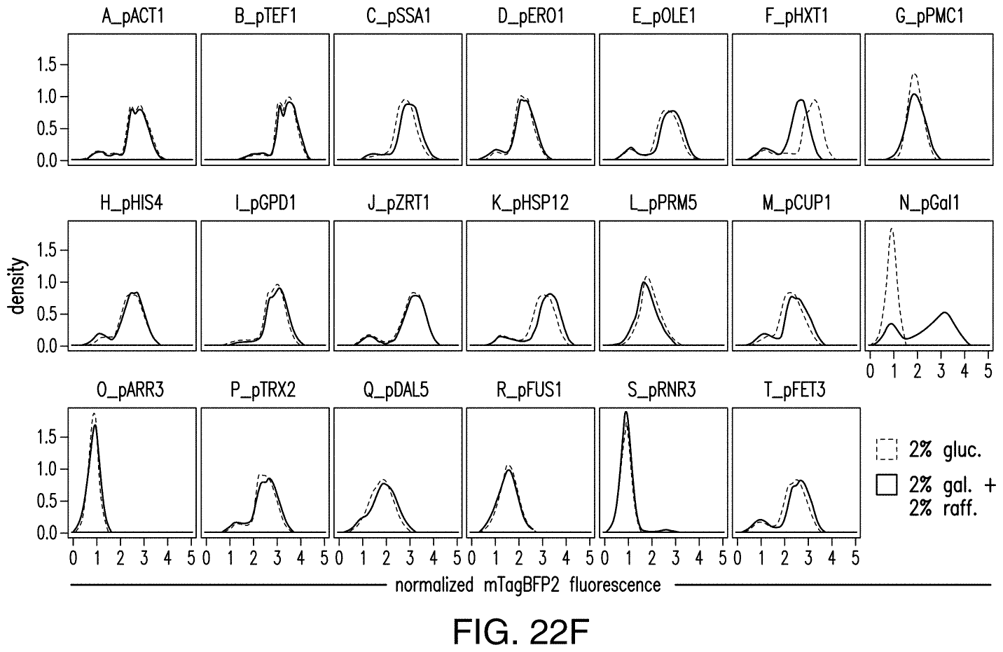

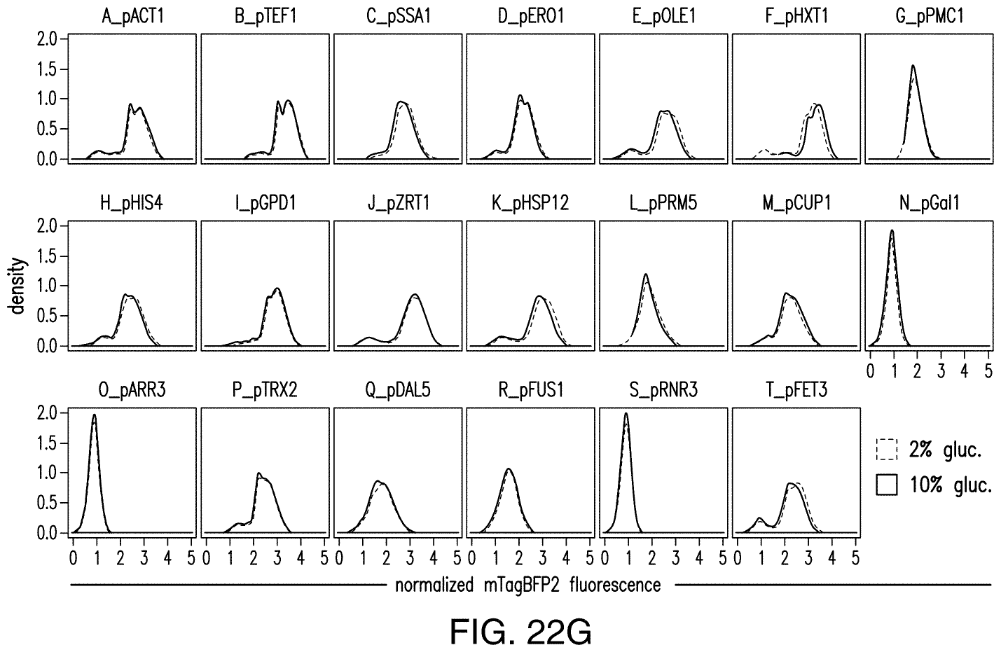

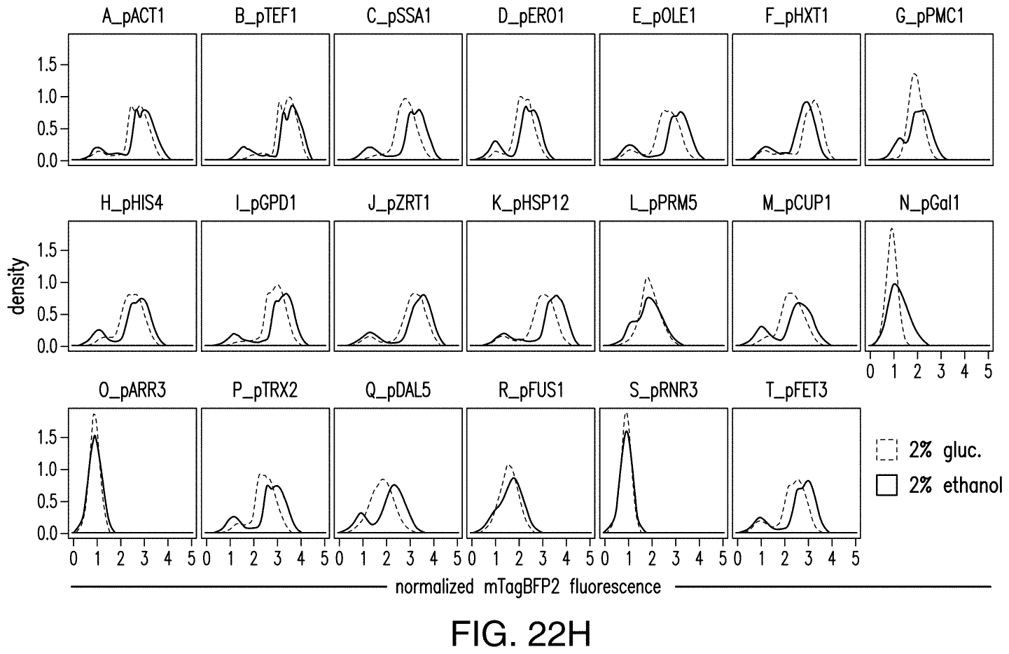

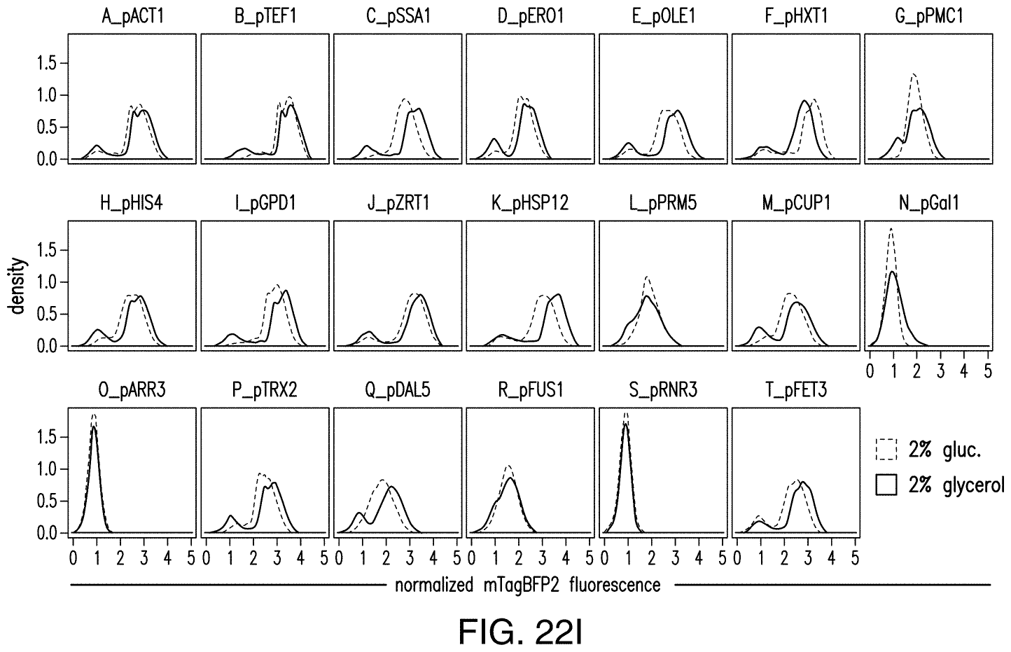

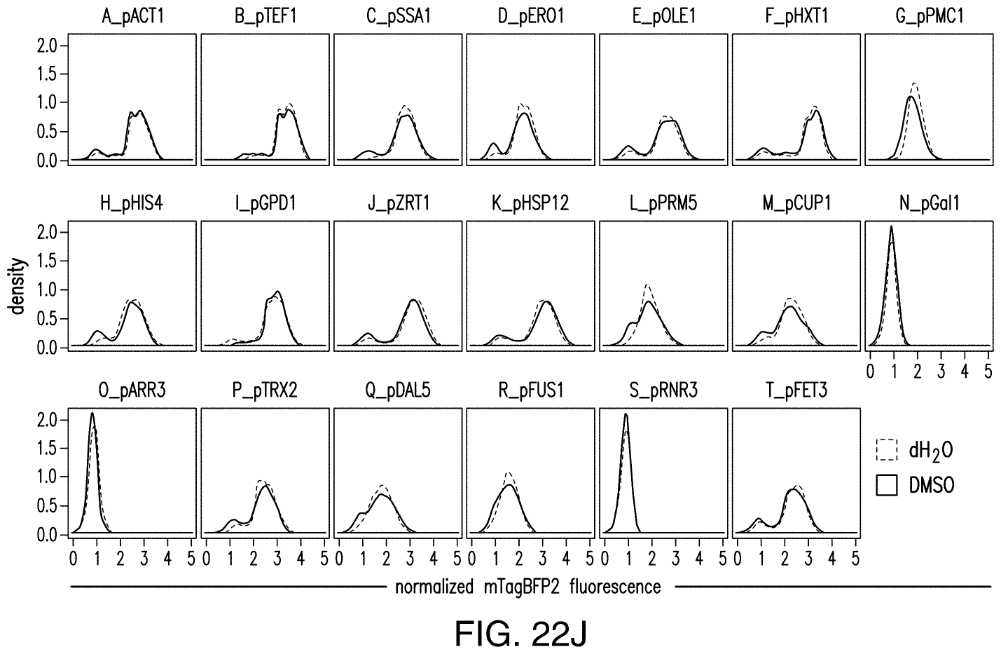

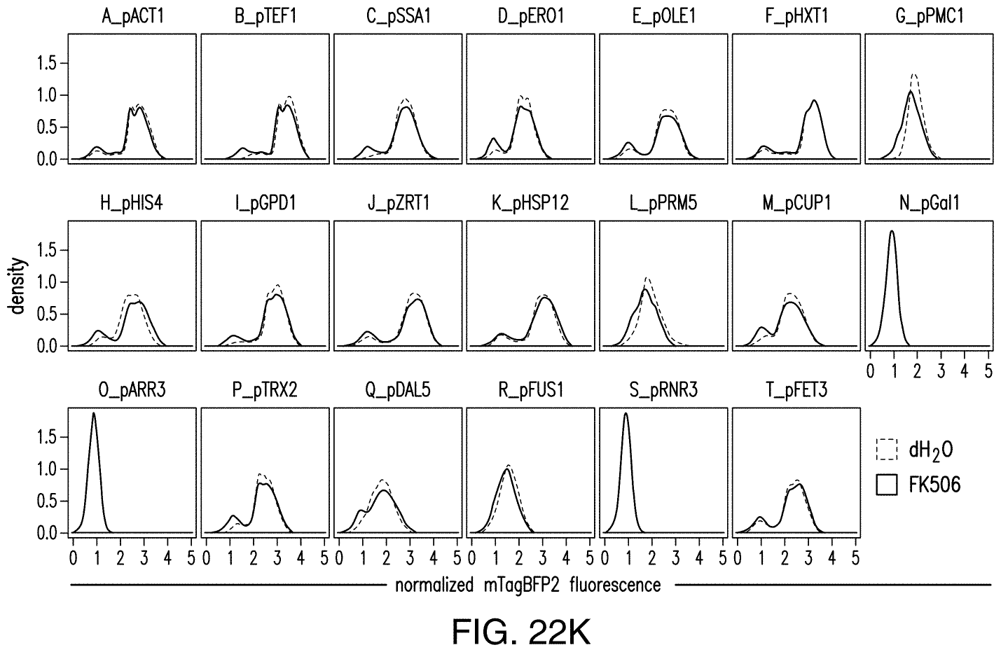

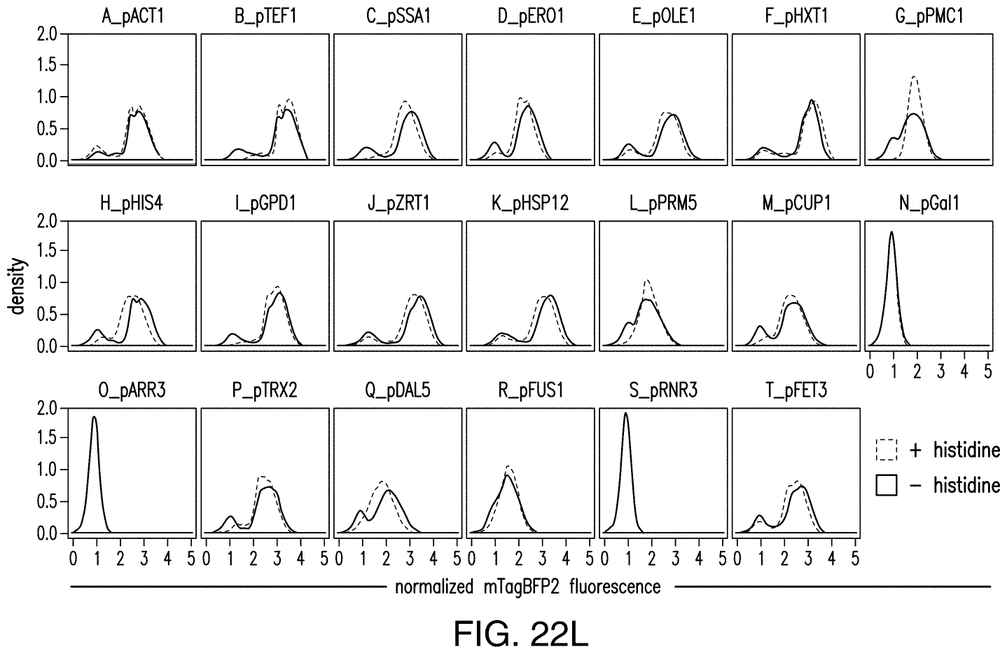

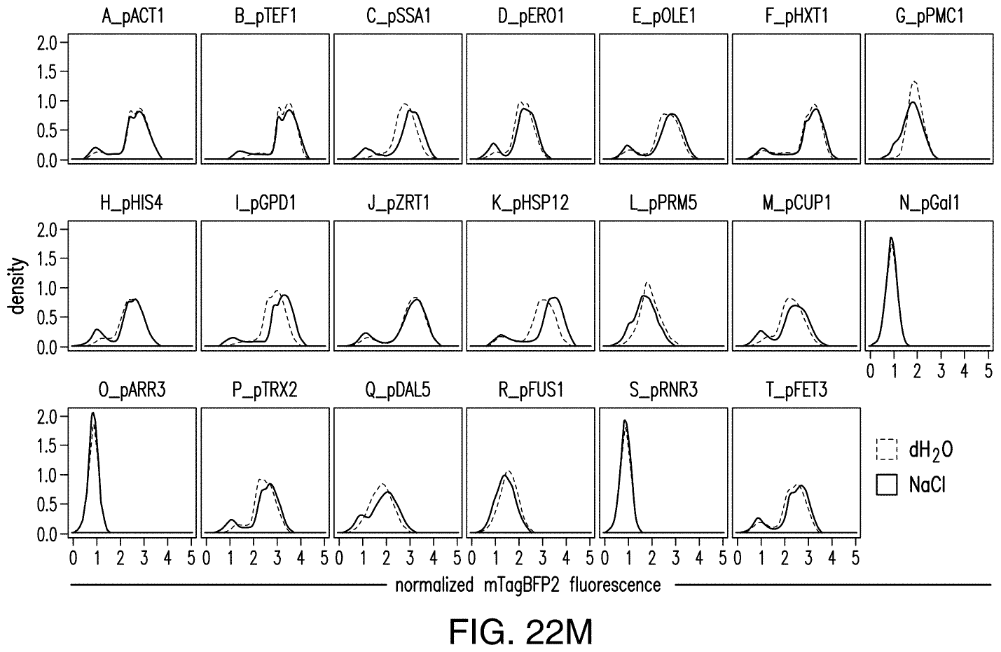

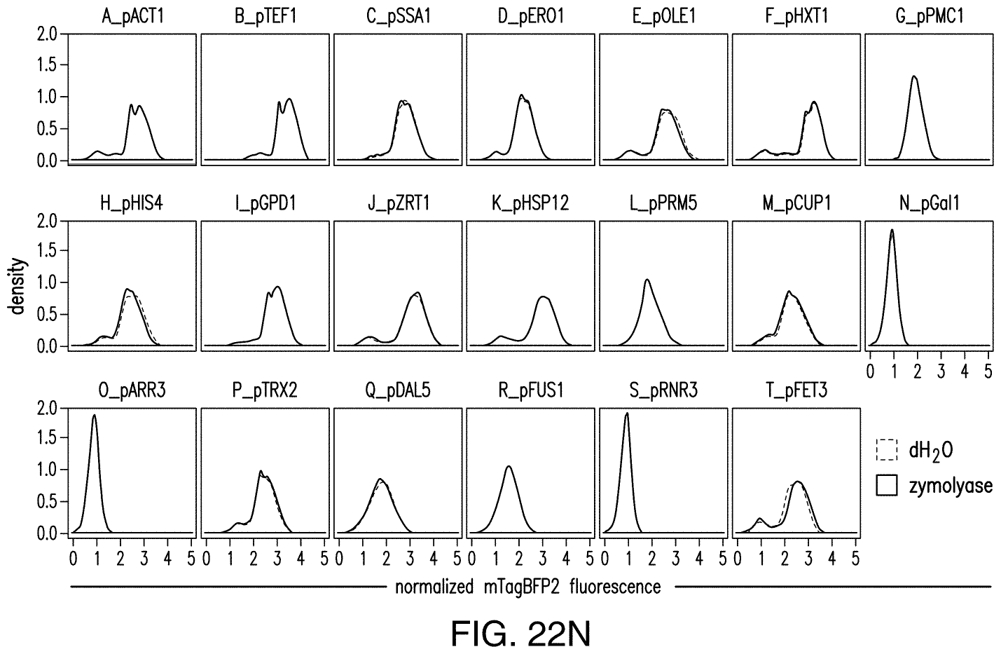

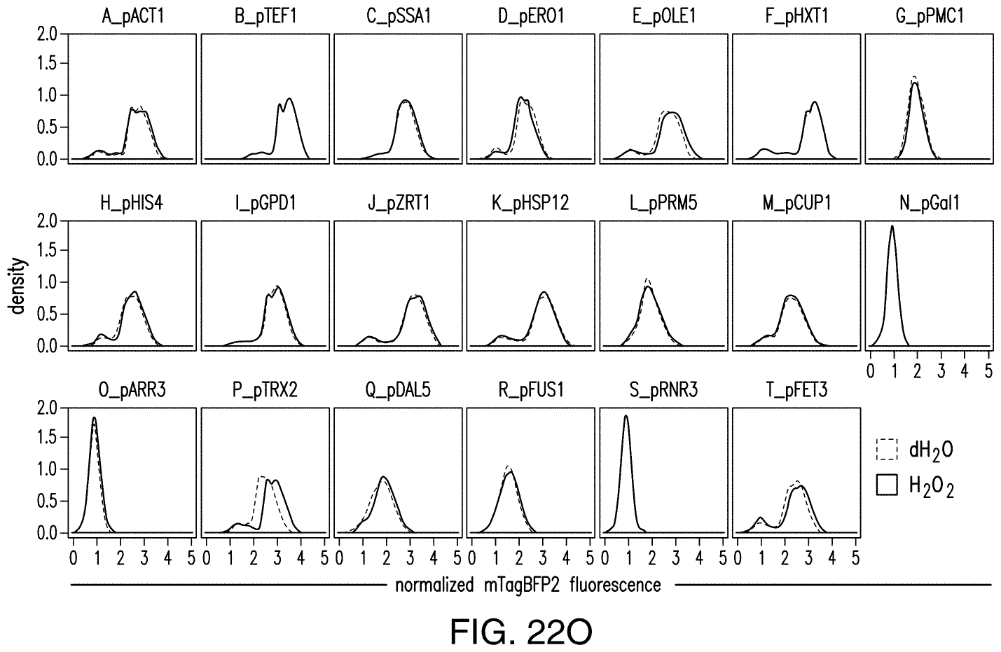

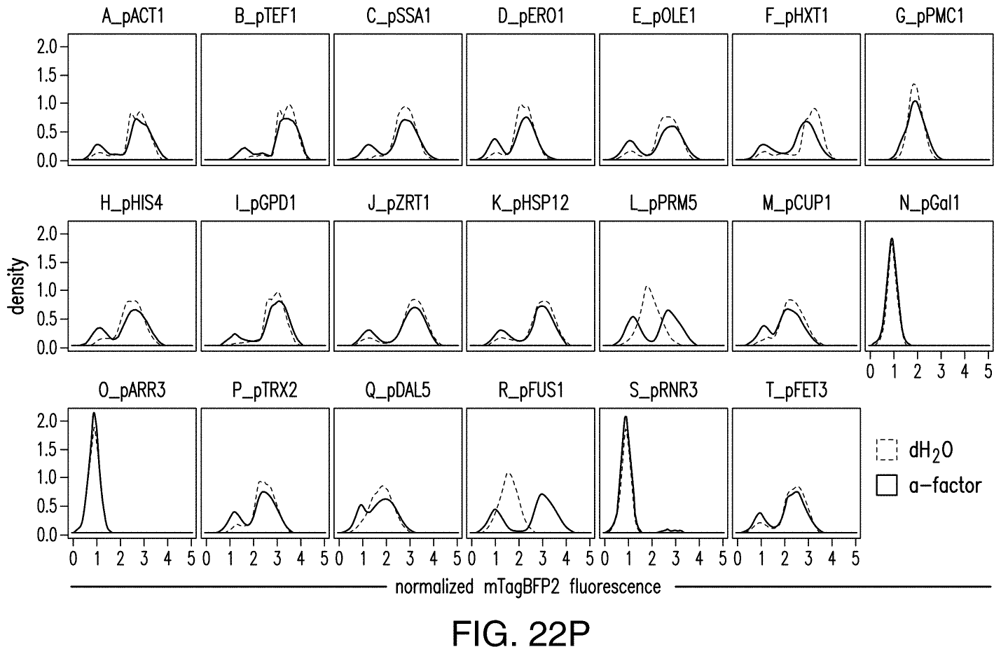

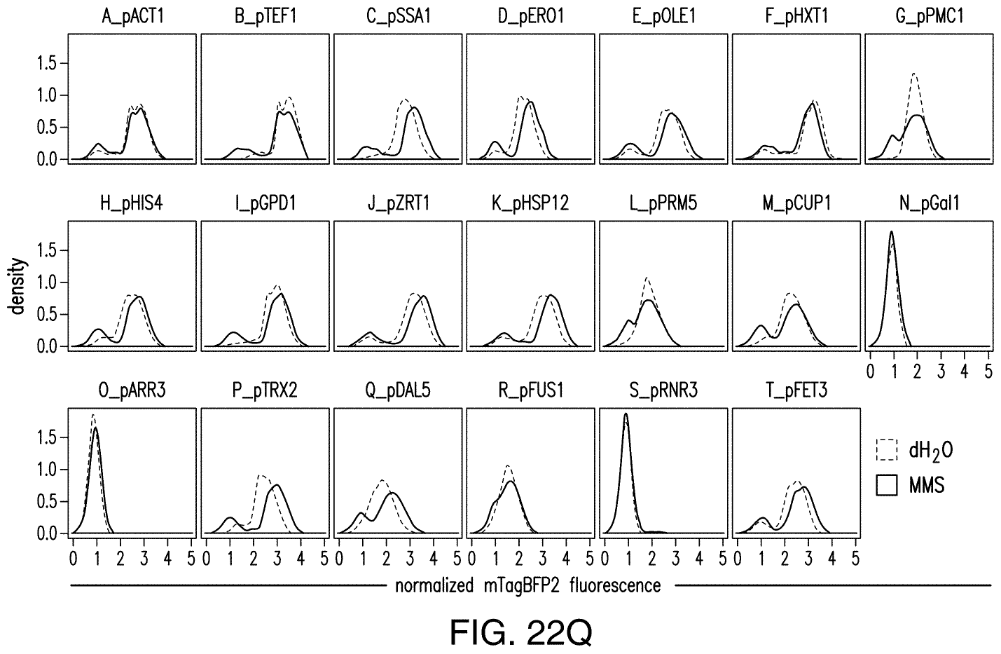

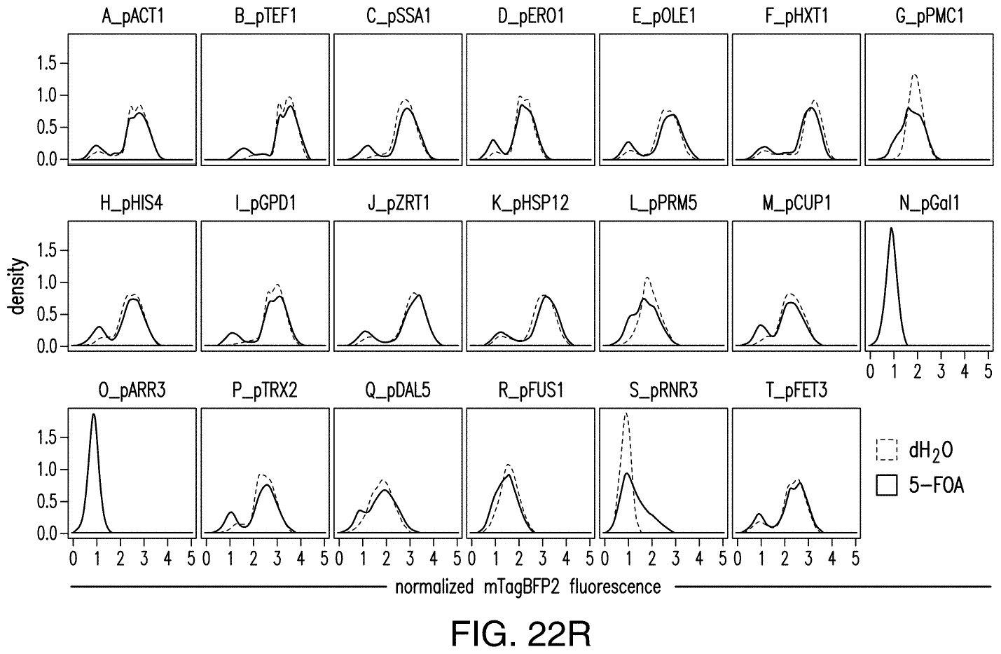

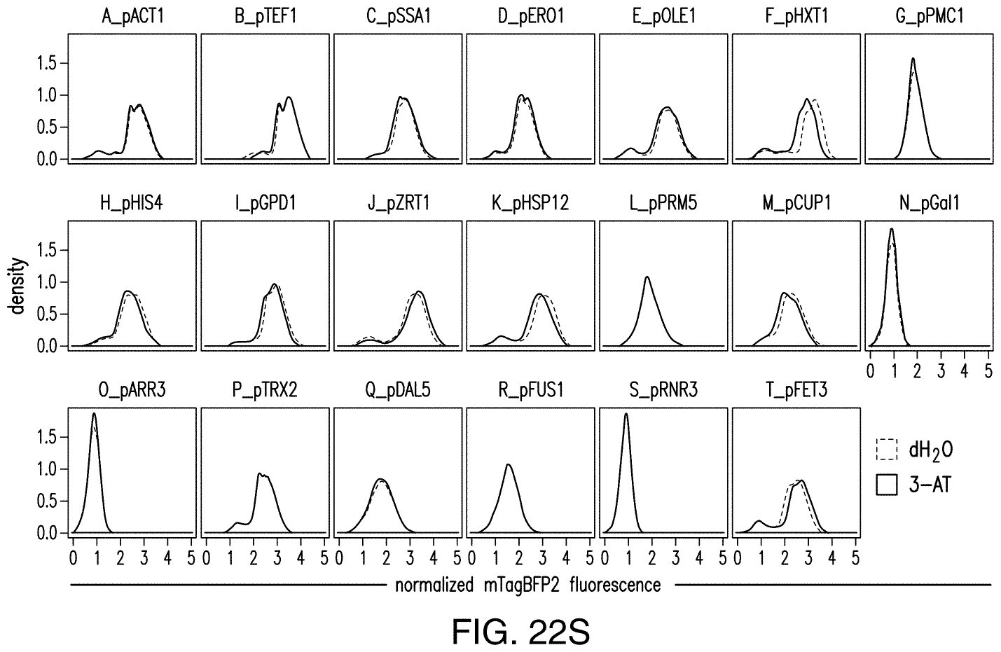

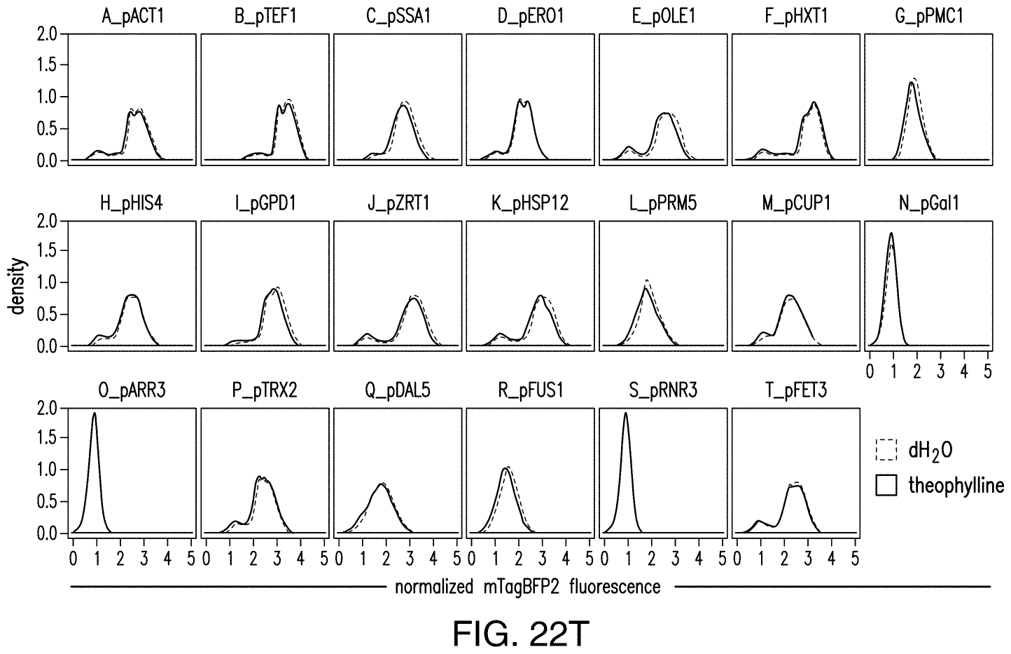

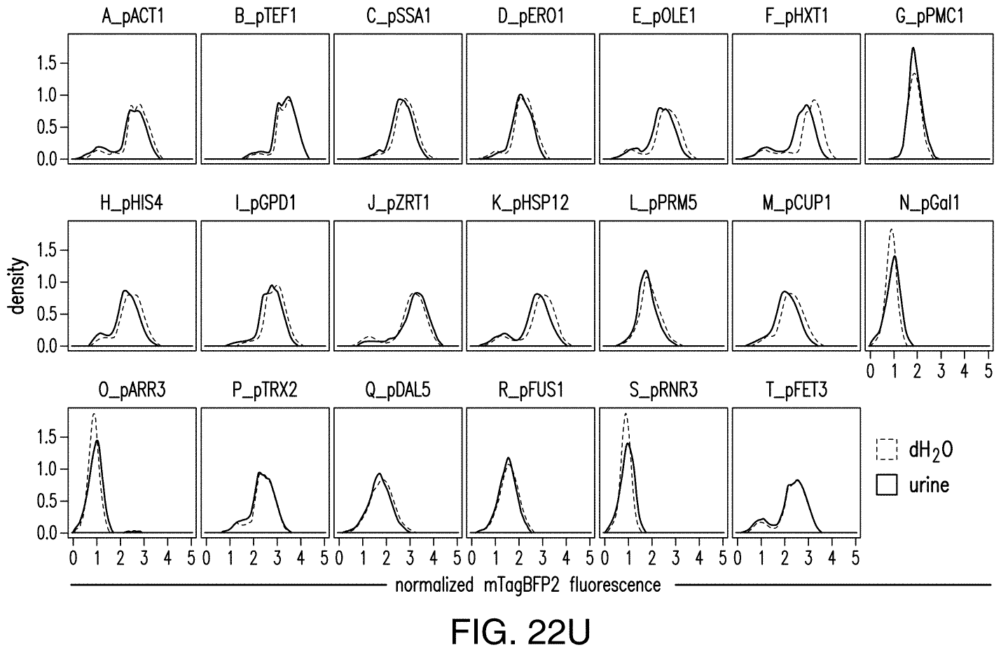

Native yeast promoters were cloned upstream of an mTagBFP2 expression construct within a pRS416 plasmid backbone (see Table 5 and Table 8). Sequence confirmed reporter plasmids were then transformed into separate FRAME-tagged Fy251 strains. Strains were individually pre-cultured overnight in 96-well plates in standard synthetic dropout media (2% glucose) at 30° C. and 800 RPM. Culture density was measured using the OD₆₀₀, and strains were combined to yield a mixed culture containing an equal proportion of all reporter-FRAME-tag strains. The reporter strain mixture was inoculated in fresh medium to an Moo of 0.1, grown for 10 hours until reaching an OD₆₀₀ of 2.7, and then inoculated into 96-well plates containing the appropriate media condition (see FIG. 22) to an Moo of 0.3. Cultures were incubated for 6 hours at 30° C. and 800 RPM or placed statically at the indicated inducing temperature, then placed on ice and analyzed by flow cytometry within 2 hours. For each treatment, the events were partitioned with FRAME-tag gates (see above) and assigned to the corresponding promoter. The MFI for each promoter was calculated as the median value of the mTagBFP2 fluorescence. Each MFI was normalized by the geometric mean of the two internal controls (Vandesompele, J. et al., Genome Biol. 3, RESEARCH0034 (2002)) (promoters from TEF1 and ACT 1):

$${nMFI}_{prom . j , treat . k} = \frac{{MFI}_{prom . j , treat . k}}{\sqrt[2]{{MFI}_{ACT 1 , treat . k} \cdot {MFI}_{TEF 1 , treat . k}}}$$

where MFI_{prom.j.treat.k} is the MFI of the j^{th} promoter in the k^{th} treatment. The expression fold-change for each sample was calculated as follows:

log₂(MFI fold change)=log₂(nMFI_{prom.j.treat.k})−log₂(nMFI_{prom.j.control})

where nMFI_{prom.j.control} is the normalized MFI of the j^{th} promoter in the control treatment of standard synthetic dropout medium (2% glucose) at 30° C.

### Results

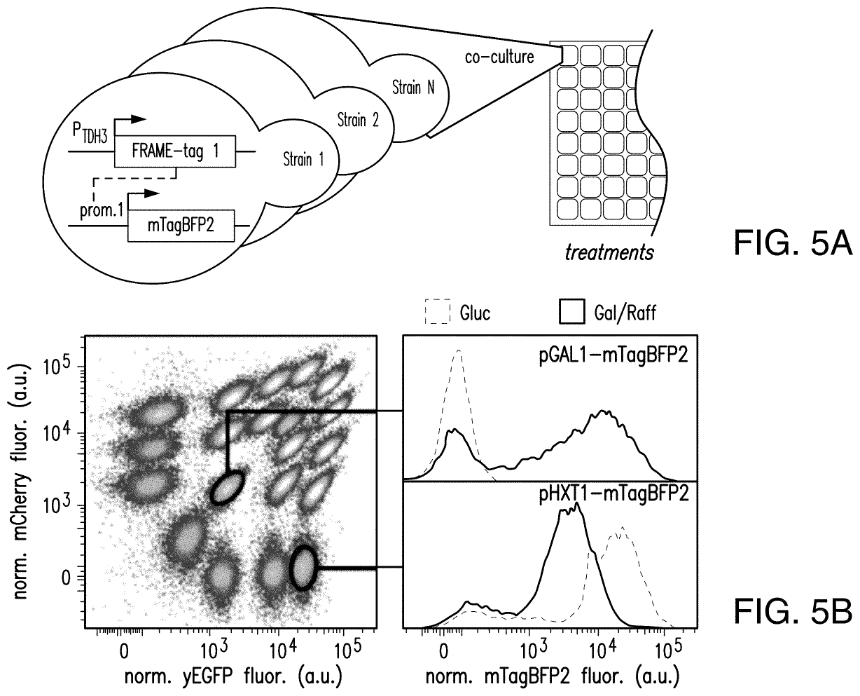

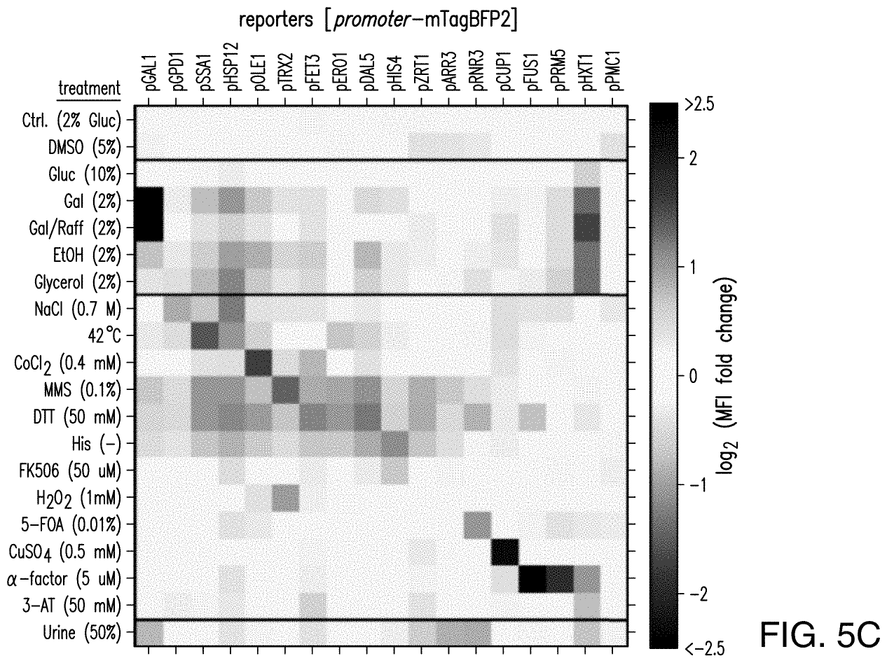

Another feature of FRAME-tags is their compatibility with additional fluorescent reporters. Therefore, strain identification coupled with fluorescent phenotypic reporting within a mixed cell population was explored. To demonstrate this multiplexed reporting capability, a reporter system based on the orthogonal mTagBFP2 was combined with the palette of green-red FRAME-tags. 20 yeast promoters were selected, including 18 environmentally sensitive genes and two housekeeping genes, and cloned these upstream of mTagBFP2 (see strains in Table 3). Each reporter construct was then transformed into one of the 20 green-red FRAME-tagged strains (FIG. 5A). FIG. 5n is a schematic showing green-red FRAME-tags that were used to index the identity of 20 promoters driving the expression of a blue FP for profiling the expression of these promoters in co-culture across 20 treatments.

Transcriptional responses were analyzed from each cell type in multiplex by deconvolution of the sample's bulk blue fluorescence signal using FRAME-tag identities (FIG. 5B). FIG. 5B shows the flow cytometry results for the FRAME-tags used for deconvolution of the bulk blue fluorescence. Histograms were assigned to each individual reporter construct; the mTagBFP2 fluorescent signal was normalized by side scatter and plotted as arbitrary logical units.

This analysis yielded histograms corresponding to the activation profile of individual promoters as well as information on cell type abundance (FIG. 5B and FIG. 22). The expression profiles of these promoters were evaluated in various conditions, including 5 carbon sources, 9 cell stress inducers, two heavy metals, a GPCR agonist and a human biological sample FIG. 22A-U. FIG. 5C shows expression profiles of the 20 yeast promoters from mixed co-cultures following the indicated treatments; heat map represents the log 2 of the fold change of the mean mTagBFP2 fluorescence compared to the control condition (30° C. in SC dropout with 2% glucose); promoter expression for each condition was normalized to pACT1 and pTEF1 control promoters; cultures were analyzed by flow cytometry after 6 hours in the specified treatment. FIG. 22A-U show profile responses of mixtures of promoter-mTagBFP2 reporter strains co-exposed to standard conditions (grey), (A) media with 50 mM dithiothreitol (DTT, red), (B) heat shock at 42° C. (red), (C) media with 400 μM cobalt chloride (CoCl₂, red), (D) 500 μM copper sulfate (CuSO₄, red), (E) an alternate carbon source (2% galactose, red), (F) a mixed carbon source (2% galactose/2% raffinose, red), (G) an increased amount of the carbon source (10% glucose, red), (H) a non-standard carbon source (2% ethanol, red) for 6 hours, (I) a non-standard carbon source (2% glucose, red), (J) standard media containing 5% dimethylslfoxide (DMSO, red), (K) media with 5 μM FK506 (red), (L) media lacking an essential amino acid (-histidine, red), (M) osmotic shock with in media containing 0.7 M sodium chloride (NaCl, red), (N) media with 5 units zymolyase a cell wall-degrading enzyme (red), (O) oxidative shock with in media containing 1 mM hydrogen peroxide (H₂O₂, red), (P) media with 5 μM α-factor a yeast peptide pheromone (red), (Q) media containing 0.1% methyl methanesulfonate a genotoxin (MMS, red), (R) media with 0.01% 5-fluoroorotic acid which is converted to the antimetabolite 5-fluorouracil (5-FOA, red), (S) media containing 50 mM 3-amino-1,2,4-triazole a competitive inhibitor of histidine metabolism (3-AT, red), (T) media with 40 mM theophylline (red), or (U) media containing 50% human urine (red), all for 6 hours. The mTagBFP2 fluorescent signal was normalized by side scatter and plotted as arbitrary logical units.

This confirmed many known yeast transcriptional responses (FIG. 5C). Moreover, because all promoters were measured in each sample, some conditions could be more finely differentiated based on the fingerprint of transcriptional responses (FIG. 5C, compare Gal vs. Gal/Raff). Importantly, FRAME-tag identification was robust in the face of many harsh conditions (e.g., high temperature, osmotic shock, and genotoxic agents), suggesting that FRAME-tags can be used to characterize heterogeneous populations in complex settings.

TABLE 8
DNA sequences of promoters for
multiplexed transcriptional profiling.

| Promoter | Sequence |
|---|---|
| pACT1 | CCTTAAAAACATATGCCTCACCCTAACATATTTTCCAAT |
| | TAACCCTCAATATTTCTCTGTCACCCGGCCTCTATTTTC |
| | CATTTTCTTCTTTACCCGCCACGCGTTTTTTTCTTTCAA |
| | ATTTTTTTTTTCCTTCTTCTTTTTCTTCCACGTCCTCTT |
| | GCATAAATAAATAAACCGTTTTGAAACCAAACTCGCCTC |
| | TCTCTCTCCTTTTTGAAATATTTTTGGGTTTGTTTGATC |
| | CTTTCCTTCCCAATCTCTCTTGTTTAATATATATTCATT |
| | TATATCACGCTCTCTTTTTATCTTCCTTTTTTTCCTCTC |
| | TCTTGTATTTTTCCTTCCCCTTTCTACTCAAACCAAGAA |
| | GAAAAAGAAAAGGTCAATCTTTGTTAAAGAATAGGATCT |
| | TCTACTACATCAGCTTTTAGATTTTTCACGCTTACTGCT |
| | TTTTTCTTCCCAAGATCGAAAATTTACTGtctagaaa |
| pTEF1 | ATAGCTTCAAAATGTTTCTACTCCTTTTTTACTCTTCCA |
| | GATTTTCTCGGACTCCGCGCATCGCCGTACCACTTCAAA |
| | ACACCCAAGCACAGCATACTAAATTTCCCCTCTTTCTTC |
| | CTCTAGGGTGTCGTTAATTACCCGTACTAAAGGTTTGGA |
| | AAAGAAAAAAGAGACCGCCTCGTTTCTTTTTCTTCGTCG |
| | AAAAAGGCAATAAAAATTTTTATCACGTTTCTTTTTCTT |
| | GAAAATTTTTTTTTTTGATTTTTTTCTCTTTCGATGACC |
| | TCCCATTGATATTTAAGTTAATAAACGGTCTTCAATTTC |
| | TCAAGTTTCAGTTTCATTTTTCTTGTTCTATTACAACTT |
| | TTTTTACTTCTTGCTCATTAGAAAGAAAGCATAGCAATC |
| | TAAtctagaaa |
| pGAL1 | ACGGATTAGAAGCCGCCGAGCGGGTGACAGCCCTCCGAA |
| | GGAAGACTCTCCTCCGTGCGTCCTCGTCTTCACCGGTCG |
| | CGTTCCTGAAACGCAGATGTGCCTCGCGCCGCACTGCTC |
| | CGAACAATAAAGATTCTACAATACTAGCTTTTATGGTTA |
| | TGAAGAGGAAAAATTGGCAGTAACCTGGCCCCACAAACC |
| | TTCAAATGAACGAATCAAATTAACAACCATAGGATGATA |
| | ATGCGATTAGTTTTTTAGCCTTATTTCTGGGGTAATTAA |
| | TCAGCGAAGCGATGATTTTTGATCTATTAACAGATATAT |
| | AAATGCAAAAACTGCATAACCACTTTAACTAATACTTTC |
| | AACATTTTCGGTTTGTATTACTTCTTATTCAAATGTAAT |
| | AAAAGTATCAACAAAAAATTGTTAATATACCTCTATACT |
| | TTAACGTCAAGGAGAAAAAACcccggattctagaaa |
| pGPD1 | CTGGGGTTTGAGCAAGTCTAAGTTTACGTAGCATAAAAA |
| | TTCTCGGATTGCGTCAAATAATAAAAAAAGTAACCCCAC |
| | TTCTACTTCTACATCGGAAAAACATTCCATTCACATATC |
| | GTCTTTGGCCTATCTTGTTTTGTCCTCGGTAGATCAGGT |
| | CAGTACAAACGCAACACGAAAGAACAAAAAAAGAAGAAA |
| | ACAGAAGGCCAAGACAGGGTCAATGAGACTGTTGTCCTC |
| | CTACTGTCCCTATGTCTCTGGCCGATCACGCGCCATTGT |
| | CCCTCAGAAACAAATCAAACACCCACACCCCGGGCACCC |
| | AAAGTCCCCACCCACACCACCAATACGTAAACGGGGCGC |
| | CCCCTGCAGGCCCTCCTGCGCGCGGCCTCCCGCCTTGCT |
| | TCTCTCCCCTTCCTTTTCTTTTTCCAGTTTTCCCTATTT |
| | TGTCCCTTTTTCCGCACAACAAGTATCAGAATGGGTTCA |
| | TCAAATCTATCCAACCTAATTCGCACGTAGACTGGCTTG |
| | GTATTGGCAGTTTCGTAGTTATATATATACTACCATGAG |
| | TGAAACTGTTACGTTACCTTAAATTCTTTCTCCCTTTAA |
| | TTTTCTTTTATCTTACTCTCCTACATAAGACATCAAGAA |
| | ACAATTGTATATTGTACACCCCCCCCCTCCACAAACACA |
| | AATATTGATAAtctagaaa |
| pSSA1 | GGCATTTTCGTTCTTGTGGATTGTTGTAAACTTTCCAGA |
| | ACATTCTAGAAAGAAAGCACACGGAACGTTTAGAAGCTG |
| | TCATTTGCGTTTTTTCTCCAGATTTTAGTTGAGAAAGTA |
| | ATTAAATTATTCTTCTTTTTCCAGAACGTTCCATCGGCG |
| | GCAAAAGGGAGAGAAAGAACCCAAAAAGAAGGGGGGCCA |
| | TTTAGATTAGCTGATCGTTTCGAGGACTTCAAGGTTATA |
| | TAAGGGGTGGATTGATGTATCTTCGAGAAGGGATTGAGT |
| | TGTAGTTTCGTTTCCCAATTCTTACTTAAGTTGTTTTAT |
| | TTTCTCTATTTGTAAGATAAGCACATCAAAAGAAAAGTA |
| | ATCAAGTATTACAAGAAACAAAAATtctagaaa |
| pHSP12 | AGTGAAAATCTCCGGGAGCGGGCGGATCCCACTAACGGC |
| | CCAGCCGAAAATGGAAAAAAAGGGTCGGTGATGTGTGGG |
| | TGCCAGCTGGCGGTAGCAATGACGACGTGTTGACGGGCC |
| | CTTGGCTCTTGGGACAAGGACTAGAAGCCAAAAGCCAGA |
| | GGCGGTAAAAATAGCAAGACTAGAATATTGCTGGCATCT |
| | GTTAAGGGGATATGTTGCAACTTGCAGGGGGCGGCACAA |
| | AATAACATAGAAACGTAGTAAAGAGGGGAAAAGGAAAAG |
| | GAAAAGGAAAAGGAAGGAAAAAAACCCATTGACGTAGAA |
| | ATTGAAAGAAGGAAAGGTATACGCAAGCATTAATACAAC |
| | CCACAAACACAGACCAGAAGCACTCTAGACGGAGAGTAA |
| | CTAGATCTACAGCCCCTGGAAAATCGTTTGGTCAACTTT |
| | GAGGTTCCGGTCGTCCCCCTCTTGATCTGAAAGGTCTTT |
| | CTCTAAATCTATATTAAAACGTATAAATAGGACGGTGAA |
| | TTGCGTTCTACTTCCTCAATTGCGTTTGATCTTATTTAA |
| | TCTCTCTCTAATATATAGAAAAAAAAACCATCTGATTAT |
| | TCGATAATCTCAAACAAACAACTCAtctagaaa |
| pOLE1 | CATGTCCCGGGGTTAGCGGGCCCAACAAAGGCGCTTATC |
| | TGGTGGGCTTCCGTAGAAGAAAAAAAGCTGTTGAGCGAG |
| | CTATTTCGGGTATCCCAGCCTTCTCTGCAGACCGCCCCA |
| | GTTGGCTTGGCTCTGGTGCTGTTCGTTAGCATCACATCG |
| | CCTGTGACAGGCAGAGGTAATAACGGCTTAAGGTTCTCT |
| | TCGCATAGTCGGCAGCTTTCTTTCGGACGTTGAACACTC |
| | AACAAACCTTATCTAGTGCCCAACCAGGTGTGCTTCTAC |
| | GAGTCTTGCTCACTCAGACACACCTATCCCTATTGTTAC |
| | GGCTATGGGGATGGCACACAAAGGTGGAAATAATAGTAG |
| | TTAACAATATATGCAGCAAATCATCGGCTCCTGGCTCAT |
| | CGAGTCTTGCAAATCAGCATATACATATATATATGGGGG |
| | CAGATCTTGATTCATTTATTGTTCTATTTCCATCTTTCC |
| | TACTTCTGTTTCCGTTTATATTTTGTATTACGTAGAATA |
| | GAACATCATAGTAATAGATAGTTGTGGTGATCATATTAT |
| | AAACAGCACTAAAACATTACAACAAtctagaaa |
| pTRX2 | ACTTTTACGGGTGGCAACGGAACCAACGTATTTAGAGAT |
| | TGTTTTTTGGTCAAGCGAGGAACCCCTGTTGGCAAAGTT |
| | GCCAGGTATATCATGGGTGGCGAGGTCACCATTGCAAGC |
| | ATTGAAACCGTTGGCGGCGTGAGAGTCAGTGAAGAAAGT |
| | CTTGTTGAGCCCGGTAAGAATGACATACTCGGCTTCAAG |
| | ATCGCTCCAAGATCAGCATAACTTGAGTGCCAGTGAATA |
| | TTAAGTAATCATCAAAGTATATGTGTAATTGTTTATACT |
| | CTTAGTAAAGGATGCTCCCTACAAGGTGGCTCTTTTCTT |
| | ACTAAGCGCGTTCAGTTTCCAGCCAGCCGAAAGAGGGAT |
| | ATCAGTATATAAGAAAGCCATTCGGGGGATGAAAAGCTG |
| | ACAAGAGAATAACGAGGACCAGTTTTTATTTGTTGTCTA |
| | GCAAGAATTATACACGCACACATACACGAGAGTCTACGA |
| | tctagaaa |
| pFET3 | GATAATGCCTTGGCTTGCCTATTTCACGGTTACAGGAAT |
| | AATAACATGTTCATACCGTTTCAGGACCAATAAATGGTC |
| | TTCTGCGAGAGAAAAAGGACACTCTCCGTCCGACAGAAA |
| | TAAGCTTTACTTTCCGGGTGCGAATCAGCCCGTTGCGCC |
| | TGGGGTGGTCCCTACAGTACGCTGAGTCGCCGATAAAGA |
| | CCCTCCGCCTAGAGCTAGGCGAGGCTGACTTAGGCAGGC |
| | CCAACAGGCAAGGCCCATCTTCAAAAGTGCACCCATTTG |
| | CAGGTGCTCTTATTCTCGCCAATTGCGACAGAAAATGAA |
| | GGATGCACTCAAACAGTCGATCCTTCGAGGGAGTATGCC |
| | AAGGCCTCGTGCATGTAGTGCGATTATATATATATATAT |
| | ATATATATATATATGTATGTAAGCAGGCCATGCCCTATA |
| | GCTCTTGTTCTATAAGCGATGGATAGGCATAGGAAACGA |
| | AGAGGACCCCAGTGTAAGGAAGAGTAtctagaaa |
| pERO1 | AAAGAACACGGCGGTAAGAATACGTTCCTTTTTGTGCTG |
| | TGTACACCCGTAAAATTGTACATTATTTATTTCAAAATA |
| | TATAACAGGATCCCTCCAGTGTGTCTGAAATGATGCAAC |
| | TCTGATACTTCAGAGTCGTACCCTTATTAAATACTAATG |
| | CCGAATATAGTCATCCAGTAGCCATAGTTCACACACACA |
| | TTACTTATTCACCACATAAAGAACTAGAAATGCTTGCTA |
| | AATATCGTACCTTGAAGGTAAACATTAAGGCCCCCCAAA |
| | AGCATATATACATACTGGCAACACAAAAAAAGTAAATTG |
| | CCAACCACCTACCTTACATATGGTTTGCCCATCCTACAT |
| | TACCAATACTATCAAGACATTTCTTCTGAAACATATTCA |
| | CAACTGAAACGAGATCATTTTCTTATCTATCTATTGAGT |
| | AATGCTTACTTTTCATATTTTCAATGAACAATAGGATAT |
| | GTAGGAGAATTGATATATTCACTGCGTATCAGAGAAAAG |
| | GTCTACTGACATTTTATGGCAAATGTATTCTACACAAAT |
| | CGAGAATACCACAGACAATGGTACAAGACATACACAAAG |
| | AGAAGACTGTTCTAATTAAACAAATAATATTGAGCTACC |
| | TGCTAAGTATGTCCTTTTCCCTTTGTCCTTTGGTTTCTC |
| | TTATAGAAGACCCTGGAAATTTTTCGCATTTTTCCGGCT |
| | TTGGGCGTTAGTAAGAACAAAAAGAAAAGAAGAGAACAA |
| | AAAAGAAACGATACGGAGTACGTGTCATAAAAACTTGTT |
| | CAATCATCCTTGAAGCTAAGTATAAAGAGCTTGAAAAGG |
| | TTTACCACTTAAACTGGTTATACTATTTCAAGAGTGTAA |
| | ACATTTTATTGCATATACCACAGTAACGTGCAGGTAAAt |
| | ctagaaa |
| pDAL5 | AGCGTTCTCATCAGTCACTTGACAAATGCTCGAGGAGCT |
| | ATCATTTGCTGATAAGGTGCTACAGCGCGCTCCTGCCGC |
| | ACGCTTTGTTCCTTTTCGATAAGAGTCCCTCGCGTTAGT |
| | CTGAGTGAAGTGCGGAATTCAGCAAACGAATAACAATCG |
| | ACCTTATGATCATGTGGATTATCGGGGCAAAAGATTTGG |
| | CCAAGATGTCAGAGAACGTTATCACCAATCACTCACACA |
| | ATTAAGTGGTAGTGTAACTCCGAAGATACGGCTAATACT |
| | TATCATTATCTGGTTTTCCGAATATACAGATTGGATGAA |
| | GTAATATATGTATATAAATGGACCAAGGAAACATCAAAT |
| | TAGGAGATCATGAGGGAAAGGTTTAACATAACAACATTG |
| | AAGAAAACAACAAAACAAGGATtctagaaa |
| pHIS4 | AAACCCATGCACAGTGACTCACGTTTTTTTATCAGTCAT |
| | TCGATATAGAAGGTAAGAAAAGGATATGACTATGAACAG |
| | TAGTATACTGTGTATATAATAGATATGGAACGTTATATT |
| | CACCTCCGATGTGTGTTGTACATACATAAAAATATCATA |
| | GCACAACTGCGCTGTGTAATAGTAATACAATtctagaaa |
| pZRT1 | GGCAAGAGTATTTCAGACTTTCCTAATATGAAAGGACAA |
| | ATTGACACTAATGTCTGATTATGGCCAATTCCTGCGGTA |
| | AATTACACGGCGATTACGGCGACATGAGCTCACATTCAT |
| | CACTCTATGGGACAAATGTTTCCAAACTGGGCGCAACAA |
| | ACACCTGATGTGACTCCTACCCTTTGGACAATGCAGATC |
| | CACGCTACGGCAAATTAGTCAAATGCACTAGAACATGGC |
| | GCAAGTACTTATTGTGACCTTTGGGGTACCGTTACCGTC |
| | AGTTTTCTTCAGCTAAGGCGCGCGCGCCAGATAACTAAA |
| | AAAAAATATAGTTGCTGCTTAAAAAACAATACACCCGTA |
| | CTCTCTTGCCTGTAAAAACCTCGAAGGACCAAAGATACC |
| | CTCAAGGTTCTCATCTGTGCGGTATTCTTCAAATTACAA |
| | TGACATTTCCCAAAATTATCAGATGTGCTCAGGTATCTT |
| | CTCTCCAATGAGATGAGACAGATGAACATATTTGACCTT |
| | GAAGGTCATGGAAAGTAGGTTGAGAGCAAATGTGTAGAA |
| | CGAAATTAAGAAAAAAAGAAATTACGCACGGCATTAGCT |
| | CGATGACTTAGTTATAAATAGAGGCCTGGTATCGGCTGT |
| | CATGATCTCATCTCTTCCCTATTTACAAAAAAACTGCAA |
| | GTATAGACAATAAAACAACAGCACAAAtctagaaa |
| pARR3 | CACGTGCAAAATCTTCTCTTCGAAGATCAACAACTTGAA |
| | AATCCTTCCTCTGATTTTCAATTAGGCCCTTGAGTTGCC |
| | TAGACGTTATGAAACTTACCATTACGCTTGCTGGATTGT |
| | CAAGTTTTCCTCAATATTAGTTTGTTATACATTAGTTTT |
| | TATACCGACTTTTCAAAAGTGTTGATTTAAAATCAAATT |
| | TTGAATGCTCTTAATTATCTTTTTGTTTGATTAATAATC |
| | AACTTTAGCGGCAACGCTCCTTACATAATTATAATGTAA |
| | ACGGAAAAGAATATAAATGAATGCTCTCGTTGTAATTCA |
| | AGAGAACCCAACCAACAAATCATCAGGtctagaaa |
| pRNR3 | GTAATAACAAGCAGGTGGGCGCTTTGAAGAGTATGGTAG |
| | AGGATAAGATCCAGAAGGAAACACTCAAGGGTGTTGTCG |
| | TCGCTGGAGGCGTACTAGCCGGCGCTGTGGCCGTGGCTA |
| | GTTTCTTCTTAAGAAACAAGAGAAGGTAACAAGCACATA |
| | AAAAATCAGCACATACGTACATACATAAGAATGAATCGC |
| | ACGCACGCGTAAACATTTATCATTTAATCTTCAGTTGTT |
| | AGATAAAAAAAAAAAGAAAAGAAAAGAAAGTGAAGGCTT |
| | GTTTCAGTTTGAACTAGGTAGCAGAGCAAGCCCTCGTTC |
| | TTGGCTGCTAATTTTCCTAAAGTAGTAAAAAAAGCCAAG |
| | TTATCTGCCTACGGTTGTCACAGCAACATTGCGTGCCGT |
| | TGTTCTTTTGTTTTTTTTTTTTTTTTTTTTTCGTGGTTG |
| | TCGCAGCAACGACACCTAGGCGCTGCTCAAAGGGGCAAA |
| | AACCCGGTTGCCATGGCGAGGACCAAACGACAAGATGGG |
| | AAAAAAACAATAGTCTATTGTTAAATCGTAATACTGTAT |
| | TGTGAGATGCTGACGCGTTTCGTTTTTCGTGTCAGCGTT |
| | CTTTATATTGTTTCGTGTTCTGCTGCAAAACGTATATAA |
| | ACGCACTGCTATTTTGCCTTCTTTTGCCTTCTTCCTTGC |
| | CTTTCTCTCATCTCATATCCAAGTTGAAATAAATATGAC |
| | AAGCAAGAATAGCAGCAGCAATAAtctagaaa |
| pCUP1 | TCACCACCCTTTATTTCAGGCTGATATCTTAGCCTTGTT |
| | ACTAGTTAGAAAAAGACATTTTTGCTGTCAGTCACTGTC |
| | AAGAGATTCTTTTGCTGGCATTTCTTCTAGAAGCAAAAA |
| | GAGCGATGCGTCTTTTCCGCTGAACCGTTCCAGCAAAAA |
| | AGACTACCAACGCAATATGGATTGTCAGAATCATATAAA |
| | AGAGAAGCAAATAACTCCTTGTCTTGTATCAATTGCATT |
| | ATAATATCTTCTTGTTAGTGCAATATCATATAGAAGTCA |
| | TCGAAATAGATATTAAGAAAAACAAACTGTACAATCAAT |
| | CAATCAATCATCACAtctagaaa |
| pFUS1 | TGCCTCAATCCTTCTTTTGCTTCCATATTTACCATGTGG |
| | ACCCTTTCAAAACAGAGTTGTATCTCTGCAGGATGCCCT |
| | TTTTGACGTATTGAATGGCATAATTGCACTGTCACTTTT |
| | CGCGCTGTCTCATTTTGGTGCGATGATGAAACAAACATG |
| | AAACGTCTGTAATTTGAAACAAATAACGTAATTCTCGGG |
| | ATTGGTTTTATTTAAATGACAATGTAAGAGTGGCTTTGT |
| | AAGGTATGTGTTGCTCTTAAAATATTTGGATACGACATC |
| | CTTTATCTTTTTTCCTTTAAGAGCAGGATATAAGCCATC |
| | AAGtctagaaa |
| pPRM5 | CTCACCCGGATCGTAGTCACATGATCAAATAAATTATTG |
| | CATTACCAATGGCTTCTGTATTAGTTACTGTCCAGGAAA |
| | GGTCTCAATATAACCGGTCACCTTATTTATGATAACAAT |
| | TTTTAACCATTTACCCTTTATTTTTGCAAAGTTATGACC |
| | TTTGGAATGCGGCAGAAAAAAAAAAAATTGATGAAGTAG |
| | TCATCAAACAGGTTTCGGCGAAAGACAGTACAAGAATTG |
| | CCAGCTAAAGCTTTTCTAATATGTTATTCCATCAAATAT |
| | TCACGCTATTAATGCTATCTGATCGATTACTTGGTAAAA |
| | ATAACCAAATGAGTATTGGGTCATATTTTGGAGAAGCGA |
| | AAAGTGCACGGCATACTTAAAGAAGAGAAAAAAATTCTT |
| | AATCAAAGCTTAAGCGGCATCGCATAACTCTGACAAAAG |
| | TATAATCATAGTATGTGGTTTGAGTAAACAACAAGACTG |
| | GCAGGTAAGTTTTTTAAATGAATTTTCTTAGCAGGGTTA |
| | TTGCGGCGCACGTCTATAGGTGATTTACCTTTGTTCCTG |
| | AATATACATGATATTCTTAATGCGACAGCGCCCAGGGAA |
| | GCAAAGAACGCCGACGAAAGAGCGGCGGAGCAAAAGGCG |
| | CAAAGTGACACCTTCCGATCCTGGGAAGTGATCGGTTTC |
| | GCTGTTCGGGCTTTTCTTAGACAAATAAAGTTTAATTTT |
| | CTTGCTTTCTCTTTTTGGCTAAGAATTCTATATTGTTCT |
| | TGAAACAACTTTGTCACACGTTCTAAAAATAAAATGGAA |
| | AATGGAAAATGAAAAATTAGAGAGAAAATGTATTACTGA |
| | AGAATGTGGTAAAGACATTTGAAAAATATTTAATATATT |
| | ACCTGAGATCATTCAAAGGAACGCATCGGTGCAAAGAAA |
| | CAGTTATAGCATTAGAGTTAAGTCAAGAGCACTCATTAT |
| | TACTTTAAAAGGTCGTTGAAAATAGAATAAATTGACACT |
| | CAAAACGCAAGAtctagaaa |
| pHXT1 | TGCAAAAAGCTTCCGATCCTCAAATACAGTGAGAGAAAA |
| | AGCAAACTTGTCTTCATTATTTTTCTGTTTGGCCCTGTC |
| | ACGGTTCTTTTTATGGCCTTCTCGAAGGATATCCGTAGT |
| | CAATTATATTCACGTAGTTGCCAAAAGTAATTTTTGGAA |
| | AACTATTATTCCTCCGAGAAAACCTCACACAGAAATCCT |
| | TGCAGGTCTCATCTGGAATATAATTCCCCCCTCCTGAAG |
| | CAAATTTTTCCTTTGAGCCGGAATTTTTGATATTCCGAG |
| | TTCTTTTTTTCCATTCGCGGAGGTTATTCCATTCCTAAA |
| | CGAGTGGCCACAATGAAACTTCAATTCATATCGACCGAC |
| | TATTTTTCTCCGAACCAAAAAAATAGCAGGGCGAGATTG |
| | GAGCTGCGGAAAAAAGAGGAAAAAATTTTTTCGTAGTTT |
| | TCTTGTGCAAATTAGGGTGTAAGGTTTCTAGGGCTTATT |
| | GGTTCAAGCAGAAGAGACAACAATTGTAGGTCCTAAATT |
| | CAAGGCGGATGTAAGGAGTATTGGTTTCGAAAGTTTTTC |
| | CGAAGCGGCATGGCAGGGACTACTTGCGCATGCGCTCGG |
| | ATTATCTTCATTTTTGCTTGCAAAAACGTAGAATCATGG |
| | TAAATTACATGAAGAATTCTCTTTTTTTTTTTTTTTTTT |
| | TTTTTTTTACCTCTAAAGAGTGTTGACCAACTGAAAAAA |
| | CCCTTCTTCAAGAGAGTTAAACTAAGACTAACCATCATA |
| | ACTTCCAAGGAATTAATCGATATCTTGCACTCCTGATTT |
| | TTCTTCAAAGAGACAGCGCAAAGGATTATGACACTGTTG |
| | CATTGAGTCAAAAGTTTTTCCGAAGTGACCCAGTGCTCT |
| | TTTTTTTTTTCCGTGAAGGACTGACAAATATGCGCACAA |
| | GATCCAATACGTAATGGAAATTCGGAAAAACTAGGAAGA |
| | AATGCTGCAGGGCATTGCCGTGCCGATCTTTTGTCTTTC |
| | AGATATATGAGAAAAAGAATATTCATCAAGTGCTGATAG |
| | AAGAATACCACTCATATGACGTGGGCAGAAGACAGCAAA |
| | CGTAAACATGAGCTGCTGCGACATTTGATGGCTTTTATC |
| | CGACAAGCCAGGAAACTCCACCATTATCTAATGTAGCAA |
| | AATATTTCTTAACACCCGAAGTTGCGTGTCCCCCTCACG |
| | TTTTTAATCATTTGAATTAGTATATTGAAATTATATATA |
| | AAGGCAACAATGTCCCCATAATCAATTCCATCTGGGGTC |
| | TCATGTTCTTTCCCCACCTTAAAATCTATAAAGATATCA |
| | TAATCGTCAACTAGTTGATATACGtctagaaa |
| pPMC1 | GTTTTTACCCGGCAAAGAAGCTTCTCCATCATTTGTCAG |
| | GGGGAAAAGAAAATTAGGAGGCTCAGGCCCTAGAAGCGC |
| | CCTCCACGCTTTTTAAACAAATGCTAATCTTCATAATTC |
| | ATTATCATCGCCACTTGGCATTGCATTTAATGGGCGCCT |
| | TCCAAAAACATTCAAATAGTATCAGCCCCCCCCCAAGGA |
| | AAGGCTTTATTTGAATTGCGTTAAATATTCGATTTCTTT |
| | CTAGCAATTATTACCAAAATAAATTGCAGGCTTAACGAA |
| | AAATTAAATAATCTCTTCAGATTTTTTCACTTCCCCAAA |
| | CTACATTTTAGTCAGGTTGGCACGATTCAATTGAGAAGG |
| | CTTAGGTTAAGAATAAACATTTTTTCATAAATTTGAGGA |
| | AGAAGGGGCTTTTCTGGTAATTTTCATTCAATAGAAAGG |
| | CTTAAGAAAACGTCAAAAAACTCATGCGCCTCCAGTAAA |
| | AACTAAGTGTATTAAGAGAATTGTATCACATATATACGA |
| | CATTGAATTAAAGAAATAAAACTTGAACAAATAAAGTTT |
| | AGAAAAGTGGTTCTAAAAAAAAAAAAACTGTGTGCGTAA |
| | CAAAAAAAATAtctagaaa |

All promoters were cloned to include a short non-native sequence placed immediately upstream of start codon (underlined and lowercase), which includes an XbaI site.

### REFERENCES

1. Strogatz, S. H. Exploring complex networks. Nature 410, 268-276 (2001).
2. Cho, I. & Blaser, M. J. The human microbiome: at the interface of health and disease. Nat. Rev. Genet. 13, 260-270 (2012).
3. Chattopadhyay, P. K., Gierahn, T. M., Roederer, M. & Love, J. C. Single-cell technologies for monitoring immune systems. Nat. Immunol. 15, 128-135 (2014).
4. Spanogiannopoulos, P., Bess, E. N., Carmody, R. N. & Turnbaugh, P. J. The microbial pharmacists within us: a metagenomic view of xenobiotic metabolism. Nat. Rev. Microbiol. 14, 273-287 (2016).
5. Buffie, C. G. et al. Precision microbiome reconstitution restores bile acid mediated resistance to Clostridium difficile. Nature 517, 205-208 (2015).
6. Marchesi, J. R. et al. Towards the Human Colorectal Cancer Microbiome. PLOS ONE 6, e20447 (2011).
7. Fan, H. C., Fu, G. K. & Fodor, S. P. A. Combinatorial labeling of single cells for gene expression cytometry. Science 347, 1258367 (2015).
8. Yu, C. et al. High-throughput identification of genotype-specific cancer vulnerabilities in mixtures of barcoded tumor cell lines. Nat. Biotechnol. 34, 419-423 (2016).
9. Bhang, H. C. et al. Studying clonal dynamics in response to cancer therapy using high-complexity barcoding. Nat. Med. 21, 440-448 (2015).
10. Krutzik, P. O. & Nolan, G. P. Fluorescent cell barcoding in flow cytometry allows high-throughput drug screening and signaling profiling. Nat. Methods 3, 361-368 (2006).
11. Perfetto, S. P., Chattopadhyay, P. K. & Roederer, M. Seventeen-colour flow cytometry: unravelling the immune system. Nat. Rev. Immunol. 4, 648-655 (2004).
12. Han, M., Gao, X., Su, J. Z. & Nie, S. Quantum-dot-tagged microbeads for multiplexed optical coding of biomolecules. Nat. Biotechnol. 19, 631-635 (2001).
13. Levy, S. F. et al. Quantitative evolutionary dynamics using high-resolution lineage tracking. Nature 519, 181-186 (2015).
14. Blundell, J. R. & Levy, S. F. Beyond genome sequencing: Lineage tracking with barcodes to study the dynamics of evolution, infection, and cancer. Genomics 104, 417-430 (2014).
15. La Manno, G. et al. RNA velocity of single cells. Nature 560, 494-498 (2018).
16. Elowitz, M. & Lim, W. A. Build life to understand it. Nature 468, 889-890 (2010).
17. Chen, Y., Kim, J. K., Hirning, A. J., Josić, K. & Bennett, M. R. Emergent genetic oscillations in a synthetic microbial consortium. Science 349, 986-989 (2015).
18. Song, H., Ding, M.-Z., Jia, X.-Q., Ma, Q. & Yuan, Y.-J. Synthetic microbial consortia: from systematic analysis to construction and applications. Chem. Soc. Rev. 43, 6954-6981 (2014).
19. Shou, W., Ram, S. & Vilar, J. M. G. Synthetic cooperation in engineered yeast populations. Proc. Natl. Acad. Sci. 104, 1877-1882 (2007).
20. Kim, H. J., Boedicker, J. Q., Choi, J. W. & Ismagilov, R. F. Defined spatial structure stabilizes a synthetic multispecies bacterial community. Proc. Natl. Acad. Sci. 105, 18188-18193 (2008).
21. Basu, S., Gerchman, Y., Collins, C. H., Arnold, F. H. & Weiss, R. A synthetic multicellular system for programmed pattern formation. Nature 434, 1130-1134 (2005).
22. Rodriguez, E. A. et al. The Growing and Glowing Toolbox of Fluorescent and Photoactive Proteins. Trends Biochem. Sci. 42, 111-129 (2017).
23. Telford, W. G., Hawley, T., Subach, F., Verkhusha, V. & Hawley, R. G. Flow cytometry of fluorescent proteins. Methods 57,318-330 (2012).
24. Livet, J. et al. Transgenic strategies for combinatorial expression of fluorescent proteins in the nervous system. Nature 450, 56-62 (2007).
25. Chen, R. et al. A Barcoding Strategy Enabling Higher-Throughput Library Screening by Microscopy. ACS Synth. Biol. 4, 1205-1216 (2015).
26. Weber, K. et al. RGB marking facilitates multicolor clonal cell tracking. Nat. Med. 17, 504-509 (2011).
27. Brierley, I. Ribosomal frameshifting on viral RNAs. J. Gen. Virol. 76, 1885-1892 (1995).
28. Anzalone, A. V., Lin, A. J., Zairis, S., Rabadan, R. & Cornish, V. W. Reprogramming eukaryotic translation with ligand-responsive synthetic RNA switches. Nat. Methods 13, 453-458 (2016).
29. Maheshri, N. & O'Shea, E. K. Living with Noisy Genes: How Cells Function Reliably with Inherent Variability in Gene Expression. Annu. Rev. Biophys. Biomol. Struct. 36, 413-434 (2007).
30. Elowitz, M. B., Levine, A. J., Siggia, E. D. & Swain, P. S. Stochastic Gene Expression in a Single Cell. Science 297, 1183-1186 (2002).
31. Subach, o. M., Cranfill, P. J., Davidson, M. W. & Verkhusha, V. V. An Enhanced Monomeric Blue Fluorescent Protein with the High Chemical Stability of the Chromophore. PLoS ONE 6, e28674 (2011).
32. Finak, G. et al. OpenCyto: An Open Source Infrastructure for Scalable, Robust, Reproducible, and Automated, End-to-End Flow Cytometry Data Analysis. PLOS Comput Biol 10, e1003806 (2014).
33. Kalhor, R. et al. Developmental barcoding of whole mouse via homing CRISPR. Science eaat9804 (2018). doi:10.1126/science.aat9804
34. Miyawaki, A. et al. Fluorescent indicators for Ca2+based on green fluorescent proteins and calmodulin. Nature 388, 882-887 (1997).
35. Oldach, L. & Zhang, J. Genetically Encoded Fluorescent Biosensors for

Live-Cell Visualization of Protein Phosphorylation. Chem. Biol. 21, 186-197 (2014).
36. Nuber, S. et al. β-Arrestin biosensors reveal a rapid, receptor-dependent activation/deactivation cycle. Nature 531, 661-664 (2016).
37. Atkins, J. F., Loughran, G., Bhatt, P. R., Firth, A. E. & Baranov, P. V. Ribosomal frameshifting and transcriptional slippage: From genetic steganography and cryptography to adventitious use. Nucleic Acids Res. 44, 7007-7078 (2016).
38. Kong, W., Meldgin, D. R., Collins, J. J. & Lu, T. Designing microbial consortia with defined social interactions. Nat. Chem. Biol. 14, 821 (2018).
39. Zhou, K., Qiao, K., Edgar, S. & Stephanopoulos, G. Distributing a metabolic pathway among a microbial consortium enhances production of natural products. Nat. Biotechnol. 33, 377-383 (2015).
40. Ostrov, N. et al. A modular yeast biosensor for low-cost point-of-care pathogen detection. Sci. Adv. 3, e1603221 (2017).
41. Vidal, M., Braun, P., Chen, E., Boeke, J. D. & Harlow, E. Genetic characterization of a mammalian protein-protein interaction domain by using a yeast reverse two-hybrid system. Proc. Natl. Acad. Sci. 93, 10321-10326 (1996).
42. Gietz, R. D. & Schiestl, R. H. High-efficiency yeast transformation using the LiAc/SS carrier DNA/PEG method. Nat. Protoc. 2, 31-34 (2007).
43. Sakaue-Sawano, A. et al. Visualizing Spatiotemporal Dynamics of Multicellular Cell-Cycle Progression. Cell 132, 487-498 (2008).
44. Goedhart, J. et al. Structure-guided evolution of cyan fluorescent proteins towards a quantum yield of 93%. Nat. Commun. 3, ncomms1738 (2012).
45. Nagai, T. et al. A variant of yellow fluorescent protein with fast and efficient maturation for cell-biological applications. Nat. Biotechnol. 20, 87-90 (2002).
46. Grote, A. et al. JCat: a novel tool to adapt codon usage of a target gene to its potential expression host. Nucleic Acids Res. 33, W526-W531 (2005).
47. Zalatan, J. G., Coyle, S. M., Rajan, S., Sidhu, S. S. & Lim, W. A. Conformational Control of the Ste5 Scaffold Protein Insulates Against MAP Kinase Misactivation. Science 337, 1218-1222 (2012).
48. Zuleta, I. A., Aranda-Diaz, A., Li, H. & El-Samad, H. Dynamic characterization of growth and gene expression using high-throughput automated flow cytometry. Nat. Methods 11, 443-448 (2014).
49. Schindelin, J. et al. Fiji: an open-source platform for biological-image analysis. Nat. Methods 9, 676 (2012).
50. Ferreira, T., Miura, K., Chef, B. & Eglinger, J. Scripts: BAR 1.1.6. Zenodo (2015). doi:10.5281/zenodo.28838
51. Vandesompele, J. et al. Accurate normalization of real-time quantitative RT-PCR data by geometric averaging of multiple internal control genes. Genome Biol. 3, RESEARCH0034 (2002).
52. Finak, G. et al. OpenCyto: an open source infrastructure for scalable, robust, reproducible, and automated, end-to-end flow cytometry data analysis. PLoS Comput. Biol. 10, e1003806 (2014).
53. Janssen, S. & Giegerich, R. The RNA shapes studio. Bioinformatics 31, 423-425 (2015).
54. Anzalone, A. V., Lin, A. J., Zairis, S., Rabadan, R. & Cornish, V. W. Reprogramming eukaryotic translation with ligand-responsive synthetic RNA switches. Nat. Methods 13, 453-458 (2016).

Although the present disclosure and its advantages have been described in detail, it should be understood that various changes, substitutions and alternations can be made herein without departing from the spirit and scope of the disclosure as defined by the following claims.

We claim:

1. A nucleic acid construct for labeling cells, comprising:
(i) a first nucleic acid segment encoding a first fluorescent protein;
(ii) a second nucleic acid segment encoding a second fluorescent protein;
(iii) a third nucleic acid segment comprising a slippery site; and
(iv) a fourth nucleic acid segment comprising a frameshift stimulatory sequence.

2. The nucleic acid construct of claim 1 further comprising a fifth nucleic acid segment encoding a stop codon.

3. The nucleic acid construct of claim 1, wherein (a) the third nucleic acid segment is positioned upstream of the second nucleic acid segment encoding the second fluorescent protein; (b) the third nucleic acid segment is positioned upstream of the first nucleic acid segment encoding the first fluorescent protein; (c) the fourth nucleic acid segment is positioned upstream of the second nucleic acid segment encoding the second fluorescent protein; and/or (d) the fourth nucleic acid segment is positioned upstream of the first nucleic acid segment encoding the first fluorescent protein.

4. The nucleic acid construct of claim 2, wherein (a) the fifth nucleic acid segment is positioned upstream of the second nucleic acid segment encoding the second fluorescent protein; and/or (b) the fifth nucleic acid segment is positioned upstream of the first nucleic acid segment encoding the first fluorescent protein, wherein the stop codon is in frame with the second nucleic acid segment encoding the second fluorescent protein.

5. The nucleic acid construct of claim 1, wherein the first fluorescent protein and the second fluorescent protein are different fluorescent proteins.

6. The nucleic acid construct of claim 5, wherein the first fluorescent protein and the second fluorescent protein are dependently selected from the group consisting of GFP, sfGFP, deGFP, eGFP, Venus, mVenus, YFP, Cerulean, Citrine, CFP, eYFP, eCFP, RFP, mRFP, mCherry, mmCherry, mTurquoise2, mKO2, BFP, mTagBFP2 and mutants or variants thereof.

7. The nucleic acid construct of claim 1 further comprising:
(vi) a sixth nucleic acid segment encoding a third fluorescent protein;
(vii) a seventh nucleic acid segment encoding a second slippery site;
(viii) an eighth nucleic acid segment encoding a second frameshift stimulatory sequence; and
(ix) a ninth nucleic acid segment encoding a second stop codon.

8. The nucleic acid construct of claim 7, wherein the first fluorescent protein, the second fluorescent protein and the third fluorescent protein are dependently selected from the group consisting of GFP, sfGFP, deGFP, eGFP, Venus, mVenus, YFP, Cerulean, Citrine, CFP, eYFP, eCFP, RFP, mRFP, mCherry, mmCherry, mTurquoise2, mKO2, BFP, mTagBFP2 and mutants or variants thereof.

9. The nucleic acid construct of claim 6, wherein the first fluorescent protein is a GFP and the second fluorescent protein is an RFP.

10. The nucleic acid construct of claim 8, wherein the first fluorescent protein is a GFP, the second fluorescent protein is an RFP and the third fluorescent protein is a BFP.

11. A genetically-engineered cell comprising one or more nucleic acid constructs of claim 1.

12. A genetically-engineered cell comprising one or more nucleic acid constructs of claim 7.

13. The genetically-engineered cell of claim 11, wherein the cell is a prokaryotic cell or a eukaryotic cell.

14. The genetically-engineered cell of claim 13, wherein the eukaryotic cell is a fungal cell.

15. The genetically-engineered cell of claim 14, wherein the fungal cell is Saccharomyces cerevisiae.

16. The genetically-engineered cell of claim 11, wherein the first fluorescent protein and the second fluorescent protein are expressed in the cell at a ratio of about 1:1,000 to about 1,000:1 or at a ratio of 1:100 to about 100:1.

17. A kit comprising one or more nucleic acid constructs of claim 1.

18. A kit comprising a genetically-engineered cell of claim 11.

19. A method for labeling a cell, comprising introducing one or more nucleic acid constructs of claim 1 into the cell.

20. The method of claim 19 further comprising expressing two or more fluorescent proteins from the one or more nucleic acid constructs and determining the ratio of fluorescent between the two or more fluorescent proteins expressed in the cell.


SEQUENCE LISTING (98 sequences)

SEQ ID NO: 1 - DNA, 57 bp, Artificial Sequence
  Description of Artificial Sequence Synthetic primer
ctcactaaagggaacaaaagctggagctctagtccttaaaaacatatgcctcaccct

SEQ ID NO: 2 - DNA, 60 bp, Artificial Sequence
  Description of Artificial Sequence Synthetic primer
gcatattttctttaatcaattcagacattttctagacagtaaattttcgatcttgggaag

SEQ ID NO: 3 - DNA, 58 bp, Artificial Sequence
  Description of Artificial Sequence Synthetic primer
ctcactaaagggaacaaaagctggagctctagtatagcttcaaaatgtttctactcct

SEQ ID NO: 4 - DNA, 58 bp, Artificial Sequence
  Description of Artificial Sequence Synthetic primer
gcatattttctttaatcaattcagacattttctagattagattgctatgctttctttc

SEQ ID NO: 5 - DNA, 53 bp, Artificial Sequence
  Description of Artificial Sequence Synthetic primer
ctcactaaagggaacaaaagctggagctctagtagtgaaaatctccgggagcg

SEQ ID NO: 6 - DNA, 60 bp, Artificial Sequence
  Description of Artificial Sequence Synthetic primer
gcatattttctttaatcaattcagacattttctagatgagttgtttgtttgagattatcg

SEQ ID NO: 7 - DNA, 53 bp, Artificial Sequence
  Description of Artificial Sequence Synthetic primer
ctcactaaagggaacaaaagctggagctctagtggcattttcgttcttgtgga

SEQ ID NO: 8 - DNA, 60 bp, Artificial Sequence
  Description of Artificial Sequence Synthetic primer
gcatattttctttaatcaattcagacattttctagaatttttgtttcttgtaatacttga

SEQ ID NO: 9 - DNA, 53 bp, Artificial Sequence
  Description of Artificial Sequence Synthetic primer
ctcactaaagggaacaaaagctggagctctagtctggggtttgagcaagtcta

SEQ ID NO: 10 - DNA, 60 bp, Artificial Sequence
  Description of Artificial Sequence Synthetic primer
gcatattttctttaatcaattcagacattttctagattatcaatatttgtgtttgtggag

SEQ ID NO: 11 - DNA, 55 bp, Artificial Sequence
  Description of Artificial Sequence Synthetic primer
ctcactaaagggaacaaaagctggagctctagttgcctcaatccttcttttgctt

SEQ ID NO: 12 - DNA, 59 bp, Artificial Sequence
  Description of Artificial Sequence Synthetic primer
gcatattttctttaatcaattcagacattttctagacttgatggcttatatcctgctct

SEQ ID NO: 13 - DNA, 52 bp, Artificial Sequence
  Description of Artificial Sequence Synthetic primer
ctcactaaagggaacaaaagctggagctctagtacttttacgggtggcaacg

SEQ ID NO: 14 - DNA, 60 bp, Artificial Sequence
  Description of Artificial Sequence Synthetic primer
gcatattttctttaatcaattcagacattttctagatcgtagactctcgtgtatgtgtgc

SEQ ID NO: 15 - DNA, 54 bp, Artificial Sequence
  Description of Artificial Sequence Synthetic primer
ctcactaaagggaacaaaagctggagctctagtgtaataacaagcaggtgggcg

SEQ ID NO: 16 - DNA, 60 bp, Artificial Sequence
  Description of Artificial Sequence Synthetic primer
gcatattttctttaatcaattcagacattttctagattattgctgctgctattcttgctt

SEQ ID NO: 17 - DNA, 54 bp, Artificial Sequence
  Description of Artificial Sequence Synthetic primer
ctcactaaagggaacaaaagctggagctctagttcaccaccctttatttcaggc

SEQ ID NO: 18 - DNA, 60 bp, Artificial Sequence
  Description of Artificial Sequence Synthetic primer
gcatattttctttaatcaattcagacattttctagatgtgatgattgattgattgattgt

SEQ ID NO: 19 - DNA, 57 bp, Artificial Sequence
  Description of Artificial Sequence Synthetic primer
ctcactaaagggaacaaaagctggagctctagtggcaagagtatttcagactttcct

SEQ ID NO: 20 - DNA, 59 bp, Artificial Sequence
  Description of Artificial Sequence Synthetic primer
gcatattttctttaatcaattcagacattttctagatttgtgctgttgttttattgtct

SEQ ID NO: 21 - DNA, 53 bp, Artificial Sequence
  Description of Artificial Sequence Synthetic primer
ctcactaaagggaacaaaagctggagctctagtgataatgccttggcttgcct

SEQ ID NO: 22 - DNA, 59 bp, Artificial Sequence
  Description of Artificial Sequence Synthetic primer
gcatattttctttaatcaattcagacattttctagatactcttccttacactggggtcc

SEQ ID NO: 23 - DNA, 55 bp, Artificial Sequence
  Description of Artificial Sequence Synthetic primer
ctcactaaagggaacaaaagctggagctctagtcacgtgcaaaatcttctcttcg

SEQ ID NO: 24 - DNA, 58 bp, Artificial Sequence
  Description of Artificial Sequence Synthetic primer
gcatattttctttaatcaattcagacattttctagacctgatgatttgttggttgggt

SEQ ID NO: 25 - DNA, 53 bp, Artificial Sequence
  Description of Artificial Sequence Synthetic primer
ctcactaaagggaacaaaagctggagctctagtaaacccatgcacagtgactc

SEQ ID NO: 26 - DNA, 60 bp, Artificial Sequence
  Description of Artificial Sequence Synthetic primer
gcatattttctttaatcaattcagacattttctagaattgtattactattacacagcgca

SEQ ID NO: 27 - DNA, 54 bp, Artificial Sequence
  Description of Artificial Sequence Synthetic primer
ctcactaaagggaacaaaagctggagctctagtagcgttctcatcagtcacttg

SEQ ID NO: 28 - DNA, 60 bp, Artificial Sequence
  Description of Artificial Sequence Synthetic primer
gcatattttctttaatcaattcagacattttctagaatccttgttttgttgttttcttca

SEQ ID NO: 29 - DNA, 54 bp, Artificial Sequence
  Description of Artificial Sequence Synthetic primer
ctcactaaagggaacaaaagctggagctctagtgtttttacccggcaaagaagc

SEQ ID NO: 30 - DNA, 60 bp, Artificial Sequence
  Description of Artificial Sequence Synthetic primer
gcatattttctttaatcaattcagacattttctagatattttttttgttacgcacacagt

SEQ ID NO: 31 - DNA, 51 bp, Artificial Sequence
  Description of Artificial Sequence Synthetic primer
ctcactaaagggaacaaaagctggagctctagtcatgtcccggggttagcg

SEQ ID NO: 32 - DNA, 59 bp, Artificial Sequence
  Description of Artificial Sequence Synthetic primer
gcatattttctttaatcaattcagacattttctagattgttgtaatgttttagtgctgt

SEQ ID NO: 33 - DNA, 53 bp, Artificial Sequence
  Description of Artificial Sequence Synthetic primer
ctcactaaagggaacaaaagctggagctctagtaaagaacacggcggtaagaa

SEQ ID NO: 34 - DNA, 57 bp, Artificial Sequence
  Description of Artificial Sequence Synthetic primer
gcatattttctttaatcaattcagacattttctagatttacctgcacgttactgtgg

SEQ ID NO: 35 - DNA, 53 bp, Artificial Sequence
  Description of Artificial Sequence Synthetic primer
ctcactaaagggaacaaaagctggagctctagttgcaaaaagcttccgatcct

SEQ ID NO: 36 - DNA, 60 bp, Artificial Sequence
  Description of Artificial Sequence Synthetic primer
gcatattttctttaatcaattcagacattttctagacgtatatcaactagttgacgatta

SEQ ID NO: 37 - DNA, 53 bp, Artificial Sequence
  Description of Artificial Sequence Synthetic primer
ctcactaaagggaacaaaagctggagctctagtctcacccggatcgtagtcac

SEQ ID NO: 38 - DNA, 59 bp, Artificial Sequence
  Description of Artificial Sequence Synthetic primer
gcatattttctttaatcaattcagacattttctagatcttgcgttttgagtgtcaattt

SEQ ID NO: 39 - DNA, 680 bp, Artificial Sequence
  Description of Artificial Sequence Synthetic polynucleotide
cagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaa

SEQ ID NO: 40 - DNA, 21 bp, Artificial Sequence
  Description of Artificial Sequence Synthetic oligonucleotide
atgggttcaggtgaacaatca

SEQ ID NO: 41 - DNA, 30 bp, Artificial Sequence
  Description of Artificial Sequence Synthetic oligonucleotide
ggcagcggcgactacaaggacgacgacgac

SEQ ID NO: 42 - DNA, 24 bp, Artificial Sequence
  Description of Artificial Sequence Synthetic oligonucleotide
ggtgcatctggatcaggacaatca

SEQ ID NO: 43 - DNA, 714 bp, Artificial Sequence
  Description of Artificial Sequence Synthetic polynucleotide
atgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaa

SEQ ID NO: 44 - DNA, 819 bp, Artificial Sequence
  Description of Artificial Sequence Synthetic polynucleotide
atggtttcaaaaggtgaagaagataatatggctattattaaagaatttatgagatttaaagttcatatggaaggttcagttaatggtcatgaatttgaaattgaaggtgaaggtgaaggtagaccatatgaaggtactcaaactgctaaattgaaagttactaaaggtggtccattaccatttgcttgggatattttgtcaccacaatttatgtatggttcaaaagcttatgttaaacatccagctgatattccagattatttaaaattgtcatttccagaaggttttaaatgggaaagagttatgaattttgaagatggtggtgttgttactgttactcaagattcatcattacaagatggtgaatttatttataaagttaaattgagaggtactaattttccatcagatggtccagttatgcaaaaaaaaactatgggttgggaagcttcatcagaaagaatgtatccagaagatggtgctttaaaaggtgaaattaaacaaagattgaaattaaaagatggtggtcattatgatgctgaagttaaaactacttataaagctaaaaaaccagttcaattaccaggtgcttataatgttaatattaaattggatattacttcacataatgaagattatactattgttgaacaatatgaaagagctgaaggtagacattcaactggtggtatggatgaattatataaaagcgctggtgcatctggatcaggacaatcaaaagactttaaactagttgacgcgggtctagtcaacaacgcgttaaacccactagaaggcggttctatgggaatgtctgga

SEQ ID NO: 45 - DNA, 699 bp, Artificial Sequence
  Description of Artificial Sequence Synthetic polynucleotide
atgtctgaattgattaaagaaaatatgcatatgaaattgtacatggaaggtactgttgataatcatcatttcaaatgtacttcagaaggtgaaggtaagccatacgaaggtactcaaactatgagaattaaagttgttgaaggtggtccattgccattcgcttttgatattttggctacttcatttttatatggttcaaagacttttattaatcatactcaaggtattccagattttttcaaacaatcattcccagaaggtttcacttgggaaagagttactacttacgaagatggtggtgttttaactgctactcaagatacttctttgcaagatggttgtttgatttacaatgttaaaattagaggtgttaatttcacttcaaatggtccagttatgcaaaaaaagactttgggttgggaagcttttactgaaactttgtacccagctgatggtggtttggaaggtagaaatgatatggctttgaagttggttggtggttctcatttgattgctaatgctaagactacttatagatcaaagaagccagctaagaatttgaagatgccaggtgtttattacgttgattatagattggaaagaattaaagaagctaataatgaaacttacgttgaacaacatgaagttgctgttgctagatactgtgatttgccatcaaaattgggtcataaattgaat

SEQ ID NO: 46 - DNA, 695 bp, Artificial Sequence
  Description of Artificial Sequence Synthetic polynucleotide
taagcaaatagctaaattatatacgaattaatattatgattaagtgtttacgtgagtgcgatatttttattactatcttatacagttgtatatactctataaaatgagttgtctattaattaacgcgatgaatgctttctgggtttacctctccaacaactctagtttacttctcaatacattcaattgtatttgatttgtcaatacttcatcattaatcaattctatagttttgtttttctcgtttatttccaaatttaatgcatcaattttattattcaatttgtcgttgattttggttaatgattttatggtttgatctctggcattgattgtttgtgttagtttttcattattgataattaaattatttaagttagttatcaactcggtgttttcaagtttcaagttttcaatttctttagagtttattagatttgtcaaagtttctgaattgcttgattggtcctgtagaagagtatttgttgttgtggataattgattcaatttttgagacaattgctggaaggcgttgaaatatctagcatcaatctcatggtttttttcccgagagtctcgtagattcaattgttttaatatatcttgggaccactcttgatttgaactcatggaaattaaactgggtgttgtgttgtggtgtaatgattgtaccccctttgcttataattgtgtgg

SEQ ID NO: 47 - DNA, 714 bp, Artificial Sequence
  Description of Artificial Sequence Synthetic polynucleotide
atgtctaaaggtgaagaattattcactggtgttgttccaattttggttgaattagatggtgatgttaatggtcataaattctctgtttctggtgaaggtgaaggtgatgctacttatggtaagttgactttgaagtttatttgtactactggtaaattgccagttccatggccaactttggttactactttgtcttggggtgttcaatgttttgctagatatccagatcatatgaaacaacatgatttctttaaatctgctatgccagaaggttacgttcaagaaagaactattttctttaaagatgatggtaattacaaaactagagctgaagttaaattcgaaggtgatactttggttaatagaattgaattgaagggtattgatttcaaagaagatggtaatattttgggtcataagttggaatacaattacttctctgataatgtttacattactgctgataagcaaaagaatggtattaaggctaatttcaagattagacataatattgaagatggtggtgttcaattagctgatcattatcaacaaaatactccaattggtgatggtccagttttgttgccagataatcattatttgtctactcaatctaaattgtctaaagatccaaatgaaaaaagagatcatatggttttgttggaatttgttactgctgctggtattactttgggtatggatgaattgtacaaa

SEQ ID NO: 48 - DNA, 714 bp, Artificial Sequence
  Description of Artificial Sequence Synthetic polynucleotide
atgtctaaaggtgaagaattattcactggtgttgttccaattttggttgaattagatggtgatgttaatggtcataaattctctgtttctggtgaaggtgaaggtgatgctacttatggtaagttgactttgaagttgatttgtactactggtaaattgccagttccatggccaactttggttactactttgggttacggtttgcaatgttttgctagatatccagatcatatgaaacaacatgatttctttaaatctgctatgccagaaggttacgttcaagaaagaactattttctttaaagatgatggtaattacaaaactagagctgaagttaaattcgaaggtgatactttggttaatagaattgaattgaagggtattgatttcaaagaagatggtaatattttgggtcataagttggaatacaattacaattctcataatgtttacattactgctgataagcaaaagaatggtattaaggctaatttcaagattagacataatattgaagatggtggtgttcaattagctgatcattatcaacaaaatactccaattggtgatggtccagttttgttgccagataatcattatttgtcttaccaatctaaattgtctaaagatccaaatgaaaaaagagatcatatggttttgttggaatttgttactgctgctggtattactttgggtatggatgaattgtacaaa

SEQ ID NO: 49 - DNA, 651 bp, Artificial Sequence
  Description of Artificial Sequence Synthetic polynucleotide
atgtctgttattaagccagaaatgaaaatgagatattatatggatggttctgttaatggtcatgaatttactattgaaggtgaaggtactggtagaccatatgaaggtcatcaagaaatgactttgagagttactatggctgaaggtggtccaatgccattcgcttttgatttggtttctcatgttttctgttacggtcatagagttttcactaagtacccagaagaaattcctgattattttaagcaagcttttccagaaggtttatcttgggaaagatcattggaatttgaagatggtggttctgcttctgtttctgctcatatttctttgagaggtaatactttctatcataagtctaaatttactggtgttaattttccagctgatggtccaattatgcaaaatcaatctgttgattgggaaccatctactgaaaaaattactgcttctgatggtgttttaaaaggtgatgttactatgtatttgaaattggaaggtggtggtaatcataaatgtcaaatgaaaactacttataaagctgctaaagaaattttggaaatgccaggtgatcattatattggacatagattggttagaaagactgaaggtaatattactgaacaagttgaagatgctgttgctcattct

SEQ ID NO: 50 - DNA, 80 bp, Artificial Sequence
  Description of Artificial Sequence Synthetic oligonucleotide
aagactttaaactagttgacgcgcaactaattcaggccgcgttaaacgttctagaaggcggttctatgggaatgtctgga

SEQ ID NO: 51 - DNA, 80 bp, Artificial Sequence
  Description of Artificial Sequence Synthetic oligonucleotide
aagactttaaactagttgacgcgggtctagtcaacaacgcgttaaacccactagaaggcggttctatgggaatgtctgga

SEQ ID NO: 52 - DNA, 80 bp, Artificial Sequence
  Description of Artificial Sequence Synthetic oligonucleotide
aagactttaaactagttgacgcggctctagtcgcagtcgcgttaaacaagctagaaggcggttctatgggaatgtctgga

SEQ ID NO: 53 - DNA, 80 bp, Artificial Sequence
  Description of Artificial Sequence Synthetic oligonucleotide
aagactttaaactagttgacgcgtcgctaacacgtggcgcgttaaaccatctagaaggcggttctatgggaatgtctgga

SEQ ID NO: 54 - DNA, 80 bp, Artificial Sequence
  Description of Artificial Sequence Synthetic oligonucleotide
aagactttaaactagttgacgcgtcactagtagcgggcgcgttaaacaaactagaaggcggttctatgggaatgtctgga

SEQ ID NO: 55 - DNA, 80 bp, Artificial Sequence
  Description of Artificial Sequence Synthetic oligonucleotide
aagactttaaactagttgacgcggatctagcttgtaacgcgttaaaccagctagaaggcggttctatgggaatgtctgga

SEQ ID NO: 56 - DNA, 80 bp, Artificial Sequence
  Description of Artificial Sequence Synthetic oligonucleotide
aagactttaaactagttgacgcgagtctaggggataacgcgttaaacttcctagaaggcggttctatgggaatgtctgga

SEQ ID NO: 57 - DNA, 80 bp, Artificial Sequence
  Description of Artificial Sequence Synthetic oligonucleotide
aagactttaaactagttgacgcgtcgctaccgcccggcgcgttaaacacactagaaggcggttctatgggaatgtctgga

SEQ ID NO: 58 - DNA, 81 bp, Artificial Sequence
  Description of Artificial Sequence Synthetic oligonucleotide
aaagactttaaactagttgacgcgggtctagtcaacaacgcgttaaacccactagaaggcggttctatgggaatgtctgga

SEQ ID NO: 59 - DNA, 441 bp, Artificial Sequence
  Description of Artificial Sequence Synthetic polynucleotide
atgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcg

SEQ ID NO: 60 - DNA, 345 bp, Artificial Sequence
  Description of Artificial Sequence Synthetic polynucleotide
gccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagtaa

SEQ ID NO: 61 - DNA, 128 bp, Artificial Sequence
  Description of Artificial Sequence Synthetic polynucleotide
ggatccggcagtggttctggcgcactacaaggacgacgacacaagactttaaactagttgacgcgggtctagccgttcccgcgttaaacgtactagaaggcggttctatgggaatgtctggaaagctt

SEQ ID NO: 62 - DNA, 140 bp, Artificial Sequence
  Description of Artificial Sequence Synthetic polynucleotide
ggatccggcagtggttctggcgcactacaaggacgacgacacaagactttaaactagttgacgcgggtttaagtacggtcgcgttaaactcgctagaaggcggttctatgggaaatgtctggaaagcttgccaattttaa

SEQ ID NO: 63 - DNA, 128 bp, Artificial Sequence
  Description of Artificial Sequence Synthetic polynucleotide
ggatccggcagtggttctggcgcactacaaggacgacgacacaagactttaaactagttgacgcgagtctaatgtctgccgcgttaaactcgctagaaggcggttctatgggaatgtctggaaagctt

SEQ ID NO: 64 - DNA, 128 bp, Artificial Sequence
  Description of Artificial Sequence Synthetic polynucleotide
ggatccggcagtggttctggcgcactacaaggacgacgacataagactttaaactagttgacgcggttctaggtcaacccgcgttaaacatcctagaaggcggttctatgggaatgtctggaaagctt

SEQ ID NO: 65 - DNA, 128 bp, Artificial Sequence
  Description of Artificial Sequence Synthetic polynucleotide
ggatccggcagtggttctggcgcactacaaggacgacgacacaagactttaaactagttgacgcggatctaggaggtatcgcgttaaacagcctagaaggcggttctatgggaatgtctggaaagctt

SEQ ID NO: 66 - DNA, 128 bp, Artificial Sequence
  Description of Artificial Sequence Synthetic polynucleotide
ggatccggcagtggttctggcgcactacaaggacgacgacacaagactttaaactagttgacgcgtcgctaccagtgggcgcgttaaatgatctagaaggcggttctatgggaatgtctggaaagctt

SEQ ID NO: 67 - DNA, 128 bp, Artificial Sequence
  Description of Artificial Sequence Synthetic polynucleotide
ggatccggcagtggttctggcgcactacaaggacgacgacacaagactttaaactagttgacgcgggactaggctttgccacgttaaacagcctagaaggcggttctatgggaatgtctggaaagctt

SEQ ID NO: 68 - DNA, 128 bp, Artificial Sequence
  Description of Artificial Sequence Synthetic polynucleotide
ggatccggcagtggttctggcgcactacaaggacgacgacacaagactttaaactagttgacgcgggtctagcacctcgcgcgttaaacgtgctagaaggcggttctatgggaatgtctggaaagctt

SEQ ID NO: 69 - DNA, 128 bp, Artificial Sequence
  Description of Artificial Sequence Synthetic polynucleotide
ggatccggcagtggttctggcgcactacaaggacgacgacacaagactttaaactagttgacgcgggactaggcatatgcgcgttaaacaacctagaaggcggttctatgggaatgtctggaaagctt

SEQ ID NO: 70 - DNA, 466 bp, Artificial Sequence
  Description of Artificial Sequence Synthetic polynucleotide
ccttaaaaacatatgcctcaccctaacatattttccaattaaccctcaatatttctctgtcacccggcctctattttccattttcttctttacccgccacgcgtttttttctttcaaatttttttcttccttcttctttttcttccacgtcctcttgcataaataaataaaccgttttgaaaccaaactcgcctctctctctcctttttgaaatatttttgggtttgtttgatcctttccttcccaatctctcttgtttaatatatattcatttatatcacgctctctttttatcttcctttttttcctctctcttgtattcttccttcccctttctactcaaaccaagaagaaaaagaaaaggtcaatctttgttaaagaataggatcttctactacatcagcttttagatttttcacgcttactgcttttttcttcccaagatcgaaaatttactgtctagaaa

SEQ ID NO: 71 - DNA, 401 bp, Artificial Sequence
  Description of Artificial Sequence Synthetic polynucleotide
atagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctagaaa

SEQ ID NO: 72 - DNA, 465 bp, Artificial Sequence
  Description of Artificial Sequence Synthetic polynucleotide
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggattctagaaa

SEQ ID NO: 73 - DNA, 682 bp, Artificial Sequence
  Description of Artificial Sequence Synthetic polynucleotide
ctggggtttgagcaagtctaagtttacgtagcataaaaattctcggattgcgtcaaataataaaaaaagtaaccccacttctacttctacatcggaaaaacattccattcacatatcgtctttggcctatcttgttttgtcctcggtagatcaggtcagtacaaacgcaacacgaaagaacaaaaaaagaagaaaacagaaggccaagacagggtcaatgagactgttgtcctcctactgtccctatgtctctggccgatcacgcgccattgtccctcagaaacaaatcaaacacccacaccccgggcacccaaagtccccacccacaccaccaatacgtaaacggggcgccccctgcaggccctcctgcgcgcggcctcccgccttgcttctctccccttccttttctttttccagttttccctattttgtccctttttccgcacaacaagtatcagaatgggttcatcaaatctatccaacctaattcgcacgtagactggcttggtattggcagtttcgtagttatatatatactaccatgagtgaaactgttacgttaccttaaattctttctccctttaattttcttttatcttactctcctacataagacatcaagaaacaattgtatattgtacaccccccccctccacaaacacaaatattgataatctagaaa

SEQ ID NO: 74 - DNA, 384 bp, Artificial Sequence
  Description of Artificial Sequence Synthetic polynucleotide
ggcattttcgttcttgtggattgttgtaaactttccagaacattctagaaagaaagcacacggaacgtttagaagctgtcatttgcgttttttctccagattttagttgagaaagtaattaaattattcttctttttccagaacgttccatcggcggcaaaagggagagaaagaacccaaaaagaaggggggccatttagattagctgatcgtttcgaggacttcaaggttatataaggggtggattgatgtatcttcgagaagggattgagttgtagtttcgtttcccaattcttacttaagttgttttattttctctatttgtaagataagcacatcaaaagaaaagtaatcaagtattacaagaaacaaaaattctagaaa

SEQ ID NO: 75 - DNA, 618 bp, Artificial Sequence
  Description of Artificial Sequence Synthetic polynucleotide
agtgaaaatctccgggagcgggcggatcccactaacggcccagccgaaaatggaaaaaaagggtcggtgatgtgtgggtgccagctggcggtagcaatgacgacgtgttgacgggcccttggctcttgggacaaggactagaagccaaaagccagaggcggtaaaaatagcaagactagaatattgctggcatctgttaaggggatatgttgcaacttgcagggggcggcacaaaataacatagaaacgtagtaaagaggggaaaaggaaaaggaaaaggaaaaggaaggaaaaaaacccattgacgtagaaattgaaagaaggaaaggtatacgcaagcattaatacaacccacaaacacagaccagaagcactctagacggagagtaactagatctacagcccctggaaaatcgtttggtcaactttgaggttccggtcgtccccctcttgatctgaaaggtctttctctaaatctatattaaaacgtataaataggacggtgaattgcgttctacttcctcaattgcgtttgatcttatttaatctctctctaatatatagaaaaaaaaaccatctgattattcgataatctcaaacaaacaactcatctagaaa

SEQ ID NO: 76 - DNA, 579 bp, Artificial Sequence
  Description of Artificial Sequence Synthetic polynucleotide
catgtcccggggttagcgggcccaacaaaggcgcttatctggtgggcttccgtagaagaaaaaaagctgttgagcgagctatttcgggtatcccagccttctctgcagaccgccccagttggcttggctctggtgctgttcgttagcatcacatcgcctgtgacaggcagaggtaataacggcttaaggttctcttcgcatagtcggcagctttctttcggacgttgaacactcaacaaaccttatctagtgcccaaccaggtgtgcttctacgagtcttgctcactcagacacacctatccctattgttacggctatggggatggcacacaaaggtggaaataatagtagttaacaatatatgcagcaaatcatcggctcctggctcatcgagtcttgcaaatcagcatatacatatatatatgggggcagatcttgattcatttattgttctatttccatctttcctacttctgtttccgtttatattttgtattacgtagaatagaacatcatagtaatagatagttgtggtgatcatattataaacagcactaaaacattacaacaatctagaaa

SEQ ID NO: 77 - DNA, 476 bp, Artificial Sequence
  Description of Artificial Sequence Synthetic polynucleotide
acttttacgggtggcaacggaaccaacgtatttagagattgttttttggtcaagcgaggaacccctgttggcaaagttgccaggtatatcatgggtggcgaggtcaccattgcaagcattgaaaccgttggcggcgtgagagtcagtgaagaaagtcttgttgagcccggtaagaatgacatactcggcttcaagatcgctccaagatcagcataacttgagtgccagtgaatattaagtaatcatcaaagtatatgtgtaattgtttatactcttagtaaaggatgctccctacaaggtggctcttttcttactaagcgcgttcagtttccagccagccgaaagagggatatcagtatataagaaagccattcgggggatgaaaagctgacaagagaataacgaggaccagtttttatttgttgtctagcaagaattatacacgcacacatacacgagagtctacgatctagaaa

SEQ ID NO: 78 - DNA, 502 bp, Artificial Sequence
  Description of Artificial Sequence Synthetic polynucleotide
gataatgccttggcttgcctatttcacggttacaggaataataacatgttcataccgtttcaggaccaataaatggtcttctgcgagagaaaaaggacactctccgtccgacagaaataagctttactttccgggtgcgaatcagcccgttgcgcctggggtggtccctacagtacgctgagtcgccgataaagaccctccgcctagagctaggcgaggctgacttaggcaggcccaacaggcaaggcccatcttcaaaagtgcacccatttgcaggtgctcttattctcgccaattgcgacagaaaatgaaggatgcactcaaacagtcgatccttcgagggagtatgccaaggcctcgtgcatgtagtgcgattatatatatatatatatatatatatatatgtatgtaagcaggccatgccctatagctcttgttctataagcgatggataggcataggaaacgaagaggaccccagtgtaaggaagagtatctagaaa

SEQ ID NO: 79 - DNA, 904 bp, Artificial Sequence
  Description of Artificial Sequence Synthetic polynucleotide
aaagaacacggcggtaagaatacgttcctttttgtgctgtgtacacccgtaaaattgtacattatttatttcaaaatatataacaggatccctccagtgtgtctgaaatgatgcaactctgatacttcagagtcgtacccttattaaatactaatgccgaatatagtcatccagtagccatagttcacacacacattacttattcaccacataaagaactagaaatgcttgctaaatatcgtaccttgaaggtaaacattaaggccccccaaaagcatatatacatactggcaacacaaaaaaagtaaattgccaaccacctaccttacatatggtttgcccatcctacattaccaatactatcaagacatttcttctgaaacatattcacaactgaaacgagatcattttcttatctatctattgagtaatgcttacttttcatattttcaatgaacaataggatatgtaggagaattgatatattcactgcgtatcagagaaaaggtctactgacattttatggcaaatgtattctacacaaatcgagaataccacagacaatggtacaagacatacacaaagagaagactgttctaattaaacaaataatattgagctacctgctaagtatgtccttttccctttgtcctttggtttctcttatagaagaccctggaaatttttcgcatttttccggctttgggcgttagtaagaacaaaaagaaaagaagagaacaaaaaagaaacgatacggagtacgtgtcataaaaacttgttcaatcatccttgaagctaagtataaagagcttgaaaaggtttaccacttaaactggttatactatttcaagagtgtaaacattttattgcatataccacagtaacgtgcaggtaaatctagaaa

SEQ ID NO: 80 - DNA, 420 bp, Artificial Sequence
  Description of Artificial Sequence Synthetic polynucleotide
agcgttctcatcagtcacttgacaaatgctcgaggagctatcatttgctgataaggtgctacagcgcgctcctgccgcacgctttgttccttttcgataagagtccctcgcgttagtctgagtgaagtgcggaattcagcaaacgaataacaatcgaccttatgatcatgtggattatcggggcaaaagatttggccaagatgtcagagaacgttatcaccaatcactcacacaattaagtggtagtgtaactccgaagatacggctaatacttatcattatctggttttccgaatatacagattggatgaagtaatatatgtatataaatggaccaaggaaacatcaaattaggagatcatgagggaaaggtttaacataacaacattgaagaaaacaacaaaacaaggattctagaaa

SEQ ID NO: 81 - DNA, 195 bp, Artificial Sequence
  Description of Artificial Sequence Synthetic polynucleotide
aaacccatgcacagtgactcacgtttttttatcagtcattcgatatagaaggtaagaaaaggatatgactatgaacagtagtatactgtgtatataatagatatggaacgttatattcacctccgatgtgtgttgtacatacataaaaatatcatagcacaactgcgctgtgtaatagtaatacaattctagaaa

SEQ ID NO: 82 - DNA, 698 bp, Artificial Sequence
  Description of Artificial Sequence Synthetic polynucleotide
ggcaagagtatttcagactttcctaatatgaaaggacaaattgacactaatgtctgattatggccaattcctgcggtaaattacacggcgattacggcgacatgagctcacattcatcactctatgggacaaatgtttccaaactgggcgcaacaaacacctgatgtgactcctaccctttggacaatgcagatccacgctacggcaaattagtcaaatgcactagaacatggcgcaagtacttattgtgacctttggggtaccgttaccgtcagttttcttcagctaaggcgcgcgcgccagataactaaaaaaaaatatagttgctgcttaaaaaacaatacacccgtactctcttgcctgtaaaaacctcgaaggaccaaagataccctcaaggttctcatctgtgcggtattcttcaaattacaatgacatttcccaaaattatcagatgtgctcaggtatcttctctccaatgagatgagacagatgaacatatttgaccttgaaggtcatggaaagtaggttgagagcaaatgtgtagaacgaaattaagaaaaaaagaaattacgcacggcattagctcgatgacttagttataaatagaggcctggtatcggctgtcatgatctcatctcttccctatttacaaaaaaactgcaagtatagacaataaaacaacagcacaaatctagaaa

SEQ ID NO: 83 - DNA, 347 bp, Artificial Sequence
  Description of Artificial Sequence Synthetic polynucleotide
cacgtgcaaaatcttctcttcgaagatcaacaacttgaaaatccttcctctgattttcaattaggcccttgagttgcctagacgttatgaaacttaccattacgcttgctggattgtcaagttttcctcaatattagtttgttatacattagtttttataccgacttttcaaaagtgttgatttaaaatcaaattttgaatgctcttaattatctttttgtttgattaataatcaactttagcggcaacgctccttacataattataatgtaaacggaaaagaatataaatgaatgctctcgttgtaattcaagagaacccaaccaacaaatcatcaggtctagaaa

SEQ ID NO: 84 - DNA, 734 bp, Artificial Sequence
  Description of Artificial Sequence Synthetic polynucleotide
gtaataacaagcaggtgggcgctttgaagagtatggtagaggataagatccagaaggaaacactcaagggtgttgtcgtcgctggaggcgtactagccggcgctgtggccgtggctagtttcttcttaagaaacaagagaaggtaacaagcacataaaaaatcagcacatacgtacatacataagaatgaatcgcacgcacgcgtaaacatttatcatttaatcttcagttgttagataaaaaaaaaaagaaaagaaaagaaagtgaaggcttgtttcagtttgaactaggtagcagagcaagccctcgttcttggctgctaattttcctaaagtagtaaaaaaagccaagttatctgcctacggttgtcacagcaacattgcgtgccgttgttcttttgtttttttttttttttttttttcgtggttgtcgcagcaacgacacctaggcgctgctcaaaggggcaaaaacccggttgccatggcgaggaccaaacgacaagatgggaaaaaaacaatagtctattgttaaatcgtaatactgtattgtgagatgctgacgcgtttcgtttttcgtgtcagcgttctttatattgtttcgtgttctgctgcaaaacgtatataaacgcactgctattttgccttcttttgccttcttccttgcctttctctcatctcatatccaagttgaaataaatatgacaagcaagaatagcagcagcaataatctagaaa

SEQ ID NO: 85 - DNA, 335 bp, Artificial Sequence
  Description of Artificial Sequence Synthetic polynucleotide
tcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcacatctagaaa

SEQ ID NO: 86 - DNA, 323 bp, Artificial Sequence
  Description of Artificial Sequence Synthetic polynucleotide
tgcctcaatccttcttttgcttccatatttaccatgtggaccctttcaaaacagagttgtatctctgcaggatgccctttttgacgtattgaatggcataattgcactgtcacttttcgcgctgtctcattttggtgcgatgatgaaacaaacatgaaacgtctgtaatttgaaacaaataacgtaattctcgggattggttttatttaaatgacaatgtaagagtggctttgtaaggtatgtgttgctcttaaaatatttggatacgacatcctttatcttttttcctttaagagcaggatataagccatcaagtctagaaa

SEQ ID NO: 87 - DNA, 995 bp, Artificial Sequence
  Description of Artificial Sequence Synthetic polynucleotide
ctcacccggatcgtagtcacatgatcaaataaattattgcattaccaatggcttctgtattagttactgtccaggaaaggtctcaatataaccggtcaccttatttatgataacaatttttaaccatttaccctttatttttgcaaagttatgacctttggaatgcggcagaaaaaaaaaaaattgatgaagtagtcatcaaacaggtttcggcgaaagacagtacaagaattgccagctaaagcttttctaatatgttattccatcaaatattcacgctattaatgctatctgatcgattacttggtaaaaataaccaaatgagtattgggtcatattttggagaagcgaaaagtgcacggcatacttaaagaagagaaaaaaattcttaatcaaagcttaagcggcatcgcataactctgacaaaagtataatcatagtatgtggtttgagtaaacaacaagactggcaggtaagttttttaaatgaattttcttagcagggttattgcggcgcacgtctataggtgatttacctttgttcctgaatatacatgatattcttaatgcgacagcgcccagggaagcaaagaacgccgacgaaagagcggcggagcaaaaggcgcaaagtgacaccttccgatcctgggaagtgatcggtttcgctgttcgggcttttcttagacaaataaagtttaattttcttgctttctctttttggctaagaattctatattgttcttgaaacaactttgtcacacgttctaaaaataaaatggaaaatggaaaatgaaaaattagagagaaaatgtattactgaagaatgtggtaaagacatttgaaaaatatttaatatattacctgagatcattcaaaggaacgcatcggtgcaaagaaacagttatagcattagagttaagtcaagagcactcattattactttaaaaggtcgttgaaaatagaataaattgacactcaaaacgcaagatctagaaa

SEQ ID NO: 88 - DNA, 1319 bp, Artificial Sequence
  Description of Artificial Sequence Synthetic polynucleotide
tgcaaaaagcttccgatcctcaaatacagtgagagaaaaagcaaacttgtcttcattatttttctgtttggccctgtcacggttctttttatggccttctcgaaggatatccgtagtcaattatattcacgtagttgccaaaagtaatttttggaaaactattattcctccgagaaaacctcacacagaaatccttgcaggtctcatctggaatataattcccccctcctgaagcaaatttttcctttgagccggaatttttgatattccgagttctttttttccattcgcggaggttattccattcctaaacgagtggccacaatgaaacttcaattcatatcgaccgactatttttctccgaaccaaaaaaatagcagggcgagattggagctgcggaaaaaagaggaaaaaattttttcgtagttttcttgtgcaaattagggtgtaaggtttctagggcttattggttcaagcagaagagacaacaattgtaggtcctaaattcaaggcggatgtaaggagtattggtttcgaaagtttttccgaagcggcatggcagggactacttgcgcatgcgctcggattatcttcatttttgcttgcaaaaacgtagaatcatggtaaattacatgaagaattctcttttttttttttttttttttttttttacctctaaagagtgttgaccaactgaaaaaacccttcttcaagagagttaaactaagactaaccatcataacttccaaggaattaatcgatatcttgcactcctgatttttcttcaaagagacagcgcaaaggattatgacactgttgcattgagtcaaaagtttttccgaagtgacccagtgctctttttttttttccgtgaaggactgacaaatatgcgcacaagatccaatacgtaatggaaattcggaaaaactaggaagaaatgctgcagggcattgccgtgccgatcttttgtctttcagatatatgagaaaaagaatattcatcaagtgctgatagaagaataccactcatatgacgtgggcagaagacagcaaacgtaaacatgagctgctgcgacatttgatggcttttatccgacaagccaggaaactccaccattatctaatgtagcaaaatatttcttaacacccgaagttgcgtgtccccctcacgtttttaatcatttgaattagtatattgaaattatatataaaggcaacaatgtccccataatcaattccatctggggtctcatgttctttccccaccttaaaatctataaagatatcataatcgtcaactagttgatatacgtctagaaa

SEQ ID NO: 89 - DNA, 604 bp, Artificial Sequence
  Description of Artificial Sequence Synthetic polynucleotide
gtttttacccggcaaagaagcttctccatcatttgtcagggggaaaagaaaattaggaggctcaggccctagaagcgccctccacgctttttaaacaaatgctaatcttcataattcattatcatcgccacttggcattgcatttaatgggcgccttccaaaaacattcaaatagtatcagcccccccccaaggaaaggctttatttgaattgcgttaaatattcgatttctttctagcaattattaccaaaataaattgcaggcttaacgaaaaattaaataatctcttcagattttttcacttccccaaactacattttagtcaggttggcacgattcaattgagaaggcttaggttaagaataaacattttttcataaatttgaggaagaaggggcttttctggtaattttcattcaatagaaaggcttaagaaaacgtcaaaaaactcatgcgcctccagtaaaaactaagtgtattaagagaattgtatcacatatatacgacattgaattaaagaaataaaacttgaacaaataaagtttagaaaagtggttctaaaaaaaaaaaaactgtgtgcgtaacaaaaaaaatatctagaaa

SEQ ID NO: 90 - RNA, 10 nt, Artificial Sequence
  Description of Artificial Sequence Synthetic oligonucleotide
uuuaaacuag

SEQ ID NO: 91 - RNA, 44 nt, Artificial Sequence
  Description of Artificial Sequence Synthetic oligonucleotide
cgcgcaacuaauucaggccgcguuaaacguucuagaaggcgguu

SEQ ID NO: 92 - RNA, 44 nt, Artificial Sequence
  Description of Artificial Sequence Synthetic oligonucleotide
cgcgggucuagucaacaacgcguuaaacccacuagaaggcgguu

SEQ ID NO: 93 - RNA, 44 nt, Artificial Sequence
  Description of Artificial Sequence Synthetic oligonucleotide
cgcggcucuagucgcagucgcguuaaacaagcuagaaggcgguu

SEQ ID NO: 94 - RNA, 44 nt, Artificial Sequence
  Description of Artificial Sequence Synthetic oligonucleotide
cgcgucgcuaacacguggcgcguuaaaccaucuagaaggcgguu

SEQ ID NO: 95 - RNA, 44 nt, Artificial Sequence
  Description of Artificial Sequence Synthetic oligonucleotide
cgcgucacuaguagcgggcgcguuaaacaaacuagaaggcgguu

SEQ ID NO: 96 - RNA, 44 nt, Artificial Sequence
  Description of Artificial Sequence Synthetic oligonucleotide
cgcggaucuagcuuguaacgcguuaaaccagcuagaaggcgguu

SEQ ID NO: 97 - RNA, 44 nt, Artificial Sequence
  Description of Artificial Sequence Synthetic oligonucleotide
cgcgagucuaggggauaacgcguuaaacuuccuagaaggcgguu

SEQ ID NO: 98 - RNA, 44 nt, Artificial Sequence
  Description of Artificial Sequence Synthetic oligonucleotide
cgcgucgcuaccgcccggcgcguuaaacacacuagaaggcgguu